(19) United States
(12) Patent Application Publication
(10) Pub. No.: US 2015/0202186 A1
(43) Pub. Date: Jul. 23, 2015

(54) CYSTATHIONINE-(gamma)-LYASE (CSE) INHIBITORS FOR TREATING PAIN

(71) Applicant: SOVA PHARMACEUTICALS, INC., La Jolla, CA (US)

(72) Inventors: Sergio G. DURÓN, San Diego, CA (US); Justin CHAPMAN, San Diego, CA (US); Simon G. SYDSERFF, San Diego, CA (US)

(21) Appl. No.: 14/604,541

(22) Filed: Jan. 23, 2015

Related U.S. Application Data
(60) Provisional application No. 61/930,908, filed on Jan. 23, 2014.

(51) Int. Cl. A61K 31/4245; A61K 45/06; A61K 31/18; A61K 31/41; A61K 31/4196

(52) U.S. Cl. CPC A61K 31/4245; A61K 31/41; A61K 31/4196; A61K 31/18; A61K 45/06

(57) ABSTRACT

Described herein are methods for treating chemotherapy-induced peripheral neuropathy, or human immunodeficiency virus-associated sensory neuropathy, or symptoms thereof, comprising administering to an individual in need thereof a therapeutically effective amount of a cystathionine-γ-lyase (CSE) inhibitor. Also described herein are methods for using such CSE inhibitors in combination with other compounds.

### CROSS-REFERENCE

This application claims the benefit of U.S. provisional application Ser. No. 61/930,908, filed Jan. 23, 2014, which is incorporated by reference in its entirety.

### BACKGROUND OF THE INVENTION

Peripheral neuropathy is a condition of the peripheral nervous system in which damage to peripheral nerves can cause severe pain and a range of symptoms in sufferers including numbness, tingling sensations, burning sensations, parasthesia and muscle weakness in various parts of the body. Peripheral neuropathy can result from a variety of diseases, such as cancer or HIV infection, and as a side-effect of drug treatment regimens, for example, chemotherapeutic agents and anti-HIV drugs. Chemotherapy-induced peripheral neuropathy (CIPN) is a common and potential disabling side effect of many cytotoxic drugs. The neurotoxic effects following treatment with chemotherapeutic agents can be severe, significantly affect a patient's quality of life even long after the treatment has ceased, and be a major dose-limiting side-effect of potentially curative cancer chemotherapy treatment regimens. Peripheral neuropathy is also a very common and disabling problem encountered in HIV infection. It develops primarily in relatively advanced patients with low CD4 counts, and may be exacerbated by the neurotoxicity of several of the drugs commonly used to treat HIV.

Hydrogen sulfide (H₂S) is a recognized endogenous gasotransmitter involved in multiple signaling pathways that impact various aspects of physiological and pathological processes including neuropathic pain. Cystathionine-γ-lyase (CSE) is a key enzyme involved in the generation of H₂S and an important target for therapeutic intervention in H₂S-mediated pathologies and disorders.

### SUMMARY OF THE INVENTION

Described herein is a method for treating chemotherapy-induced peripheral neuropathy, or human immunodeficiency virus-associated sensory neuropathy, or symptoms thereof, comprising administering to an individual in need thereof a therapeutically effective amount of a inhibitor of cystathionine-γ-lyase (CSE). Further described herein is a method for treating chemotherapy-induced peripheral neuropathy, or human immunodeficiency virus-associated sensory neuropathy, or symptoms thereof, comprising administering to an individual in need thereof a therapeutically effective amount of a inhibitor of cystathionine-γ-lyase (CSE), wherein the CSE inhibitor is a compound of Formula (I), (II), (III), (IV), (V), (VI), (VII), (VIII), (IX), or (X).

In one aspect is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (I) having the structure:

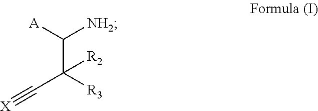

wherein:
A is a carboxylic acid isostere;

### X is CR₁, or N;

R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl; and
R₂ and R₃ are each independently H, substituted or unsubstituted alkyl, or substituted or unsubstituted heteroalkyl; or R₂ and R₃ together with the carbon to which they are attached form a cycloalkyl or heterocycloalkyl ring;
or a pharmaceutically acceptable salt, solvate, or prodrug thereof.

In another aspect is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (II) having the structure:

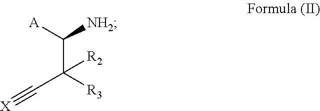

wherein:
A is a carboxylic acid isostere;

### X is CR₁, or N;

R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl; and
R₂ and R₃ are each independently H, substituted or unsubstituted alkyl, or substituted or unsubstituted heteroalkyl; or R₂ and R₃ together with the carbon to which they are attached form a cycloalkyl or heterocycloalkyl ring;
or a pharmaceutically acceptable salt, solvate, or prodrug thereof.

In another aspect is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (III) having the structure:

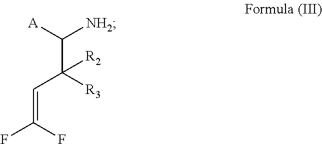

wherein:
A is a carboxylic acid isostere; and
R₂ and R₃ are each independently H, substituted or unsubstituted alkyl, or substituted or unsubstituted heteroalkyl; or R₂ and R₃ together with the carbon to which they are attached form a cycloalkyl or heterocycloalkyl ring;
or a pharmaceutically acceptable salt, solvate, or prodrug thereof.

In another aspect is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (IV) having the structure:

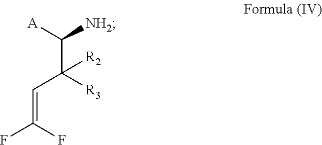

wherein:
A is a carboxylic acid isostere; and
R₂ and R₃ are each independently H, substituted or unsubstituted alkyl, or substituted or unsubstituted heteroalkyl; or R₂ and R₃ together with the carbon to which they are attached form a cycloalkyl or heterocycloalkyl ring;
or a pharmaceutically acceptable salt, solvate, or prodrug thereof.

In another aspect is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (V) having the structure:

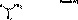

wherein:
A is a carboxylic acid isostere; and
R₁ is substituted or unsubstituted C₃-C₆alkyl, substituted or unsubstituted heteroalkyl, substituted or unsubstituted heterocycloalkyl, substituted or unsubstituted aryl, or substituted or unsubstituted heteroaryl; or a pharmaceutically acceptable salt, solvate, or prodrug thereof.

In another aspect is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VI) having the structure:

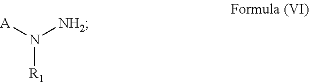

wherein:
R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl; and
A is selected from

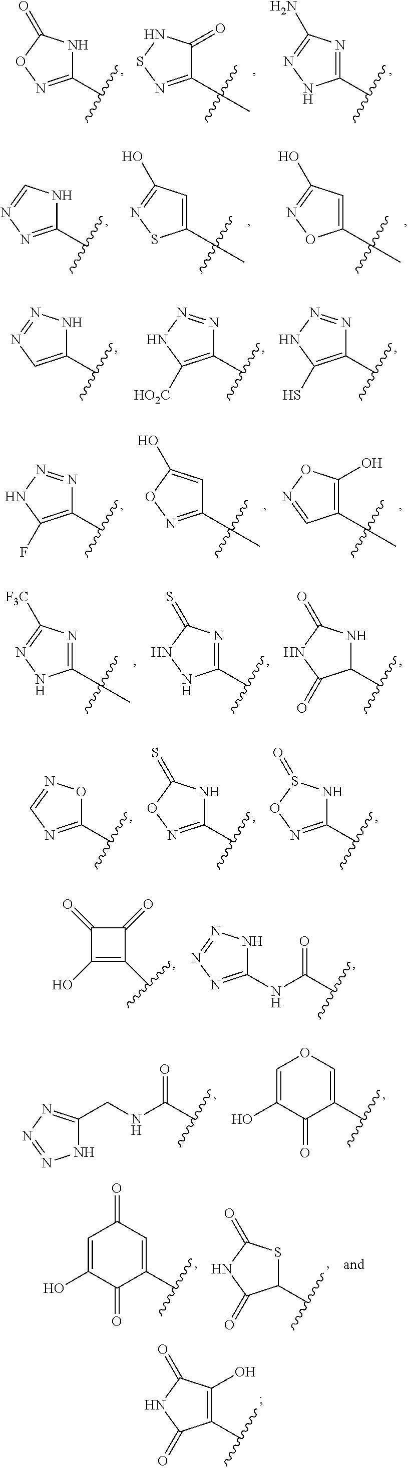

or
a pharmaceutically acceptable salt, solvate, or prodrug thereof.

In another aspect is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VII) having the structure:

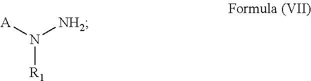

wherein:

R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl; and

A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂; wherein each R₄ is independently H, OH, substituted or unsubstituted alkyl, or substituted or unsubstituted aryl; and R₅ is H or C₁-C₆alkyl; or a pharmaceutically acceptable salt, solvate, or prodrug thereof.

In another aspect is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VIII) having the structure:

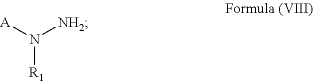

wherein:

A is

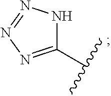 [SMILES: *c1nnn[nH]1]

and
R₁ is substituted or unsubstituted C₂-C₆alkyl, substituted or unsubstituted heteroalkyl, substituted or unsubstituted heterocycloalkyl, substituted or unsubstituted aryl, or substituted or unsubstituted heteroaryl; or a pharmaceutically acceptable salt, solvate, or prodrug thereof.

In another aspect is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (IX) having the structure:

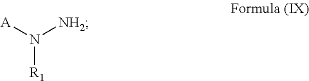

wherein:

A is

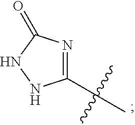 [SMILES: *c1nc(=O)[nH][nH]1]

and
R₁ is H, substituted or unsubstituted C₃-C₆alkyl, substituted or unsubstituted heteroalkyl, substituted or unsubstituted heterocycloalkyl, substituted or unsubstituted aryl, or substituted or unsubstituted heteroaryl; or a pharmaceutically acceptable salt, solvate, or prodrug thereof.

Provided herein, in some embodiments, is a method for treating chemotherapy-induced peripheral neuropathy, or symptoms thereof, comprising administering to an individual in need thereof a therapeutically effective amount of a inhibitor of cystathionine-γ-lyase (CSE), wherein the CSE inhibitor is a compound of Formula (I), (II), (III), (IV), (V), (VI), (VII), (VIII), or (IX). Further described herein is a method for treating chemotherapy-induced peripheral neuropathy, or symptoms thereof, comprising administering to an individual in need thereof a therapeutically effective amount of a inhibitor of cystathionine-γ-lyase (CSE), wherein the CSE inhibitor is a compound of Formula (I), (II), (III), (IV), (V), (VI), (VII), (VIII), or (IX) and the chemotherapy-induced peripheral neuropathy is associated with one or more chemotherapeutic agents selected from docetaxel, paclitaxel, cisplatin, carboplatin, oxaliplatin, vincristine, thalidomide, suramin, bortezomib, rituximab, cyclophosphamide, doxorubicin, abraxane, cabazitaxel, vinorelbine, vinblastine, etoposide, ixabepilone, lenalidomide, pomalidomide, carfilzomib, eribulin, 5-fluorouracil, leucovorin, and gemcitabine. In some embodiments is a method for treating chemotherapy-induced peripheral neuropathy, or symptoms thereof, comprising administering to an individual in need thereof a therapeutically effective amount of a inhibitor of cystathionine-γ-lyase (CSE), wherein the CSE inhibitor is a compound of Formula (I), (II), (III), (IV), (V), (VI), (VII), (VIII), or (IX) and the chemotherapy-induced peripheral neuropathy is associated with one or more chemotherapeutic agents selected from paclitaxel and carboplatin. In some embodiments is a method for treating chemotherapy-induced peripheral neuropathy, or symptoms thereof, comprising administering to an individual in need thereof a therapeutically effective amount of a inhibitor of cystathionine-γ-lyase (CSE), wherein the CSE inhibitor is a compound of Formula (I), (II), (III), (IV), (V), (VI), (VII), (VIII), or (IX) and the chemotherapy-induced peripheral neuropathy is associated with one or two chemotherapeutic agents selected from a taxane and a platinum based therapeutic.

Also provided herein, in some embodiments, is a method for treating human immunodeficiency virus-associated sensory neuropathy, or symptoms thereof, comprising administering to an individual in need thereof a therapeutically effective amount of a inhibitor of cystathionine-γ-lyase (CSE), wherein the CSE inhibitor is a compound of Formula (I), (II), (III), (IV), (V), (VI), (VII), (VIII), (IX), or (X).

Also provided herein, in some embodiments, is a method for treating chemotherapy-induced peripheral neuropathy, or human immunodeficiency virus-associated sensory neuropathy, or symptoms thereof, further comprising administration of a second agent selected from cholinesterase inhibitors, adenosine inhibitors, progestational agents, opioid antagonists, partial opioid agonists, angiotensin receptor blockers, central nervous system stimulants, selective serotonin reuptake inhibitors (SSRIs), dual 5-HT-NE reuptake inhibitors (SNRI's), antidepressants, antihypertensives, calcium channel antagonists, ACE inhibitors, alpha-2 adrenergic agonists, gamma aminobutyric acid agonists, antiepileptic drugs, NSAIDs, steroids, and glutamate antagonists.

In some embodiments, is a method for treating chemotherapy-induced peripheral neuropathy, or human immunodeficiency virus-associated sensory neuropathy, or symptoms thereof, further comprising administrating a second agent selected from acetazolamide, theophylline, progesterone, donepezil, naloxone, nicotine, paroxetine, protriptyline, metoprolol, cilazapril, propranolol, atenolol, hydrochlorothiazide, isradipine, spirapril, doxapram, clonidine, baclofen, sabeluzole, gabapentin, pregablin, duloxetine, morphine, codeine, tramadol, dextropropoxyphene, dihydrocodeine, hydrocodone, oxycodone, losartan, irbesartan, olmesartan, candesartan, telmisartan, azilsartan, naproxen, ketoprofen, acetaminophen, and diclofenac.

### BRIEF DESCRIPTION OF THE DRAWINGS

FIG. 1 shows a graph of tactile allodynia evaluation of Compound A, 30 mpk oral; Compound B, 10, 100 mpk oral; and Morphine, 5 mpk sc; in the chemotherapy-induced peripheral neuropathy with Paclitaxel model (Example 2).

FIG. 2 shows a graph of tactile allodynia evaluation of Compound A, 3, 10, 30 mpk oral; and Morphine, 5 mpk ip; in the chemotherapy-induced peripheral neuropathy with Paclitaxel and Carboplatin model (Example 3).

FIG. 3 shows a graph of tactile allodynia evaluation of Compound B, 10, 30, 100 mpk oral; and Morphine, 5 mpk ip; in the chemotherapy-induced peripheral neuropathy with Paclitaxel and Carboplatin model (Example 3).

### DETAILED DESCRIPTION OF THE INVENTION

A devastating health problem in the United States and abroad is the inadequate treatment of pain. However, little progress has been made in this area of medicine. Neuropathic pain is a particular type of chronic, pathologic pain that has a complex and variable etiology. It is characterized by hyperalgesia (lowered pain threshold and enhanced pain perception) and by allodynia (pain from innocuous mechanical or thermal stimuli). The condition is progressive in nature. Because the hyperesthetic component of neuropathic pain does not respond to the same pharmaceutical interventions as does more generalized and acute forms of pain, development of effective long-term treatment modalities has been problematic. Thus, there is a serious need for new agents and methods of treating neuropathic pain conditions.

Hydrogen sulfide in some embodiments is known to play a role in nociception by sensitizing or directly activating various ion channels (e.g., TRPV channels, TRPA1, NaV and CaV cation channels) potentially contributing to hyperalgesia as well as many other physiological processes including vasodilation (e.g., smooth muscle relaxation and/or opening of vascular smooth muscle K channels), and neuromodulation (e.g., induction of hippocampal long-term potentiation). Endogenous hydrogen sulfide is synthesized through degradation of L-cysteine by cystathionine-gamma-lyase (CSE) or cystathionine-beta synthase (CBS). The enzyme cystathionine-γ-lyase (CSE) converts cystathionine to L-cysteine, yielding pyruvate, ammonia and hydrogen sulfide. Hydrogen sulfide (H₂S) is a gasotransmitter physiologically regulating neuronal transmission and vascular tone. CBS is the predominant H₂S synthesizing enzyme in the brain, while CSE preponderates in the peripheral tissues.

### Chemotherapy-Induced Peripheral Neuropathy (CIPN)

Peripheral neuropathy is a common side-effect of chemotherapy. For example, in patients receiving platinum based chemotherapeutic agents such as carboplatin up to 42% of patients have been reported to have significant peripheral neuropathy and with cisplatin the incidence has been reported to be up to 100%. In the same manner, treatment with taxane based chemotherapeutics such as paclitaxel the incidence of neuropathy has been reported to be up to 83%. The effect is typically dose dependent and co-administration of other neurotoxic chemotherapy agents exacerbates the frequency and severity of neuropathy. This is made more relevant by the current clinical practice of administering platinum doublet or other multidrug combinations. CIPN incidence is also related to cumulative dose or dose-intensities. Patients who already have neuropathic symptoms due to diabetes mellitus, hereditary neuropathies or early treatment with neurotoxic chemotherapy are thought to be more vulnerable for the development of CIPN. Currently, severe or moderate CIPN is alleviated by dose reduction, which may compromise the efficacy of the chemotherapy treatment. Mild or moderate cases still represent an important tolerability problem limiting patient quality of life.

Disclosed herein, in certain embodiments, are methods for treating chemotherapy-induced peripheral neuropathy, or symptoms thereof, in an individual in need thereof. In some embodiments is a method for treating chemotherapy-induced peripheral neuropathy, or symptoms thereof, comprising administering to an individual in need thereof a therapeutically effective amount of an inhibitor of cystathionine-γ-lyase (CSE). In some embodiments is a method for treating chemotherapy-induced peripheral neuropathy, or symptoms thereof, comprising administering to an individual in need thereof a therapeutically effective amount of a inhibitor of cystathionine-γ-lyase (CSE), wherein the CSE inhibitor is a compound of Formula (I), (II), (III), (IV), (V), (VI), (VII), (VIII), (IX), or (X).

Further described herein is a method for treating chemotherapy-induced peripheral neuropathy, or symptoms thereof, comprising administering to an individual in need thereof a therapeutically effective amount of a inhibitor of cystathionine-γ-lyase (CSE), wherein the CSE inhibitor is a compound of Formula (I), (II), (III), (IV), (V), (VI), (VII), (VIII), (IX), or (X) and the chemotherapy-induced peripheral neuropathy is associated with one or more chemotherapeutic agents selected from docetaxel, paclitaxel, cisplatin, carboplatin, oxaliplatin, vincristine, thalidomide, suramin, bortezomib, rituximab, cyclophosphamide, doxorubicin, abraxane, cabazitaxel, vinorelbine, vinblastine, etoposide, ixabepilone, lenalidomide, pomalidomide, carfilzomib, eribulin, 5-fluorouracil, leucovorin, and gemcitabine. In some embodiments the chemotherapy-induced peripheral neuropathy is associated with docetaxel. In some embodiments the chemotherapy-induced peripheral neuropathy is associated with paclitaxel. In some embodiments the chemotherapy-induced peripheral neuropathy is associated with cisplatin. In some embodiments the chemotherapy-induced peripheral neuropathy is associated with carboplatin. In some embodiments the chemotherapy-induced peripheral neuropathy is associated with oxaliplatin. In some embodiments the chemotherapy-induced peripheral neuropathy is associated with vincristine. In some embodiments the chemotherapy-induced peripheral neuropathy is associated with thalidomide. In some embodiments the chemotherapy-induced peripheral neuropathy is associated with suramin. In some embodiments the chemotherapy-induced peripheral neuropathy is associated with bortezomib. In some embodiments the chemotherapy-induced peripheral neuropathy is associated with rituximab. In some embodiments the chemotherapy-induced peripheral neuropathy is associated with cyclophosphamide. In some embodiments the chemotherapy-induced peripheral neuropathy is associated with doxorubicin. In some embodiments the chemotherapy-induced peripheral neuropathy is associated with abraxane. In some embodiments the chemotherapy-induced peripheral neuropathy is associated with cabazitaxel. In some embodiments the chemotherapy-induced peripheral neuropathy is associated with vinorelbine. In some embodiments the chemotherapy-induced peripheral neuropathy is associated with vinblastine. In some embodiments the chemotherapy-induced peripheral neuropathy is associated with etoposide. In some embodiments the chemotherapy-induced peripheral neuropathy is associated with ixabepilone. In some embodiments the chemotherapy-induced peripheral neuropathy is associated with lenalidomide. In some embodiments the chemotherapy-induced peripheral neuropathy is associated with pomalidomide. In some embodiments the chemotherapy-induced peripheral neuropathy is associated with carfilzomib. In some embodiments the chemotherapy-induced peripheral neuropathy is associated with eribulin. In some embodiments the chemotherapy-induced peripheral neuropathy is associated with 5-fluorouracil. In some embodiments the chemotherapy-induced peripheral neuropathy is associated with leucovorin. In some embodiments the chemotherapy-induced peripheral neuropathy is associated with gemcitabine.

In some embodiments is a method for treating chemotherapy-induced peripheral neuropathy, or symptoms thereof, comprising administering to an individual in need thereof a therapeutically effective amount of a inhibitor of cystathionine-γ-lyase (CSE), wherein the CSE inhibitor is a compound of Formula (I), (II), (III), (IV), (V), (VI), (VII), (VIII), (IX), or (X) and the chemotherapy-induced peripheral neuropathy is associated with one or more chemotherapeutic agents selected from paclitaxel and carboplatin. In some embodiments is a method for treating chemotherapy-induced peripheral neuropathy, or symptoms thereof, comprising administering to an individual in need thereof a therapeutically effective amount of a inhibitor of cystathionine-γ-lyase (CSE), wherein the CSE inhibitor is a compound of Formula (I), (II), (III), (IV), (V), (VI), (VII), (VIII), (IX), or (X) and the chemotherapy-induced peripheral neuropathy is associated with one or two chemotherapeutic agents selected from a taxane and a platinum based therapeutic.

Further described herein is a method for treating chemotherapy-induced peripheral neuropathy, or symptoms thereof, comprising administering to an individual in need thereof a therapeutically effective amount of a inhibitor of cystathionine-γ-lyase (CSE), wherein the CSE inhibitor is a compound of Formula (I), (II), (III), (IV), (V), (VI), (VII), (VIII), (IX), or (X) and the chemotherapy-induced peripheral neuropathy is associated with one or more chemotherapeutic agents selected from alkylating agents such as nitrogen mustards (e.g. mechlorethamine (nitrogen mustard), chlorambucil, cyclophosphamide (Cytoxan®), ifosfamide, and melphalan); nitrosoureas (e.g. N-Nitroso-N-methylurea, streptozocin, carmustine (BCNU), lomustine, and semustine); alkyl sulfonates (e.g. busulfan); tetrazines (e.g. dacarbazine (DTIC), mitozolomide and temozolomide (Temodar®)); aziridines (e.g. thiotepa, mytomycin and diaziquone); and platinum drugs (e.g. cisplatin, carboplatin, and oxaliplatin); non-classical alkylating agents such as procarbazine and altretamine (hexamethylmelamine); anti-metabolite agents such as 5-fluorouracil (5-FU), 6-mercaptopurine (6-MP), capecitabine (Xeloda®), cladribine, clofarabine, cytarabine (Ara-C®), decitabine, floxuridine, fludarabine, nelarabine, gemcitabine (Gemzar®), hydroxyurea, methotrexate, pemetrexed (Alimta®), pentostatin, thioguanine, Vidaza; anti-microtubule agents such as vinca alkaloids (e.g. vincristine, vinblastine, vinorelbine, vindesine and vinflunine); taxanes (e.g. paclitaxel (Taxol®), docetaxel (Taxotere®)); podophyllotoxin (e.g. etoposide and teniposide); epothilones (e.g. ixabepilone (Ixempra®)); estramustine (Emcyt®); anti-tumor antibiotics such as anthracyclines (e.g. daunorubicin, doxorubicin (Adriamycin®), epirubicin, idarubicin); actinomycin-D; and bleomycin; topoisomerase I inhibitors such as topotecan and irinotecan (CPT-11); topoisomerase II inhibitors such as etoposide (VP-16), teniposide, mitoxantrone, novobiocin, merbarone and aclarubicin; corticosteroids such as prednisone, methylprednisolone (Solumedrol®), and dexamethasone (Decadron®); L-asparaginase; bortezomib (Velcade®); immunotherapeutic agents such as rituximab (Rituxan®)), alemtuzumab (Campath®), thalidomide, lenalidomide (Revlimid®), BCG, interleukin-2, interferon-alfa and cancer vaccines such as Provenge®; hormone therapeutic agents such as fulvestrant (Faslodex®), tamoxifen, toremifene (Fareston®), anastrozole (Arimidex®), exemestan (Aromasin®), letrozole (Femara®), megestrol acetate (Megace®), estrogens, bicalutamide (Casodex®), flutamide (Eulexin®), nilutamide (Nilandron®), leuprolide (Lupron®) and goserelin (Zoladex®); differentiating agents such as retinoids, tretinoin (ATRA or Atralin®), bexarotene (Targretin®) and arsenic trioxide (Arsenox®); and targeted therapeutic agents such as imatinib (Gleevec®), gefitinib (Iressa®)) and sunitinib (Sutent®). In some embodiments, the chemotherapy is a cocktail therapy. Examples of a cocktail chemotherapy includes, but is not limited to, CHOP/R-CHOP (rituxan, cyclophosphamide, hydroxydoxorubicin, vincristine, and prednisone), EPOCH (etoposide, prednisone, vincristine, cyclophosphamide, hydroxydoxorubicin), Hyper-CVAD (cyclophosphamide, vincristine, hydroxydoxorubicin, dexamethasone), FOLFOX (fluorouracil (5-FU), leucovorin, oxaliplatin), ICE (ifosfamide, carboplatin, etoposide), DHAP (high-dose cytarabine [ara-C], dexamethasone, cisplatin), ESHAP (etoposide, methylprednisolone, cytarabine [ara-C], cisplatin) and CMF (cyclophosphamide, methotrexate, fluorouracil).

In some embodiments, the methods comprise administering a compound of Formula (I), (II), (III), (IV), (V), (VI), (VII), (VIII), (IX), or (X) in combination with a second treatment regimen. In some embodiments, the methods comprise administering a compound of Formula (I), (II), (III), (IV), (V), (VI), (VII), (VIII), (IX), or (X) before, simultaneously with, or after a second treatment regimen. In a further embodiment, the CSE inhibitors of Formula (I), (II), (III), (IV), (V), (VI), (VII), (VIII), (IX), or (X) are administered to an individual in need thereof in combination with chemotherapy and a second treatment regimen wherein the second treatment regimen is selected from radiation therapy, hyperthermia therapy, and surgery. In some embodiments, the second treatment regimen is radiation therapy. In some embodiments, the second treatment regimen is hyperthermia therapy. In some embodiments, the second treatment regimen is surgery.

### Human Immunodeficiency Virus-Associated Sensory Neuropathy

Peripheral neuropathy is a very common and disabling problem encountered in HIV infection. While the condition may be exacerbated by the neurotoxicity of drugs commonly used to treat HIV (drugs including, but not limited to, nucleoside reverse transcriptase inhibitors like zalcitabine, didanosine, lamuvidine, stavudine and fialuridine) it is clear that the viral infection itself results in a typical symmetric, painful, distal sensory neuropathy. This entity almost always presents with variable loss of sensation in the feet and a variety of uncomfortable sensations of swelling, prickling, throbbing or other painful sensations in the feet. This may extend up the legs as it worsens and may eventually start to affect the hands. It occurs in around 20% of AIDS patients, and similar symptoms occur in an even greater number when the drug induced neuropathy is included.

Disclosed herein, in certain embodiments, are methods of treating human immunodeficiency virus-associated sensory neuropathy in an individual in need thereof. In some embodiments, is a method for treating human immunodeficiency virus-associated sensory neuropathy, or symptoms thereof, comprising administering to an individual in need thereof a therapeutically effective amount of a inhibitor of cystathionine-γ-lyase (CSE). Also disclosed herein, in certain embodiments, is a method for treating human immunodeficiency virus-associated sensory neuropathy, or symptoms thereof, comprising administering to an individual in need thereof a therapeutically effective amount of a inhibitor of cystathionine-γ-lyase (CSE), wherein the CSE inhibitor is a compound of Formula (I), (II), (III), (IV), (V), (VI), (VII), (VIII), (IX), or (X).

Further disclosed herein, in certain embodiments, are methods of treating human immunodeficiency virus-associated sensory neuropathy or symptoms thereof, comprising administering to an individual in need thereof a therapeutically effective amount of a inhibitor of cystathionine-γ-lyase (CSE), wherein the CSE inhibitor is a compound of Formula (I), (II), (III), (IV), (V), (VI), (VII), (VIII), (IX), or (X) in combination with one or more agents used to treat an HIV infection. In some embodiments the agents for the treatment of HIV infection include, but are not limited to, multi-class combination drugs such as atripla (efavirenz+tenofovir+emtricitabine); complera (eviplera, rilpivirine+tenofovir+emtricitabine); stribild (elvitegravir+cobicistat+tenofovir+emtricitabine); “572-Trii” (dolutegravir+abacavir+lamivudine or DTG+ABC+3TC); nucleoside/nucleotide reverse transcriptase inhibitors (NRTIs) include combivir (zidovudine+lamivudine, AZT+3TC); emtriva (emtricitabine, FTC); epivir (lamivudine, 3TC); epzicom (Livexa, abacavir+lamivudine, ABC+3TC); retrovir (zidovudine, AZT, ZDV); trizivir (abacavir+zidovudine+lamivudine, ABC+AZT+3TC); truvada (tenofovir DF+emtricitabine, TDF+FTC); videx and videx EC (didanosine, ddl); viread (tenofovir disoproxil fumarate, TDF); zerit (stavudine, d4T); ziagen (abacavir, ABC); amadoxovir (AMDX, DAPD); tenofovir alafenamide fumarate (TAF); non-nucleoside reverse transcriptase inhibitors (NNRTIs) include edurant (rilpivirine, RPV, TMC-278); intelence (etravirine, ETR, TMC-125); rescriptor (delavirdine, DLV); sustiva (Stocrin, efavirenz, EFV); viramune and viramune XR (nevirapine, NVP), lersivirine (UK-453061); immune-based therapies include aralen (chloroquine phosphate), dermaVir, interleukin-7, lexgenleucel-T (VRX-496), plaquenil (hydroxychloroquine), proleukin (aldesleukin, IL-2), SB-782-T and Vacc-4x; protease inhibitors such as aptivus (tipranavir, TPV), crixivan (indinavir, IDV), invirase (saquinavir, SQV), kaletra (Aluvia, lopinavir/ritonavir, LPV/r), lexiva (Telzir, fosamprenavir, FPV), norvir (ritonavir, RTV), prezista (darunavir, DRV), reyataz (atazanavir, ATV) and viracept (nelfinavir, NFV); entry inhibitors (including fusion inhibitors) such as fuzeon (enfuvirtide, ENF, T-20), selzentry (Celsentri, maraviroc, UK-427, 857), cenicriviroc (TBR-652, TAK-652), ibalizumab (TNX-355) and PRO140; integrase inhibitors such as isentress (raltegravir, MK-0518), tivicay (dolutegravir, S/GSK-572) and elvitegravir (GS-9137); pharmacokinetic enhancers such as norvir (ritonavir, RTV), cobicistat (GS-9350) and SPI-452; HIV vaccines such as peptide vaccine, recombinant subunit protein vaccine, live vector vaccine, DNA vaccine, viruls-like particle vaccine (pseudovirion vaccine), vaccine combinations, rgp120 (AIDSVAX) (VAX003 and VAX004), ALVAC HIV (vCP1521)/AIDSVAX B/E (gp120) (RV144), Adenovirus type 5 (Ad5)/gag/pol/nef (HVTN 502/Merck 023), Ad5 gag/pol/nef (HVTB 503) and DNA-Ad5 gag/pol/nef/nev (HVTN505); combination therapy to elicit an immune response such as pegylated interferon alfa, hydroxyurea, mycophenolate mofetil (MPA) and its ester derivative mycophenolate mofetil (MMF); ribavirin, IL-2, IL-12, polymer polyethyleneimine (PEI), or a combination thereof; and HIV-related opportunistic infection treatments such as Co-trimoxazole.

In further embodiments described herein, drugs used commonly to treat infections/complications in AIDS patients are associated with neuropathy (drugs including, but not limited to, isoniazid, vincristine, ethambutol, metronidazole, linezolid, and dapsone) and the risk of neuropathy of these drugs is greater in HIV patients particularly those taking nucleoside reverse transcriptase inhibitors.

Conditions Associated with Diabetic Neuropathy

Diabetic neuropathies are neuropathic disorders that are associated with diabetes mellitus. These conditions are thought to result from diabetic microvascular injury involving small blood vessels that supply nerves (vasa nervorum) in addition to macrovascular conditions that can culminate in diabetic neuropathy. A number of conditions may be associated with diabetic neuropathy including third nerve palsy, diabetic amyotrophy, autonomic neuropathy, and thoracoabdominal neuropathy.

Third nerve palsy, or cranial mononeuropathy III—diabetic type, is a mononeuropathy that affects the third cranial (oculomotor) nerve. Cranial mononeuropathy III is the most common cranial nerve disorder in people with diabetes. It is due to damage to the small blood vessels that feed third cranial nerve which controls eye movement.

Diabetic amyotrophy is a disabling neuropathy that usually occurs in patients with type 2 diabetes mellitus in middle or later age. Concomitant weight loss is frequent. Patients usually have not had diabetes for a long time, and glycemic dysregulation is often not severe. The clinical presentation is characterized by sudden, sharp, and asymmetric pain that usually starts in one hip and thigh and subsequently spreads to the other side within weeks to months.

Diabetic autonomic neuropathy (DAN) is a serious and common complication of diabetes. Despite its relationship to an increased risk of cardiovascular mortality and its association with multiple symptoms and impairments, the significance of DAN has not been fully appreciated. Major clinical manifestations of DAN include resting tachycardia, exercise intolerance, orthostatic hypotension, constipation, gastroparesis, erectile dysfunction, sudomotor dysfunction, impaired neurovascular function, “brittle diabetes,” and hypoglycemic autonomic failure. DAN may affect many organ systems throughout the body (e.g., gastrointestinal [GI], genitourinary, and cardiovascular). GI disturbances (e.g., esophageal enteropathy, gastroparesis, constipation, diarrhea, and fecal incontinence) are common, and any section of the GI tract may be affected.

Diabetic thoracoabdominal neuropathy (TAN) syndrome and its clinical presentations and diagnostic criteria have not been widely recognized. This entity manifests primarily as pain along single or multiple intercostal nerves and may mimic thoracic and/or abdominal visceral pathology (eg, coronary artery disease, gallbladder disease, acute appendicitis). Unless the diagnosis is made early, the patient may be unnecessarily subjected to extensive, expensive, and often invasive procedures. While most patients with TAN have type II diabetes, there is no relationship between the duration of disease and the type of diabetic treatment. In most cases the presenting complaint is burning pain in a dermatomal distribution, usually of gradual onset and often intensified at night.

Disclosed herein, in certain embodiments, are methods of treating a condition associated with diabetes mellitus. In some embodiments, is a method for treating a condition associated with diabetes mellitus, or symptoms thereof, comprising administering to an individual in need thereof a therapeutically effective amount of a inhibitor of cystathionine-γ-lyase (CSE). Also disclosed herein, in certain embodiments, is a method for treating a condition associated with diabetes mellitus, or symptoms thereof, comprising administering to an individual in need thereof a therapeutically effective amount of a inhibitor of cystathionine-γ-lyase (CSE), wherein the CSE inhibitor is a compound of Formula (I), (II), (III), (IV), (V), (VI), (VII), (VIII), (IX), or (X). Further disclosed herein is a method for treating third nerve palsy, diabetic amyotrophy, autonomic neuropathy, or thoracoabdominal neuropathy, or symptoms thereof, comprising administering to an individual in need thereof a therapeutically effective amount of a inhibitor of cystathionine-γ-lyase (CSE), wherein the CSE inhibitor is a compound of Formula (I), (II), (III), (IV), (V), (VI), (VII), (VIII), (IX), or (X). In some embodiments, is a method for treating third nerve palsy, or symptoms thereof, comprising administering to an individual in need thereof a therapeutically effective amount of a inhibitor of cystathionine-γ-lyase (CSE), wherein the CSE inhibitor is a compound of Formula (I), (II), (III), (IV), (V), (VI), (VII), (VIII), (IX), or (X). In some embodiments, is a method for treating diabetic amyotrophy, or symptoms thereof, comprising administering to an individual in need thereof a therapeutically effective amount of a inhibitor of cystathionine-γ-lyase (CSE), wherein the CSE inhibitor is a compound of Formula (I), (II), (III), (IV), (V), (VI), (VII), (VIII), (IX), or (X). In some embodiments, is a method for treating autonomic neuropathy, or symptoms thereof, comprising administering to an individual in need thereof a therapeutically effective amount of a inhibitor of cystathionine-γ-lyase (CSE), wherein the CSE inhibitor is a compound of Formula (I), (II), (III), (IV), (V), (VI), (VII), (VIII), (IX), or (X). In some embodiments, is a method for treating thoracoabdominal neuropathy, or symptoms thereof, comprising administering to an individual in need thereof a therapeutically effective amount of a inhibitor of cystathionine-γ-lyase (CSE), wherein the CSE inhibitor is a compound of Formula (I), (II), (III), (IV), (V), (VI), (VII), (VIII), (IX), or (X). In some embodiments, is a method for treating diabetic peripheral neuropathy, or symptoms thereof, comprising administering to an individual in need thereof a therapeutically effective amount of a inhibitor of cystathionine-γ-lyase (CSE), wherein the CSE inhibitor is a compound of Formula (I), (II), (III), (IV), (V), (VI), (VII), (VIII), (IX), or (X). In some embodiments, is a method for treating the pain associated with diabetic peripheral neuropathy comprising administering to an individual in need thereof a therapeutically effective amount of a inhibitor of cystathionine-γ-lyase (CSE), wherein the CSE inhibitor is a compound of Formula (I), (II), (III), (IV), (V), (VI), (VII), (VIII), (IX), or (X).

Peripheral Neuropathy Associated with Lyme Disease and Zoster Infection

Disclosed herein, in certain embodiments, are methods of treating lyme disease in an individual in need thereof. In some embodiments, is a method for treating lyme disease, comprising administering to an individual in need thereof a therapeutically effective amount of a inhibitor of cystathionine-γ-lyase (CSE). Also disclosed herein, in certain embodiments, is a method for treating lyme disease comprising administering to an individual in need thereof a therapeutically effective amount of a inhibitor of cystathionine-γ-lyase (CSE), wherein the CSE inhibitor is a compound of Formula (I), (II), (III), (IV), (V), (VI), (VII), (VIII), (IX), or (X).

Disclosed herein, in certain embodiments, are methods of treating zoster infection in an individual in need thereof. In some embodiments, is a method for treating zoster infection, comprising administering to an individual in need thereof a therapeutically effective amount of a inhibitor of cystathionine-γ-lyase (CSE). Also disclosed herein, in certain embodiments, is a method for treating zoster infection comprising administering to an individual in need thereof a therapeutically effective amount of a inhibitor of cystathionine-γ-lyase (CSE), wherein the CSE inhibitor is a compound of Formula (I), (II), (III), (IV), (V), (VI), (VII), (VIII), (IX), or (X).

### DEFINITIONS

As used herein, the terms “treat”, “treating” or “treatment” include alleviating, abating or ameliorating at least one symptom of a disease or condition, preventing additional symptoms, preventing progression of the condition, inhibiting the disease or condition, e.g., arresting the development of the disease or condition, relieving the disease or condition, causing regression of the disease or condition, relieving a condition caused by the disease or condition, or stopping the symptoms of the disease or condition. In one embodiment, treatment is prophylactic treatment. In another embodiment, treatment refers to therapeutic treatment.

As used herein, “administer” means to provide a treatment, for example to prescribe a treatment, apply a treatment, or distribute a treatment. In some instances, to administer means a medical professional prescribes a treatment which a patient applies (e.g., consumes a medication, or injects a medication). Administration of a medical treatment does not require the immediate or constant supervision of a medical professional.

“Co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one embodiment, the mammal is a human.

A “tissue” comprises two or more cells. The two or more cells may have a similar function and/or function. The tissue may be a connective tissue, epithelial tissue, muscular tissue, or nervous tissue. Alternatively, the tissue is a bone, tendon (both referred to as musculoskeletal grafts), cornea, skin, heart valve, or vein.

An “organ” comprises two or more tissues. The two or more tissues may perform a specific function or group of functions. In some instances, the organ is a lung, mouth, nose, parathyroid gland, pineal gland, pituitary gland, carotid body, salivary gland, skin, gall bladder, pancreas, small intestine, stomach, spleen, spinal cord, thymus, thyroid gland, trachea, uterus, or vermiform appendix. Alternatively, the organ is an adrenal gland, appendix, brain, bladder, kidney, intestine, large intestine, small intestine, liver, heart, or muscle.

The term “CSE inhibitor” encompasses a full or partial inhibitor of CSE enzymatic activity in the synthesis of hydrogen sulfide.

The term “optionally substituted” or “substituted” means that the referenced group substituted with one or more additional group(s). In certain embodiments, the one or more additional group(s) are individually and independently selected from amide, ester, alkyl, cycloalkyl, heteroalkyl, aryl, heteroaryl, heteroalicyclic, hydroxy, alkoxy, aryloxy, alkylthio, arylthio, alkylsulfoxide, arylsulfoxide, ester, alkylsulfone, arylsulfone, cyano, halogen, alkoyl, alkoyloxo, isocyanato, thiocyanato, isothiocyanato, nitro, haloalkyl, haloalkoxy, fluoroalkyl, amino, alkyl-amino, dialkyl-amino, amido. In one embodiment, the referenced group is substituted with one or more halogen. In another embodiment, the referenced group is substituted with one or more alkyl.

An “alkyl” group refers to an aliphatic hydrocarbon group. Reference to an alkyl group includes “saturated alkyl” and/or “unsaturated alkyl”. The alkyl group, whether saturated or unsaturated, includes branched, straight chain, or cyclic groups. By way of example only, alkyl includes methyl, ethyl, propyl, iso-propyl, n-butyl, iso-butyl, sec-butyl, t-butyl, pentyl, iso-pentyl, neo-pentyl, and hexyl. In some embodiments, alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. A “lower alkyl” is a C₁-C₆ alkyl. A “heteroalkyl” group substitutes any one of the carbons of the alkyl group with a heteroatom having the appropriate number of hydrogen atoms attached (e.g., a CH₂ group to an NH group or an O group).

An “alkoxy” group refers to a (alkyl)O— group, where alkyl is as defined herein.

The term “alkylamine” refers to the —N(alkyl)ₓH_{y} group, wherein alkyl is as defined herein and x and y are selected from the group x=1, y=1 and x=2, y=0. When x=2, the alkyl groups, taken together with the nitrogen to which they are attached, optionally form a cyclic ring system.

An “amide” is a chemical moiety with formula C(O)NHR or NHC(O)R, where R is selected from alkyl, cycloalkyl, aryl, heteroaryl (bonded through a ring carbon) and heteroalicyclic (bonded through a ring carbon).

The term “ester” refers to a chemical moiety with formula —C(═O)OR, where R is selected from the group consisting of alkyl, cycloalkyl, aryl, heteroaryl and heteroalicyclic.

As used herein, the term “aryl” refers to an aromatic ring wherein each of the atoms forming the ring is a carbon atom. Aryl rings described herein include rings having five, six, seven, eight, nine, or more than nine carbon atoms. Aryl groups are optionally substituted. Examples of aryl groups include, but are not limited to phenyl, and naphthalenyl.

The term “cycloalkyl” refers to a monocyclic or polycyclic non-aromatic radical, wherein each of the atoms forming the ring (i.e. skeletal atoms) is a carbon atom. In various embodiments, cycloalkyls are saturated, or partially unsaturated. In some embodiments, cycloalkyls are fused with an aromatic ring. Cycloalkyl groups include groups having from 3 to 10 ring atoms. Illustrative examples of cycloalkyl groups include, but are not limited to, the following moieties:

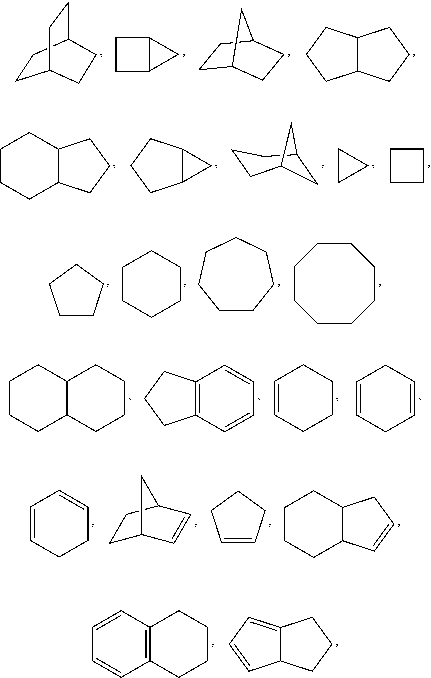

and the like. Monocyclic cycloalkyls include, but are not limited to, cyclopropyl, cyclobutyl, cyclopentyl, cyclohexyl, cycloheptyl, and cyclooctyl. Dicylclic cycloalkyls include, but are not limited to tetrahydronaphthyl, indanyl, tetrahydropentalene or the like. Polycyclic cycloalkyls include adamantane, norbornane or the like. The term cycloalkyl includes “unsaturated nonaromatic carbocyclyl” or “nonaromatic unsaturated carbocyclyl” groups both of which refer to a nonaromatic carbocycle, as defined herein, that contains at least one carbon carbon double bond or one carbon carbon triple bond.

The term “heterocyclic” refers to heteroaromatic and heteroalicyclic groups containing one to four ring heteroatoms each selected from O, S and N. In certain instances, each heterocyclic group has from 4 to 10 atoms in its ring system, and with the proviso that the ring of said group does not contain two adjacent O or S atoms. Non-aromatic heterocyclic groups include groups having 3 atoms in their ring system, but aromatic heterocyclic groups must have at least 5 atoms in their ring system. The heterocyclic groups include benzo-fused ring systems. An example of a 3-membered heterocyclic group is aziridinyl (derived from aziridine). An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. In certain embodiments, heteroaryl groups are monocyclic or polycyclic. Examples of monocyclic heteroaryl groups include and are not limited to:

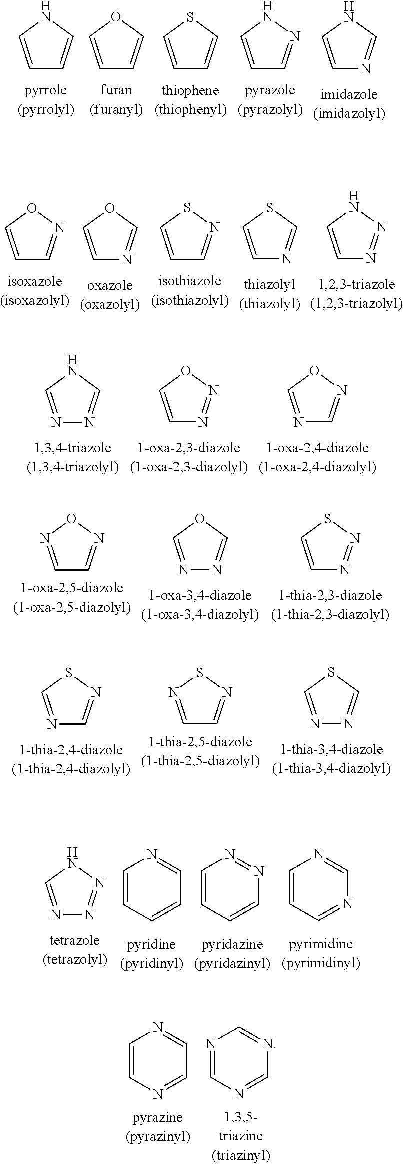

Examples of bicyclic heteroaryl groups include and are not limited to:

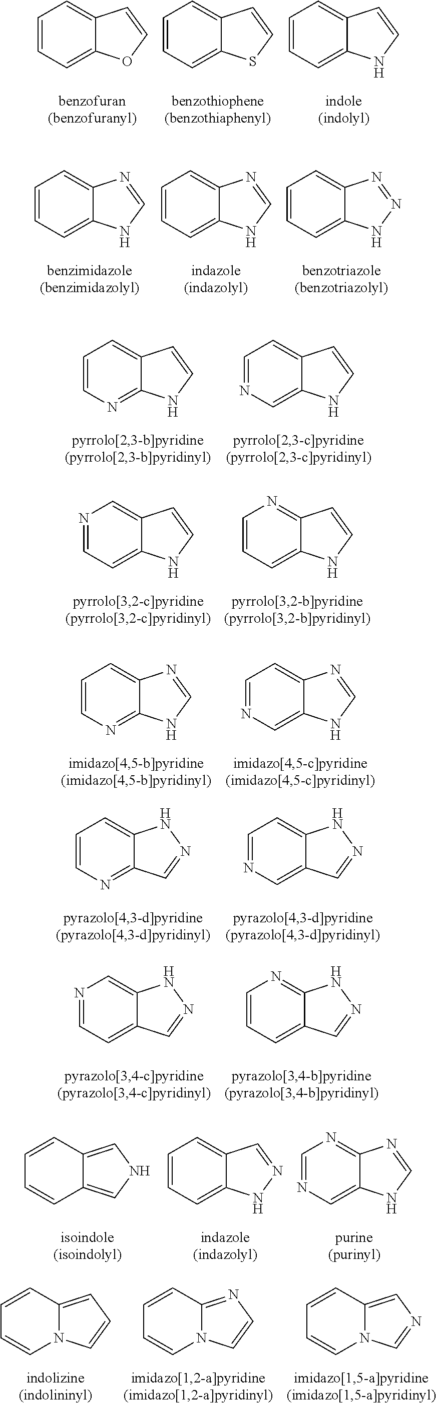

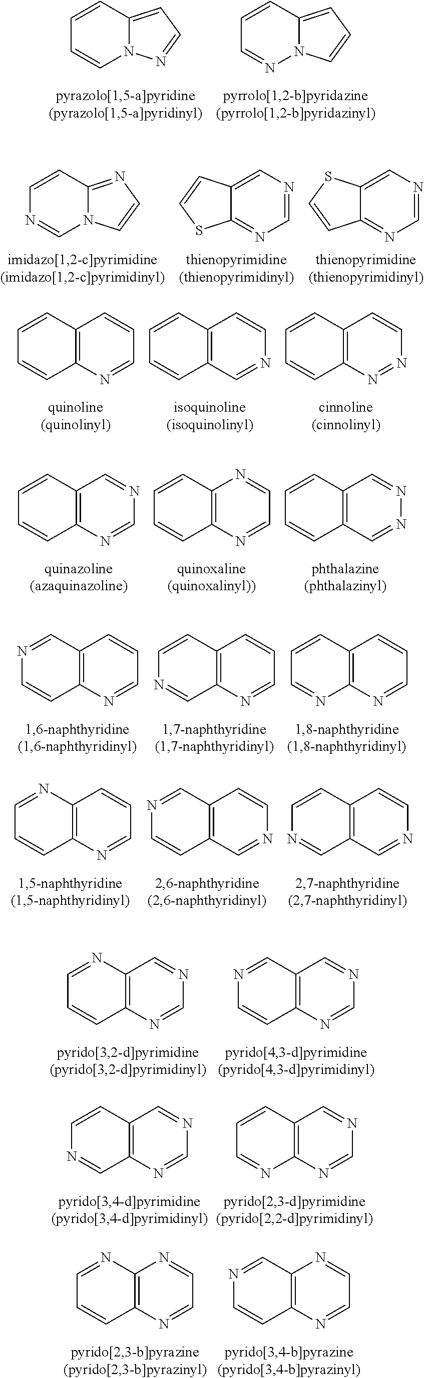

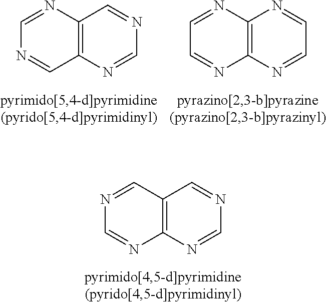 [SMILES: c1ncc2ncncc2n1 ; c1cnc2nccnc2n1 ; c1ncc2cncnc2n1]

or the like.

A “heteroalicyclic” group or “heterocyclo” group or “heterocycloalkyl” group or “heterocyclyl” group refers to a cycloalkyl group, wherein at least one skeletal ring atom is a heteroatom selected from nitrogen, oxygen and sulfur. In various embodiments, heterocycloalkyls are saturated, or partially unsaturated. In some embodiments, the radicals are fused with an aryl or heteroaryl. Example of saturated heterocyloalkyl groups include

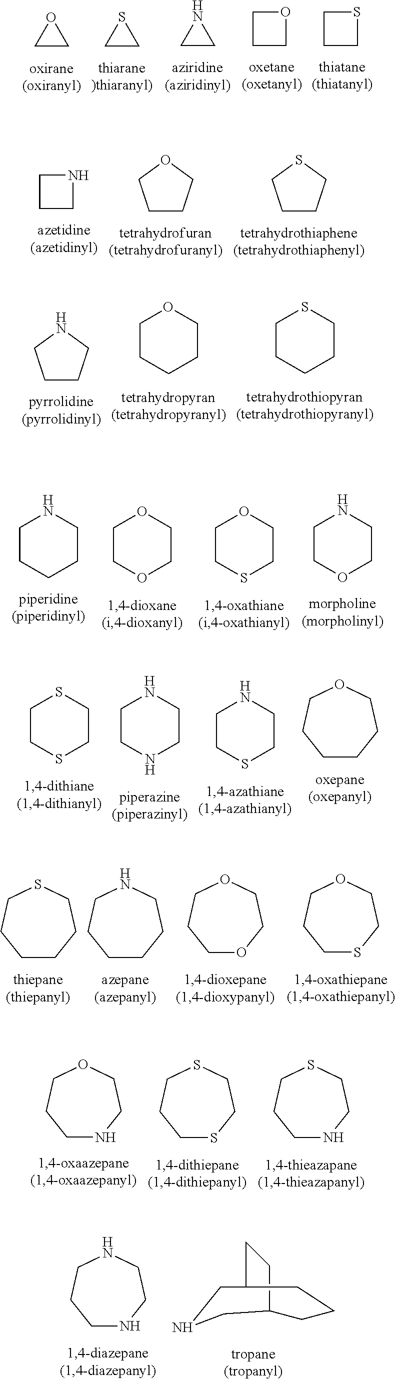

Examples of partially unsaturated heterocyclyl or heterocycloalkyl groups include

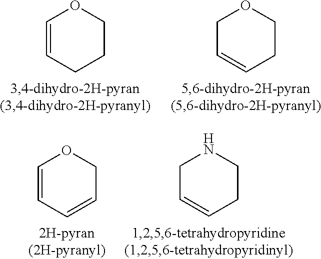 [SMILES: C1=COCCC1 ; C1=CCOCC1 ; C1=CCOC=C1 ; C1=CCNCC1]

Other illustrative examples of heterocyclo or heterocycloalkyl groups, also referred to as non-aromatic heterocycles, include:

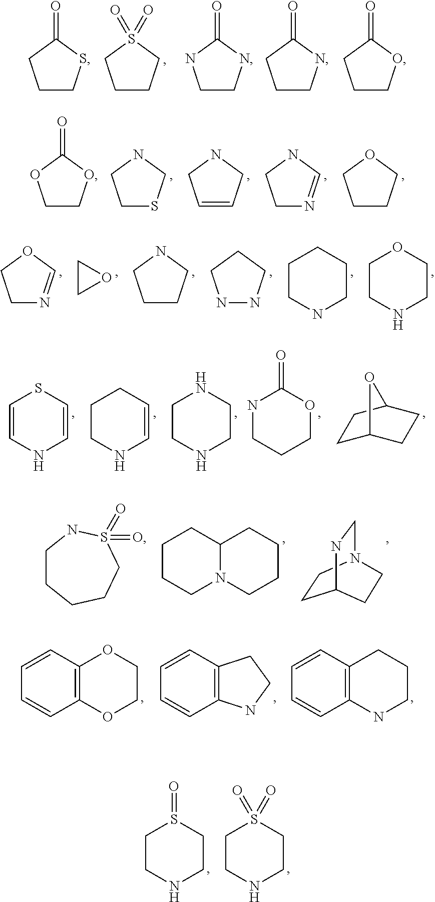

or the like.

The term heteroalicyclic also includes all ring forms of the carbohydrates, including but not limited to the monosaccharides, the disaccharides and the oligosaccharides.

The term “halo” or, alternatively, “halogen” means fluoro, chloro, bromo and iodo.

The terms “haloalkyl” and “haloalkoxy” include alkyl and alkoxy structures that are substituted with one or more halogens. In embodiments, where more than one halogen is included in the group, the halogens are the same or they are different. The terms “fluoroalkyl” and “fluoroalkoxy” include haloalkyl and haloalkoxy groups, respectively, in which the halo is fluorine.

The term “heteroalkyl” include optionally substituted alkyl, alkenyl and alkynyl radicals which have one or more skeletal chain atoms selected from an atom other than carbon, e.g., oxygen, nitrogen, sulfur, phosphorus, silicon, or combinations thereof. In certain embodiments, the heteroatom(s) is placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In some embodiments, up to two heteroatoms are consecutive, such as, by way of example, —CH₂—NH—OCH₃ and —CH₂—O—Si(CH₃)₃.

A “cyano” group refers to a CN group.

An “isocyanato” group refers to a NCO group.

A “thiocyanato” group refers to a CNS group.

An “isothiocyanato” group refers to a NCS group.

“Alkoyloxy” refers to a RC(═O)O— group.

“Alkoyl” refers to a RC(═O)— group.

“Isosteres” of a chemical group are chemical groups that have different molecular formulae but exhibit the same or similar properties. For example, tetrazole is an isostere of carboxylic acid because it mimics the properties of carboxylic acid even though they both have very different molecular formulae. Tetrazole is one of many possible isosteric replacements for carboxylic acid. Other carboxylic acid isosteres contemplated include SO₃H, —SO₂NHR₄, —P(O)(OR₄)₂, —P(O)(R₄)(OR₄), —CON(R₄)₂, —CONHNHSO₂R₄, —CONHSO₂R₄, —B(OR₅)₂, —C(R₄)₂B(OR₅)₂, and —CON(R₄)C(R₄)₂B(OR₅)₂; wherein each R₄ is independently H, OH, substituted or unsubstituted alkyl, substituted or unsubstituted heteroaryl, or substituted or unsubstituted aryl; and R₅ is H or C₁-C₆alkyl. In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of preferred carbocyclic and heterocyclic isosteres contemplated.

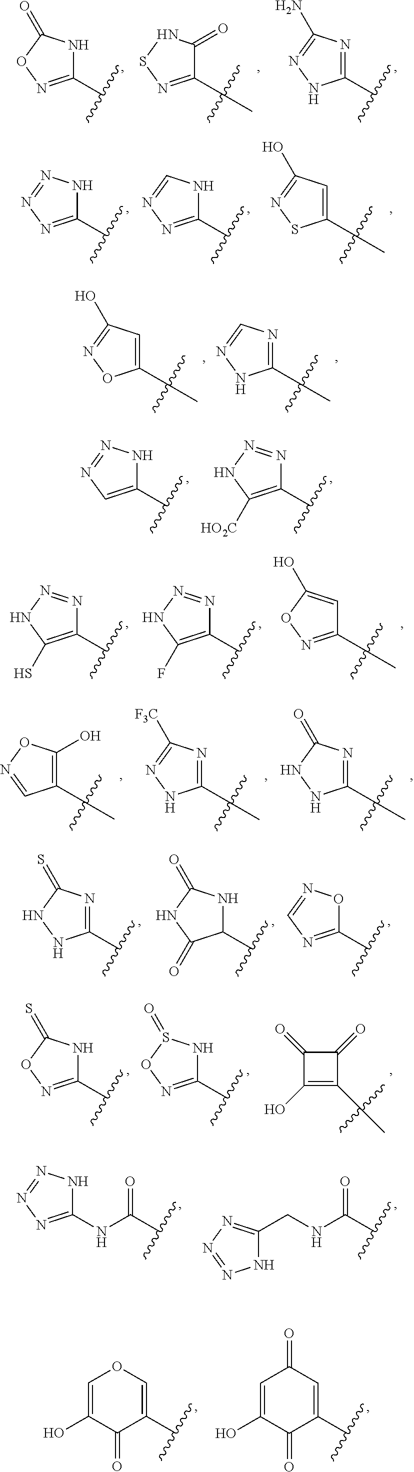

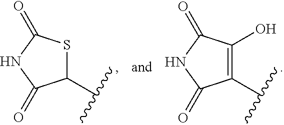 [SMILES: *C1SC(=O)NC1=O ; *C1=C(O)C(=O)NC1=O]

It is also contemplated that when chemical substituents are added to a carboxylic acid isostere then the inventive compound retains the properties of a carboxylic acid isostere. The present invention contemplates that when a carboxylic acid isostere is optionally substituted, then the substitution cannot eliminate the carboxylic acid isosteric properties of the inventive compound. It is contemplated that the placement of one or more substituents upon a carbocyclic or heterocyclic carboxylic acid isostere is not a substitution at one or more atom(s) which maintain(s) or is/are integral to the carboxylic acid isosteric properties of the compound, if such substituent(s) would destroy the carboxylic acid isosteric properties of the compound.

Other carboxylic acid isosteres not specifically exemplified or described in this specification are also contemplated by the present invention.

### CSE Inhibitors

In the following description of CSE inhibitory compounds suitable for use in the methods described herein, definitions of referred-to standard chemistry terms may be found in reference works (if not otherwise defined herein), including Carey and Sundberg “Advanced Organic Chemistry 4th Ed.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the ordinary skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients.

Described herein are compounds of any of Formula (I), (II), (III), (IV), (V), (VI), (VII), (VIII), (IX), or (X). Also described herein are pharmaceutically acceptable salts, pharmaceutically acceptable solvates, and pharmaceutically acceptable prodrugs of such compounds. Pharmaceutical compositions that include at least one such compound or a pharmaceutically acceptable salt, pharmaceutically acceptable solvate, or pharmaceutically acceptable prodrug of such compound, are provided. In certain embodiments, isomers and chemically protected forms of compounds having a structure represented by any of Formula (I), (II), (III), (IV), (V), (VI), (VII), (VIII), (IX), or (X) are also provided.

In one aspect is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (I) having the structure:

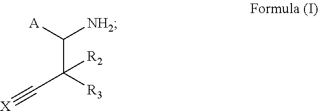

wherein:
A is a carboxylic acid isostere;

### X is CR₁, or N;

R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl; and
R₂ and R₃ are each independently H, substituted or unsubstituted alkyl, or substituted or unsubstituted heteroalkyl; or R₂ and R₃ together with the carbon to which they are attached form a cycloalkyl or heterocycloalkyl ring;
or a pharmaceutically acceptable salt, solvate, or prodrug thereof.

In another aspect is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (II) having the structure:

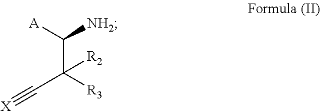

wherein:
A is a carboxylic acid isostere;

### X is CR₁, or N;

R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl; and
R₂ and R₃ are each independently H, substituted or unsubstituted alkyl, or substituted or unsubstituted heteroalkyl; or R₂ and R₃ together with the carbon to which they are attached form a cycloalkyl or heterocycloalkyl ring;
or a pharmaceutically acceptable salt, solvate, or prodrug thereof.

In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (I) or (II) wherein A is a carboxylic acid isostere selected from:

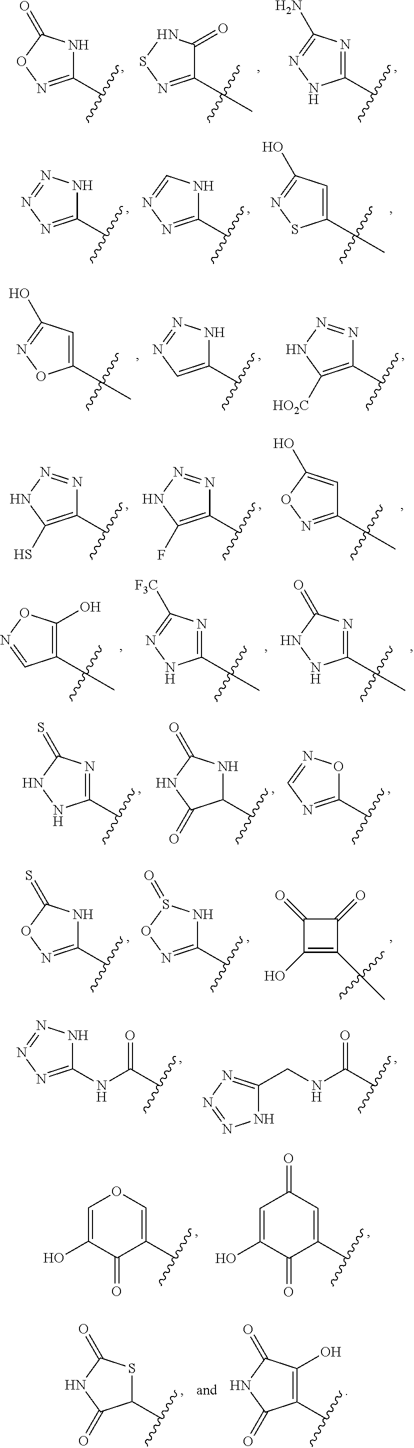

In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (I) or (II) wherein A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —B(OR₅)₂, —C(R₄)₂B(OR₅)₂, and —CON(R₄)C(R₄)₂B(OR₅)₂; wherein each R₄ is independently H, OH, substituted or unsubstituted alkyl, or substituted or unsubstituted aryl; and R₅ is H or C₁-C₆alkyl.

In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (I) or (II) wherein A is a carboxylic acid isostere selected from —SO₃H, —SO₂NHR₄, —P(O)(OR₄)₂, —P(O)(R₄)(OR₄), —C(O)R₄, —CON(R₄)₂, —CONHNHSO₂R₄, —CONHSO₂R₄, —B(OR₅)₂, —C(R₄)₂B(OR₅)₂, and —CON(R₄)C(R₄)₂B(OR₅)₂; wherein each R₄ is independently H, OH, substituted or unsubstituted alkyl, substituted or unsubstituted heteroaryl, or substituted or unsubstituted aryl; and R₅ is H or C₁-C₆alkyl.

In further embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (I) or (II) wherein X is CR₁. In yet further embodiments, X is CR₁; and R₁ is H, substituted or unsubstituted alkyl, or substituted or unsubstituted heteroalkyl. In some embodiments, X is CR₁; and R₁ is H. In some embodiments, X is CR₁; and R₁ is substituted or unsubstituted alkyl. In some embodiments, X is CR₁; and R₁ is CH₃. In yet further embodiments, X is CR₁; and R₁ is substituted or unsubstituted heteroalkyl. In other embodiments, X is CR₁; and R₁ is substituted or unsubstituted heterocycloalkyl. In some embodiments, X is CR₁; and R₁ is substituted or unsubstituted aryl. In other embodiments, X is CR₁; and R₁ is substituted or unsubstituted heteroaryl.

In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (I) or (II) wherein X is N.

In any of the aforementioned embodiments is a method for treating chemotherapy-induced peripheral neuropathy, or human immunodeficiency virus-associated sensory neuropathy, or symptoms thereof, comprising administering to an individual in need thereof a therapeutically effective amount of Formula (I) or (II) wherein R₂ and R₃ are each independently H, substituted or unsubstituted alkyl, or substituted or unsubstituted heteroalkyl. In any of the aforementioned embodiments, R₂ and R₃ are each H. In any of the aforementioned embodiments, R₂ and R₃ are each independently substituted or unsubstituted alkyl. In any of the aforementioned embodiments, R₂ and R₃ are each independently substituted or unsubstituted heteroalkyl. In any of the aforementioned embodiments, A is

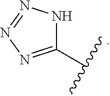 [SMILES: *c1nnn[nH]1]

In any of the aforementioned embodiments, A is

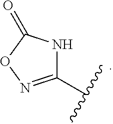 [SMILES: *c1noc(=O)[nH]1]

In any of the aforementioned embodiments, A is

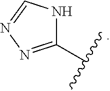 [SMILES: *c1nnc[nH]1]

In another aspect is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (IIa) having the structure:

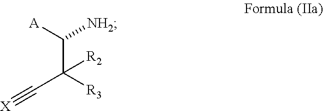

wherein:
A is a carboxylic acid isostere;

### X is CR₁, or N;

R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl;
R₂ and R₃ are each independently H, substituted or unsubstituted alkyl, or substituted or unsubstituted heteroalkyl; or R₂ and R₃ together with the carbon to which they are attached form a cycloalkyl or heterocycloalkyl ring;
or a pharmaceutically acceptable salt, solvate, or prodrug thereof.

In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (I), (II), or (IIa) wherein A is a carboxylic acid isostere selected from:

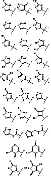

In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (I), (II), or (IIa) wherein A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —B(OR₅)₂, —C(R₄)₂B(OR₅)₂, and —CON(R₄)C(R₄)₂B(OR₅)₂; wherein each R₄ is independently H, OH, substituted or unsubstituted alkyl, or substituted or unsubstituted aryl; and R₅ is H or C₁-C₆alkyl.

In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (I), (II), or (IIa) wherein A is a carboxylic acid isostere selected from —SO₃H, —SO₂NHR₄, —P(O)(OR₄)₂, —P(O)(R₄)(OR₄), —C(O)R₄, —CON(R₄)₂, —CONHNHSO₂R₄, —CONHSO₂R₄, —B(OR₅)₂, —C(R₄)₂B(OR₅)₂, and —CON(R₄)C(R₄)₂B(OR₅)₂; wherein each R₄ is independently H, OH, substituted or unsubstituted alkyl, substituted or unsubstituted heteroaryl, or substituted or unsubstituted aryl; and R₅ is H or C₁-C₆alkyl.

In further embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (I), (II), or (IIa) wherein X is CR₁. In yet further embodiments, X is CR₁; and R₁ is H, substituted or unsubstituted alkyl, or substituted or unsubstituted heteroalkyl. In some embodiments, X is CR₁; and R₁ is H. In some embodiments, X is CR₁; and R₁ is substituted or unsubstituted alkyl. In some embodiments, X is CR₁; and R₁ is CH₃. In yet further embodiments, X is CR₁; and R₁ is substituted or unsubstituted heteroalkyl. In other embodiments, X is CR₁; and R₁ is substituted or unsubstituted heterocycloalkyl. In some embodiments, X is CR₁; and R₁ is substituted or unsubstituted aryl. In other embodiments, X is CR₁; and R₁ is substituted or unsubstituted heteroaryl.

In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (I), (II), or (IIa) wherein X is N.

In another aspect is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (III) having the structure:

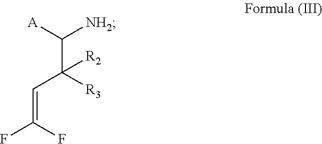

wherein:
A is a carboxylic acid isostere;
R₂ and R₃ are each independently H, substituted or unsubstituted alkyl, or substituted or unsubstituted heteroalkyl; or R₂ and R₃ together with the carbon to which they are attached form a cycloalkyl or heterocycloalkyl ring;
or a pharmaceutically acceptable salt, solvate, or prodrug thereof.

In another aspect is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (IV) having the structure:

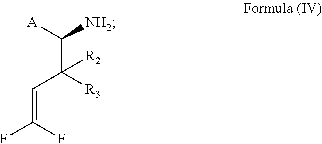

wherein:
A is a carboxylic acid isostere;
R₂ and R₃ are each independently H, substituted or unsubstituted alkyl, or substituted or unsubstituted heteroalkyl; or R₂ and R₃ together with the carbon to which they are attached form a cycloalkyl or heterocycloalkyl ring;
or a pharmaceutically acceptable salt, solvate, or prodrug thereof.

In another aspect is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (IVa) having the structure:

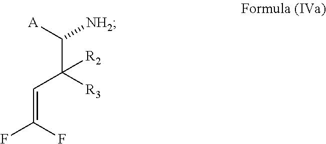

wherein:
A is a carboxylic acid isostere;
R₂ and R₃ are each independently H, substituted or unsubstituted alkyl, or substituted or unsubstituted heteroalkyl; or R₂ and R₃ together with the carbon to which they are attached form a cycloalkyl or heterocycloalkyl ring;
or a pharmaceutically acceptable salt, solvate, or prodrug thereof.

In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (III), (IV), or (IVa) wherein A is a carboxylic acid isostere selected from:

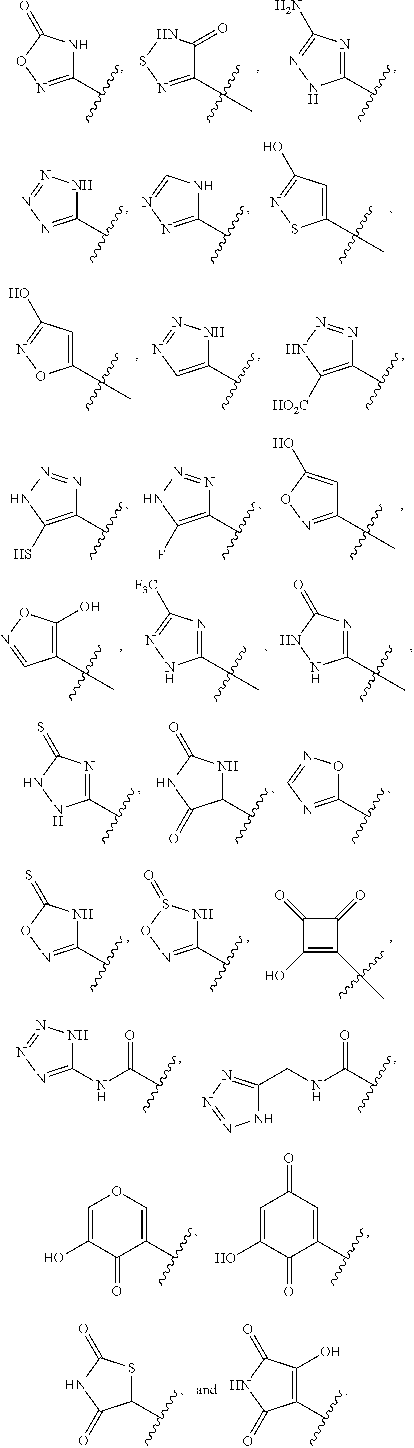

In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (III), (IV), or (IVa) wherein A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —B(OR₅)₂, —C(R₄)₂B(OR₅)₂, and —CON(R₄)C(R₄)₂B(OR₅)₂; wherein each R₄ is independently H, OH, substituted or unsubstituted alkyl, or substituted or unsubstituted aryl; and R₅ is H or C₁-C₆alkyl.

In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (III), (IV), or (IVa) wherein A is a carboxylic acid isostere selected from —SO₃H, —SO₂NHR₄, —P(O)(OR₄)₂, —P(O)(R₄)(OR₄), —C(O)R₄, —CON(R₄)₂, —CONHNHSO₂R₄, —CONHSO₂R₄, —B(OR₅)₂, —C(R₄)₂B(OR₅)₂, and —CON(R₄)C(R₄)₂B(OR₅)₂; wherein each R₄ is independently H, OH, substituted or unsubstituted alkyl, substituted or unsubstituted heteroaryl, or substituted or unsubstituted aryl; and R₅ is H or C₁-C₆alkyl.

In any of the aforementioned embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (I), (II), (III), or (IV) wherein R₂ and R₃ are each independently H, substituted or unsubstituted alkyl, or substituted or unsubstituted heteroalkyl. In any of the aforementioned embodiments, R₂ and R₃ are each H. In any of the aforementioned embodiments, R₂ and R₃ are each independently substituted or unsubstituted alkyl. In any of the aforementioned embodiments, R₂ and R₃ are each independently substituted or unsubstituted heteroalkyl.

In any of the aforementioned embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (I), (II), (III), or (IV) wherein A is

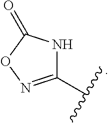 [SMILES: *c1noc(=O)[nH]1]

In any of the aforementioned embodiments, A is

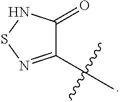 [SMILES: *c1ns[nH]c1=O]

In any of the aforementioned embodiments, A is

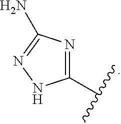 [SMILES: *c1nc(N)n[nH]1]

In any of the aforementioned embodiments, A is

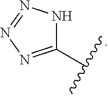 [SMILES: *c1nnn[nH]1]

In any of the aforementioned embodiments, A is

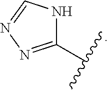 [SMILES: *c1nnc[nH]1]

In any of the aforementioned embodiments, A is

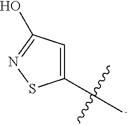 [SMILES: *c1cc(O)ns1]

In any of the aforementioned embodiments, A is

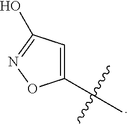 [SMILES: *c1cc(O)no1]

In any of the aforementioned embodiments, A is

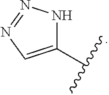 [SMILES: *c1cnn[nH]1]

In any of the aforementioned embodiments, A is

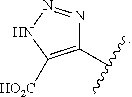 [SMILES: *c1nn[nH]c1C(=O)O]

In any of the aforementioned embodiments, A is

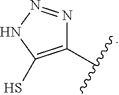 [SMILES: *c1nn[nH]c1S]

In any of the aforementioned embodiments, A is

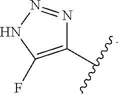 [SMILES: *c1nn[nH]c1F]

In any of the aforementioned embodiments, A is

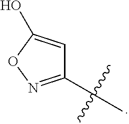 [SMILES: *c1cc(O)on1]

In any of the aforementioned embodiments, A is

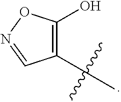 [SMILES: *c1cnoc1O]

In any of the aforementioned embodiments, A is

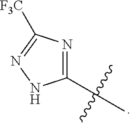 [SMILES: *c1nc(C(F)(F)F)n[nH]1]

In any of the aforementioned embodiments, A is

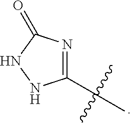 [SMILES: *c1nc(=O)[nH][nH]1]

In any of the aforementioned embodiments, A is

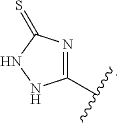 [SMILES: *c1nc(=S)[nH][nH]1]

In any of the aforementioned embodiments, A is

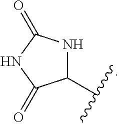 [SMILES: *C1NC(=O)NC1=O]

In any of the aforementioned embodiments, A is

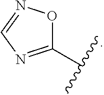 [SMILES: *c1ncno1]

In any of the aforementioned embodiments, A is

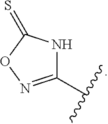 [SMILES: *c1noc(=S)[nH]1]

In any of the aforementioned embodiments, A is

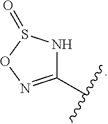 [SMILES: *C1=NOS(=O)N1]

In any of the aforementioned embodiments, A is

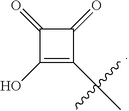 [SMILES: *c1c(O)c(=O)c1=O]

In any of the aforementioned embodiments, A is

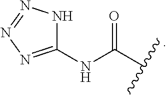 [SMILES: *C(=O)Nc1nnn[nH]1]

In any of the aforementioned embodiments, A is

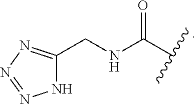 [SMILES: *C(=O)NCc1nnn[nH]1]

In any of the aforementioned embodiments, A is

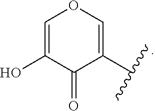 [SMILES: *c1cocc(O)c1=O]

In any of the aforementioned embodiments, A is

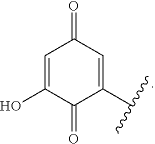 [SMILES: *C1=CC(=O)C=C(O)C1=O]

In any of the aforementioned embodiments, A is

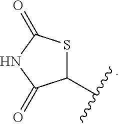 [SMILES: *C1SC(=O)NC1=O]

In any of the aforementioned embodiments, A is

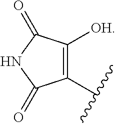 [SMILES: *C1=C(O)C(=O)NC1=O]

In another embodiment is a method for treating chemotherapy-induced peripheral neuropathy, or human immunodeficiency virus-associated sensory neuropathy, or symptoms thereof, comprising administering to an individual in need thereof a therapeutically effective amount of a compound having the structure:

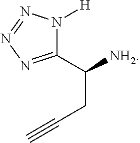

In another embodiment is a method for treating chemotherapy-induced peripheral neuropathy, or human immunodeficiency virus-associated sensory neuropathy, or symptoms thereof, comprising administering to an individual in need thereof a therapeutically effective amount of a compound having the structure:

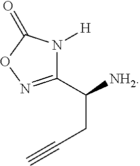

In another embodiment is a method for treating chemotherapy-induced peripheral neuropathy, or human immunodeficiency virus-associated sensory neuropathy, or symptoms thereof, comprising administering to an individual in need thereof a therapeutically effective amount of a compound having the structure:

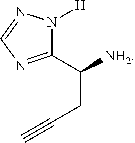

In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound selected from:

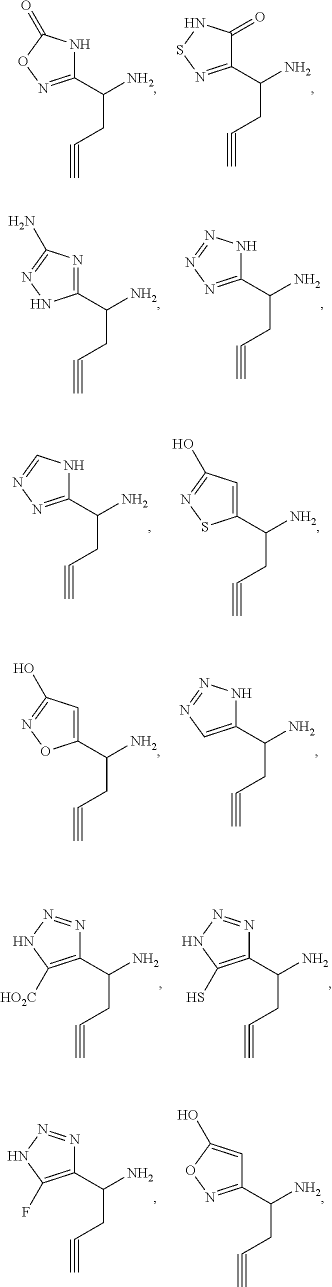

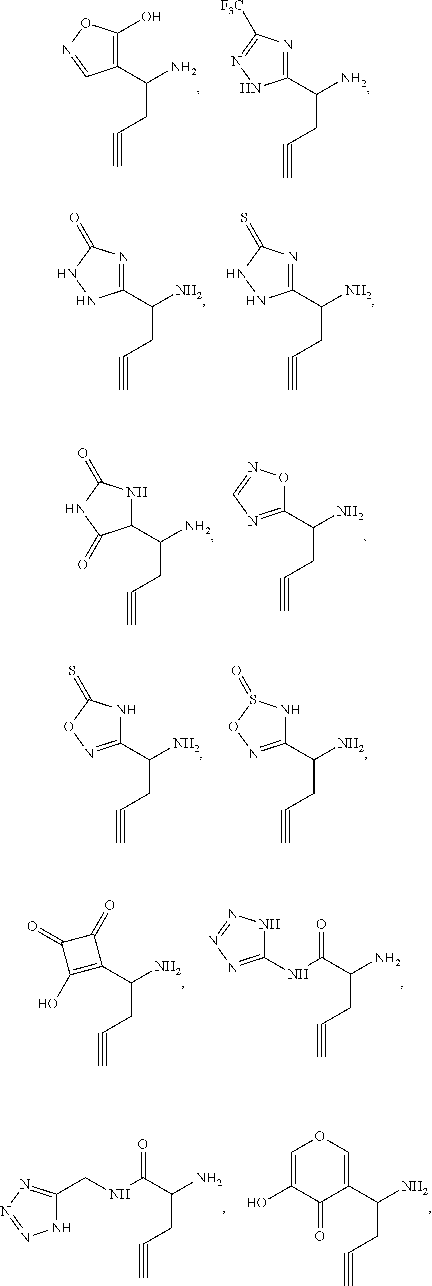

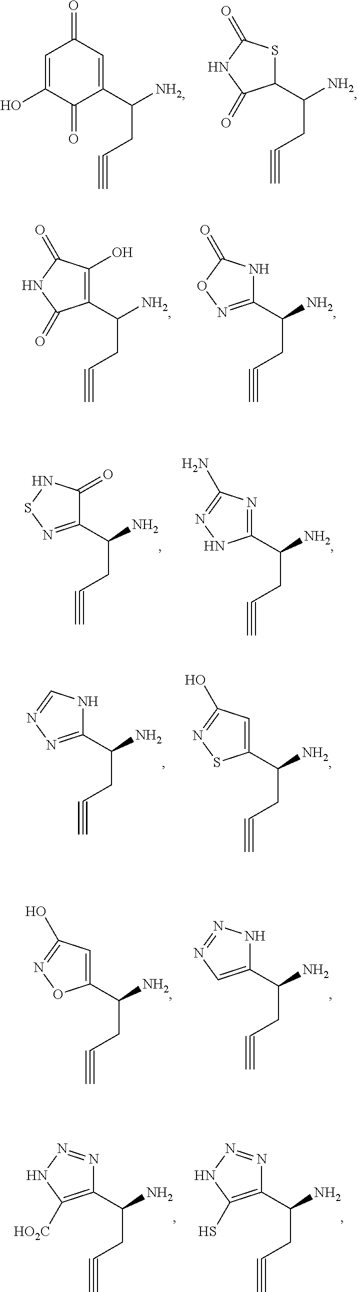

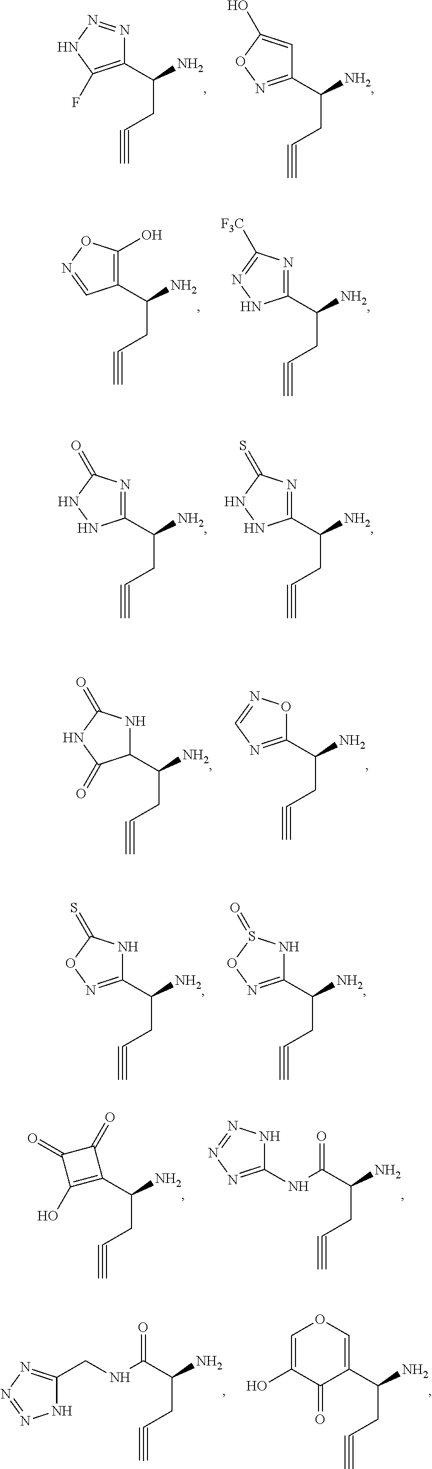

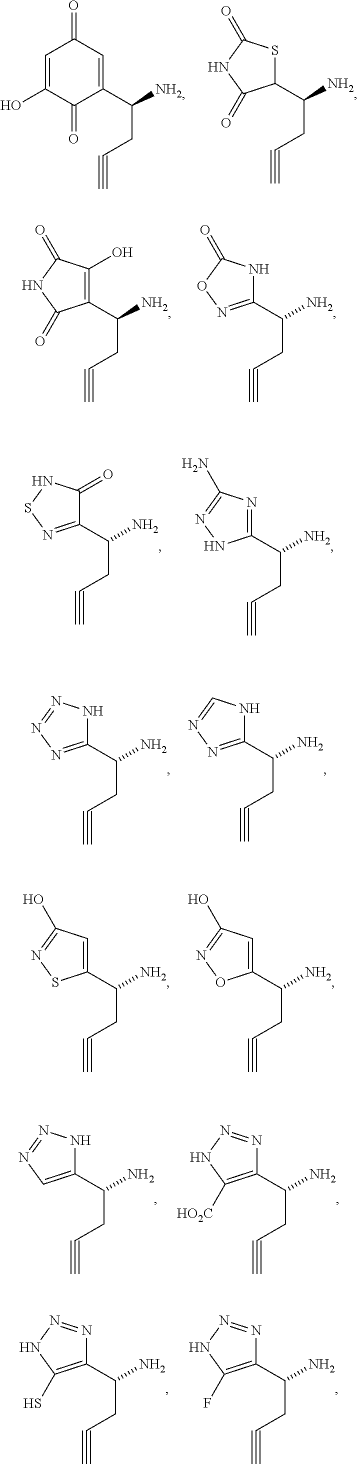

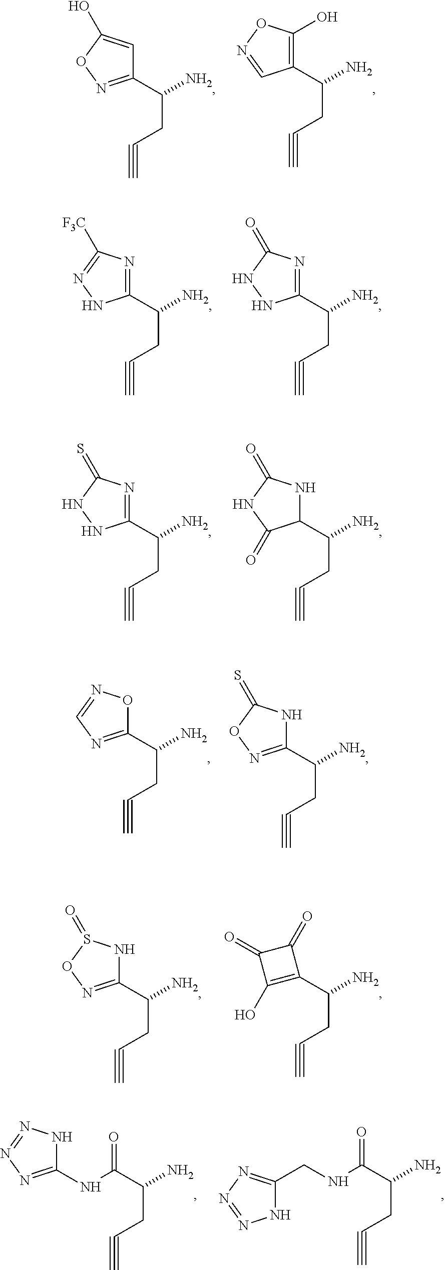

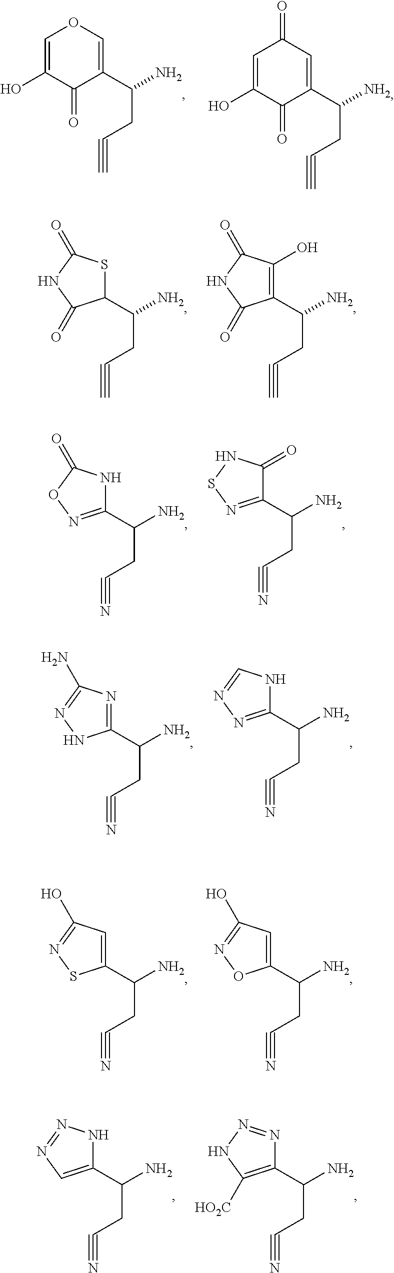

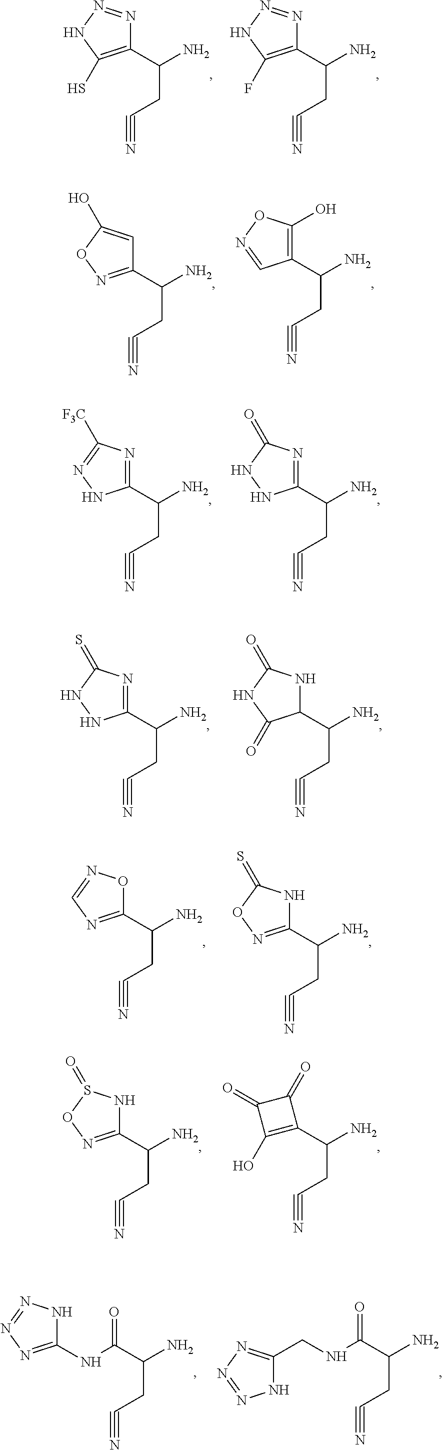

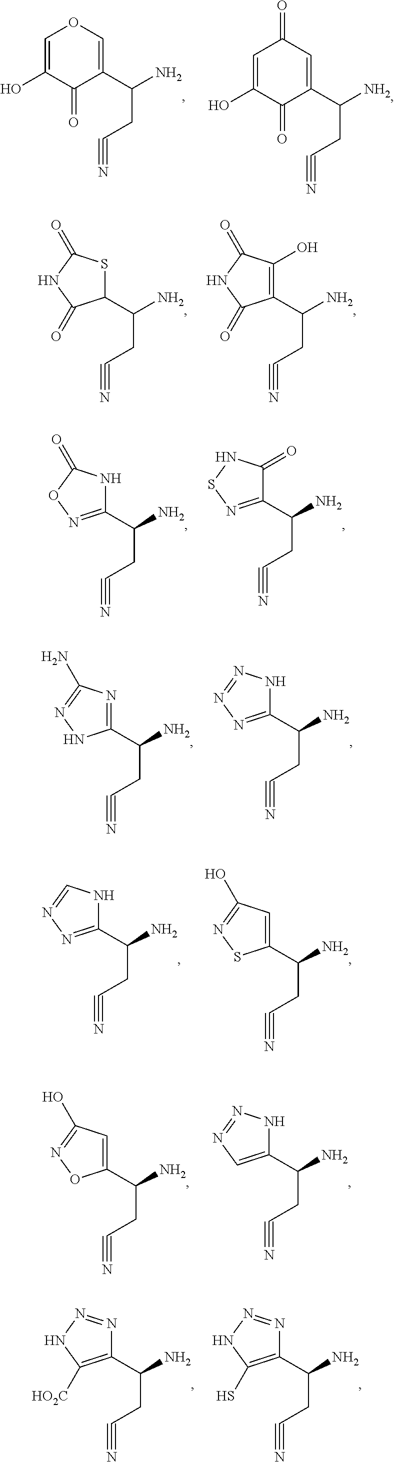

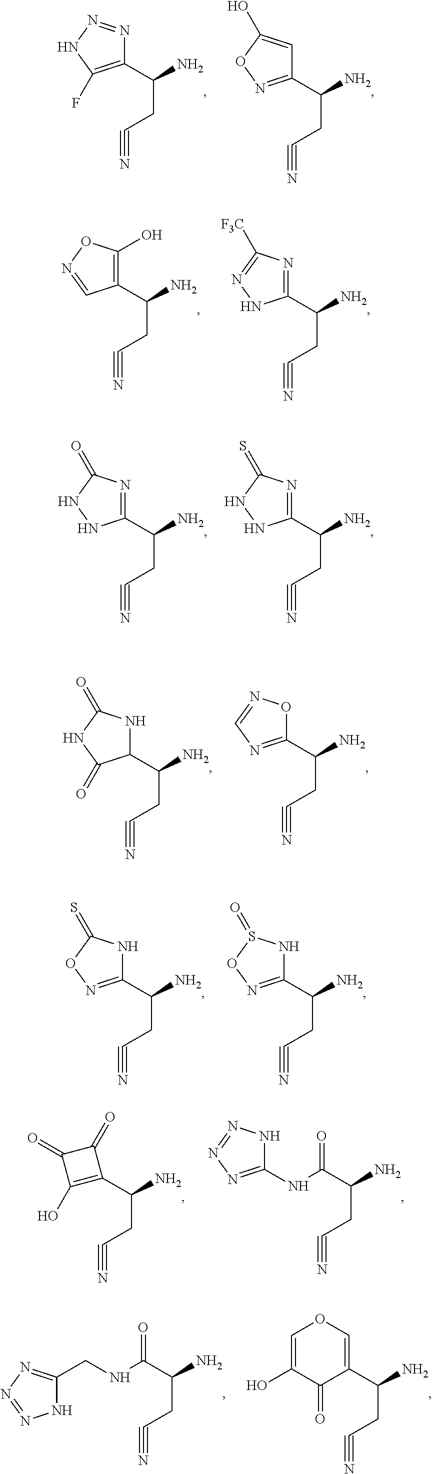

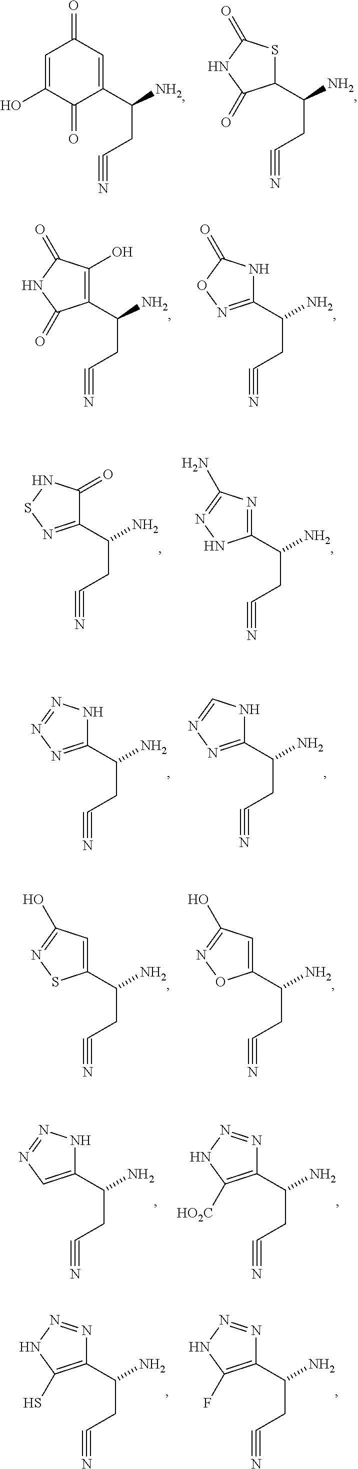

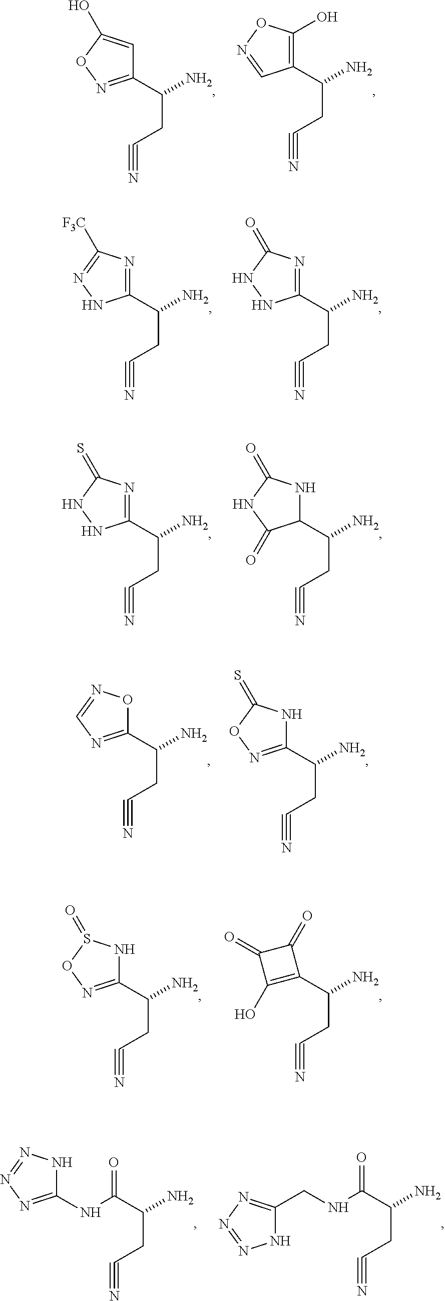

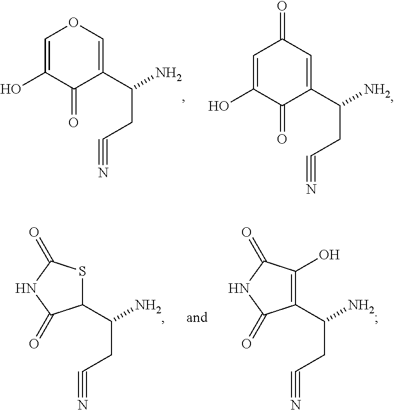 [SMILES: N#CC[C@@H](N)c1cocc(O)c1=O ; N#CC[C@@H](N)C1=CC(=O)C=C(O)C1=O ; N#CC[C@@H](N)C1SC(=O)NC1=O ; N#CC[C@@H](N)C1=C(O)C(=O)NC1=O]

or a pharmaceutically acceptable salt, solvate, or prodrug thereof.

In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound selected from:

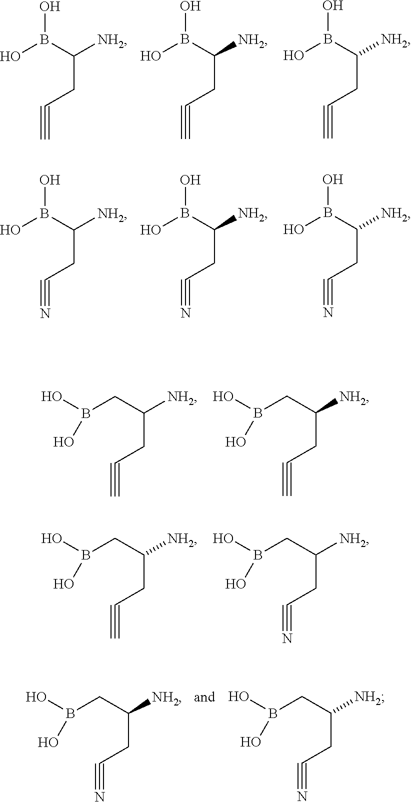

or a pharmaceutically acceptable salt, solvate, or prodrug thereof.

In another aspect is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (V) having the structure:

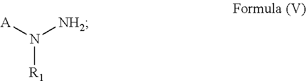

wherein:

A is a carboxylic acid isostere;

R₁ is substituted or unsubstituted C₃-C₆alkyl, substituted or unsubstituted heteroalkyl, substituted or unsubstituted heterocycloalkyl, substituted or unsubstituted aryl, or substituted or unsubstituted heteroaryl; or a pharmaceutically acceptable salt, solvate, or prodrug thereof.

In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (V) wherein A is a carboxylic acid isostere selected from:

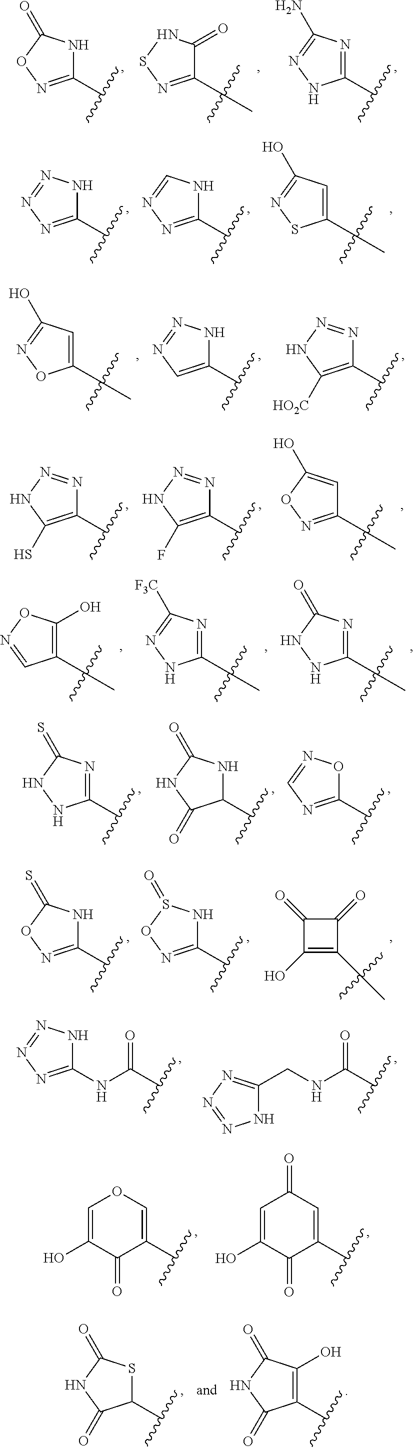

In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (V) wherein A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂; wherein each R₄ is independently H, OH, substituted or unsubstituted alkyl, or substituted or unsubstituted aryl; and R₅ is H or C₁-C₆alkyl.

In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (V) wherein A is a carboxylic acid isostere selected from —SO₃H, —SO₂NHR₄, —P(O)(OR₄)₂, —P(O)(R₄)(OR₄), —C(O)R₄, —CON(R₄)₂, —CONHNHSO₂R₄, —CONHSO₂R₄, —C(R₄)₂B(OR₅)₂, and —CON(R₄)C(R₄)₂B(OR₅)₂; wherein each R₄ is independently H, OH, substituted or unsubstituted alkyl, substituted or unsubstituted heteroaryl, or substituted or unsubstituted aryl; and R₅ is H or C₁-C₆alkyl.

In another embodiment is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (V) wherein R₁ is H, substituted or unsubstituted C₃-C₆alkyl, substituted or unsubstituted heteroalkyl, substituted or unsubstituted heterocycloalkyl, substituted or unsubstituted aryl, or substituted or unsubstituted heteroaryl. In some embodiments, R₁ is substituted or unsubstituted C₃-C₆alkyl. In further embodiments, R₁ is propyl. In further embodiments, R₁ is butyl. In some embodiments, R₁ is substituted or unsubstituted heteroalkyl. In some embodiments, R₁ is substituted or unsubstituted heterocycloalkyl. In some embodiments, R₁ is substituted or unsubstituted aryl. In some embodiments, R₁ is substituted or unsubstituted heteroaryl.

In another aspect is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VI) having the structure:

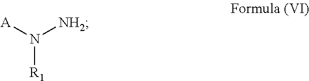

wherein:

R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl;

A is selected from

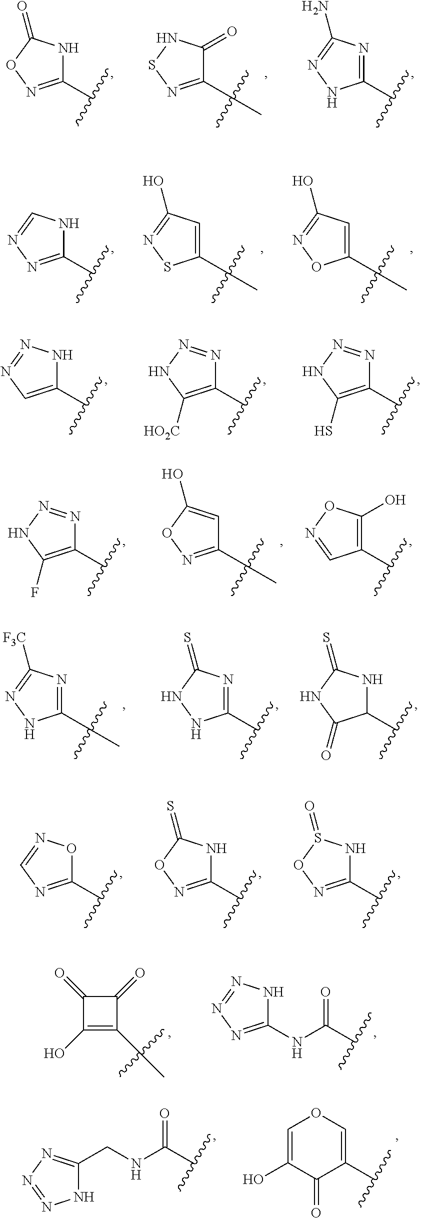

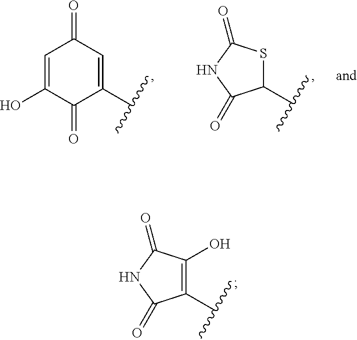 [SMILES: *C1=CC(=O)C=C(O)C1=O ; *C1SC(=O)NC1=O ; *C1=C(O)C(=O)NC1=O]

or
a pharmaceutically acceptable salt, solvate, or prodrug thereof.

In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VI) wherein A is selected from

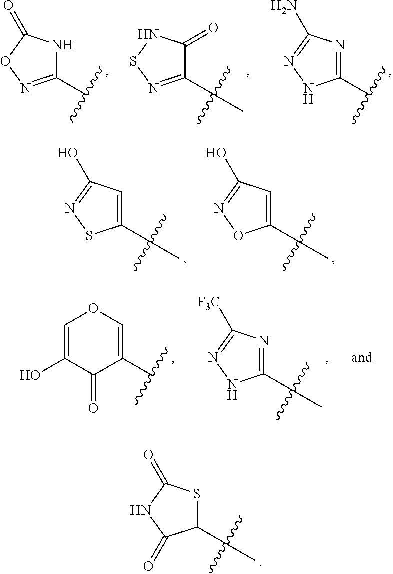

In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VI) wherein A is

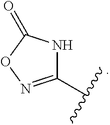 [SMILES: *c1noc(=O)[nH]1]

In some embodiments, A is

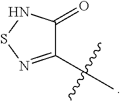 [SMILES: *c1ns[nH]c1=O]

In some embodiments, A is

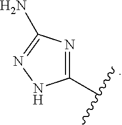 [SMILES: *c1nc(N)n[nH]1]

In some embodiments, A is

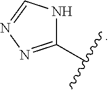 [SMILES: *c1nnc[nH]1]

In some embodiments, A is

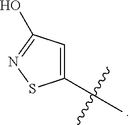 [SMILES: *c1cc(O)ns1]

In some embodiments, A is

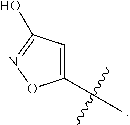 [SMILES: *c1cc(O)no1]

In some embodiments, A is

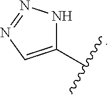 [SMILES: *c1cnn[nH]1]

In some embodiments, A is

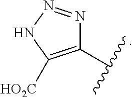 [SMILES: *c1nn[nH]c1C(=O)O]

In some embodiments, A is

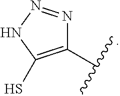 [SMILES: *c1nn[nH]c1S]

In some embodiments, A is

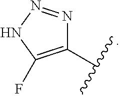 [SMILES: *c1nn[nH]c1F]

In some embodiments, A is

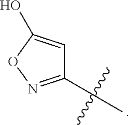 [SMILES: *c1cc(O)on1]

In some embodiments, A is

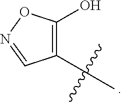 [SMILES: *c1cnoc1O]

In some embodiments, A is

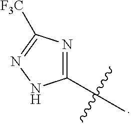 [SMILES: *c1nc(C(F)(F)F)n[nH]1]

In some embodiments, A is

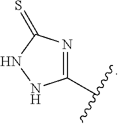 [SMILES: *c1nc(=S)[nH][nH]1]

In some embodiments, A is

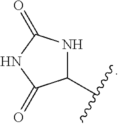 [SMILES: *C1NC(=O)NC1=O]

In some embodiments, A is

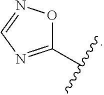 [SMILES: *c1ncno1]

In some embodiments, A is

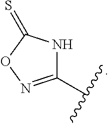 [SMILES: *c1noc(=S)[nH]1]

In some embodiments, A is

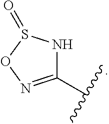 [SMILES: *C1=NOS(=O)N1]

In some embodiments, A is

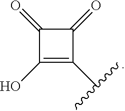 [SMILES: *c1c(O)c(=O)c1=O]

In some embodiments, A is

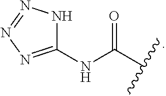 [SMILES: *C(=O)Nc1nnn[nH]1]

In some embodiments, A is

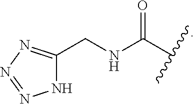 [SMILES: *C(=O)NCc1nnn[nH]1]

In some embodiments, A is

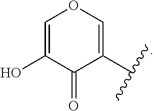 [SMILES: *c1cocc(O)c1=O]

In some embodiments, A is

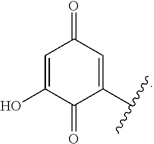 [SMILES: *C1=CC(=O)C=C(O)C1=O]

In some embodiments, A is

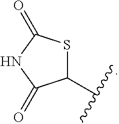 [SMILES: *C1SC(=O)NC1=O]

In some embodiments, A is

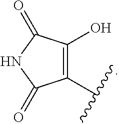 [SMILES: *C1=C(O)C(=O)NC1=O]

In another embodiment is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VI) wherein R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl. In some embodiments, R₁ is H. In some embodiments, R₁ is substituted or unsubstituted alkyl. In further embodiments, R₁ is methyl. In further embodiments, R₁ is ethyl. In further embodiments, R₁ is propyl. In further embodiments, R₁ is butyl. In some embodiments, R₁ is substituted or unsubstituted heteroalkyl. In some embodiments, R₁ is substituted or unsubstituted heterocycloalkyl. In some embodiments, R₁ is substituted or unsubstituted aryl. In some embodiments, R₁ is substituted or unsubstituted heteroaryl.

In another aspect is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VII) having the structure:

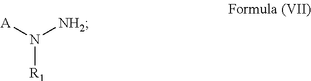

wherein:

R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl;

A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂; wherein each R₄ is independently H, OH, substituted or unsubstituted alkyl, or substituted or unsubstituted aryl; and R₅ is H or C₁-C₆alkyl; or a pharmaceutically acceptable salt, solvate, or prodrug thereof.

In another embodiment is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VII) wherein R₁ is H. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VII) wherein R₁ is substituted or unsubstituted alkyl. In further embodiments, R₁ is methyl. In further embodiments, R₁ is ethyl. In further embodiments, R₁ is propyl. In further embodiments, R₁ is butyl. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VII) wherein R₁ is substituted or unsubstituted heteroalkyl. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VII) wherein R₁ is substituted or unsubstituted heterocycloalkyl. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VII) wherein R₁ is substituted or unsubstituted aryl. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VII) wherein R₁ is substituted or unsubstituted heteroaryl.

In another embodiment is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VII) wherein R₁ is H, and A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂. In another embodiment is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VII) wherein R₁ is substituted or unsubstituted alkyl, and A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂. In further embodiments, R₁ is methyl and A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂. In further embodiments, R₁ is ethyl and A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VII) wherein R₁ is substituted or unsubstituted heteroalkyl and A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VII) wherein R₁ is substituted or unsubstituted heterocycloalkyl and A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VII) wherein R₁ is substituted or unsubstituted aryl and A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VII) wherein R₁ is substituted or unsubstituted heteroaryl and A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂. In any of the aforementioned embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VII) wherein A is —SO₃H. In any of the aforementioned embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VII) wherein A is —SO₂NHR₄. In any of the aforementioned embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VII) wherein A is —P(O)(OR₄)₂. In any of the aforementioned embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VII) wherein A is —P(O)(R₄)(OR₄). In any of the aforementioned embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VII) wherein A is —CON(R₄)₂. In any of the aforementioned embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VII) wherein A is —CONHNHSO₂R₄. In any of the aforementioned embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VII) wherein A is —CONHSO₂R₄. In any of the aforementioned embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VII) wherein A is —C(R₄)₂B(OR₅)₂. In any of the aforementioned embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VII) wherein A is —CON(R₄)C(R₄)₂B(OR₅)₂.

In another aspect is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VIII) having the structure:

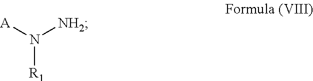

wherein:

A is

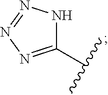 [SMILES: *c1nnn[nH]1]

R₁ is substituted or unsubstituted C₂-C₆alkyl, substituted or unsubstituted heteroalkyl, substituted or unsubstituted heterocycloalkyl, substituted or unsubstituted aryl, or substituted or unsubstituted heteroaryl; or a pharmaceutically acceptable salt, solvate, or prodrug thereof.

In another embodiment is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VIII) wherein R₁ is substituted or unsubstituted C₂-C₆alkyl, substituted or unsubstituted heteroalkyl, substituted or unsubstituted heterocycloalkyl, substituted or unsubstituted aryl, or substituted or unsubstituted heteroaryl. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VIII) wherein R₁ is substituted or unsubstituted C₂-C₆alkyl or substituted or unsubstituted heteroalkyl. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VIII) wherein R₁ is substituted or unsubstituted C₂-C₆alkyl. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VIII) wherein R₁ is substituted or unsubstituted —CH₂CH₃. In further embodiments, R₁ is propyl. In further embodiments, R₁ is butyl. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VIII) wherein R₁ is substituted or unsubstituted heteroalkyl. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VIII) wherein R₁ is substituted or unsubstituted heterocycloalkyl. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VIII) wherein R₁ is substituted or unsubstituted aryl. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (VIII) wherein R₁ is substituted or unsubstituted heteroaryl.

In another aspect is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (IX) having the structure:

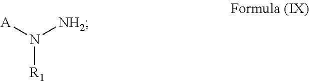

wherein:

A is

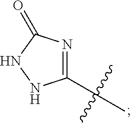 [SMILES: *c1nc(=O)[nH][nH]1]

R₁ is H, substituted or unsubstituted C₃-C₆alkyl, substituted or unsubstituted heteroalkyl, substituted or unsubstituted heterocycloalkyl, substituted or unsubstituted aryl, or substituted or unsubstituted heteroaryl; or a pharmaceutically acceptable salt, solvate, or prodrug thereof.

In another embodiment is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (IX) wherein R₁ is substituted or unsubstituted C₃-C₆alkyl, substituted or unsubstituted heteroalkyl, substituted or unsubstituted heterocycloalkyl, substituted or unsubstituted aryl, or substituted or unsubstituted heteroaryl. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (IX) wherein R₁ is H. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (IX) wherein R₁ is substituted or unsubstituted C₃-C₆alkyl. In further embodiments, R₁ is propyl. In further embodiments, R₁ is isopropyl. In further embodiments, R₁ is butyl. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (IX) wherein R₁ is substituted or unsubstituted heteroalkyl. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (IX) wherein R₁ is substituted or unsubstituted heterocycloalkyl. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (IX) wherein R₁ is substituted or unsubstituted aryl. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (IX) wherein R₁ is substituted or unsubstituted heteroaryl.

In another aspect is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (X) having the structure:

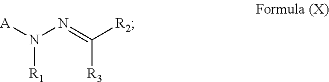

wherein:

A is a carboxylic acid isostere; R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl;

R₂ is substituted or unsubstituted alkyl, substituted or unsubstituted heteroalkyl, substituted or unsubstituted heterocycloalkyl, substituted or unsubstituted aryl, substituted or unsubstituted heteroaryl, or —CH₂C(O) (substituted or unsubstituted aryl);

R₃ is H, or substituted or unsubstituted alkyl; or

R₂ and R₃ together with the carbon atom to which they are attached form a cycloalkyl or heterocycloalkyl ring; or a pharmaceutically acceptable salt, solvate, or prodrug thereof.

In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (X) wherein A is a carboxylic acid isostere selected from:

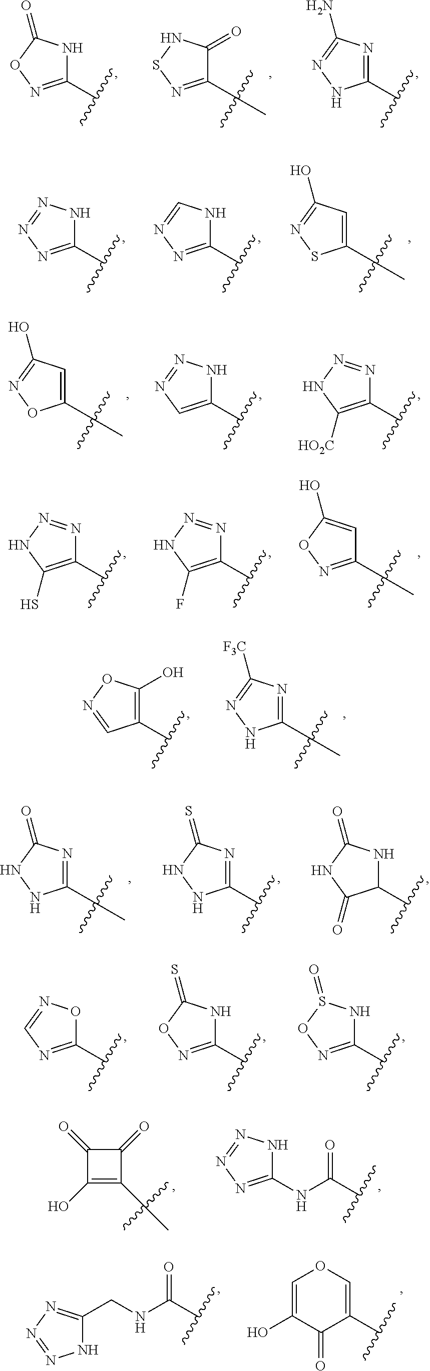

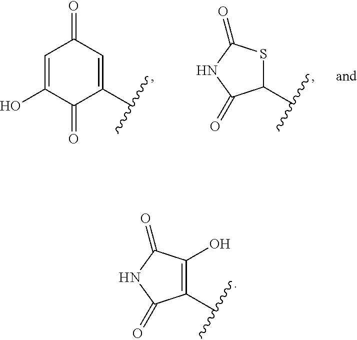 [SMILES: *C1=CC(=O)C=C(O)C1=O ; *C1SC(=O)NC1=O ; *C1=C(O)C(=O)NC1=O]

In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (X) wherein A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —C(R₄)₂B(OR₅)₂, and —CON(R₄)C(R₄)₂B(OR₅)₂; wherein each R₄ is independently H, OH, substituted or unsubstituted alkyl, or substituted or unsubstituted aryl; and R₅ is H or C₁-C₆alkyl.

In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (X) wherein A is a carboxylic acid isostere selected from —SO₃H, —SO₂NHR₄, —P(O)(OR₄)₂, —P(O)(R₄)(OR₄), —C(O)R₄, —CON(R₄)₂, —CONHNHSO₂R₄, —CONHSO₂R₄, —C(R₄)₂B(OR₅)₂, and —CON(R₄)C(R₄)₂B(OR₅)₂; wherein each R₄ is independently H, OH, substituted or unsubstituted alkyl, substituted or unsubstituted heteroaryl, or substituted or unsubstituted aryl; and R₅ is H or C₁-C₆alkyl.

In another embodiment is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (X) wherein R₁ is substituted or unsubstituted alkyl, substituted or unsubstituted heteroalkyl, substituted or unsubstituted heterocycloalkyl, substituted or unsubstituted aryl, or substituted or unsubstituted heteroaryl. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (X) wherein R₁ is H. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (X) wherein R₁ is substituted or unsubstituted alkyl. In further embodiments, R₁ is methyl. In further embodiments, R₁ is ethyl. In further embodiments is a compound of Formula (X) wherein R₁ is propyl. In further embodiments, R₁ is butyl. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (X) wherein R₁ is substituted or unsubstituted heteroalkyl. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (X) wherein R₁ is substituted or unsubstituted heterocycloalkyl. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (X) wherein R₁ is substituted or unsubstituted aryl. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (X) wherein R₁ is substituted or unsubstituted heteroaryl.

In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (X) wherein R₂ is substituted or unsubstituted alkyl, substituted or unsubstituted heteroalkyl, substituted or unsubstituted heterocycloalkyl, substituted or unsubstituted aryl, substituted or unsubstituted heteroaryl, or —CH₂C(O) (substituted or unsubstituted aryl) and R₃ is H. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (X) wherein R₂ is substituted or unsubstituted alkyl, substituted or unsubstituted heteroalkyl, substituted or unsubstituted heterocycloalkyl, substituted or unsubstituted aryl, substituted or unsubstituted heteroaryl, or —CH₂C(O) (substituted or unsubstituted aryl) and R₃ is substituted or unsubstituted alkyl. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (X) wherein R₂ and R₃ together with the carbon atom to which they are attached form a cycloalkyl ring. In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound of Formula (X) wherein R₂ and R₃ together with the carbon atom to which they are attached form a heterocycloalkyl ring.

In some embodiments is a method for treating chemotherapy-induced peripheral neuropathy, or human immunodeficiency virus-associated sensory neuropathy, or symptoms thereof, comprising administering to an individual in need thereof a therapeutically effective amount of a compound having the structure:

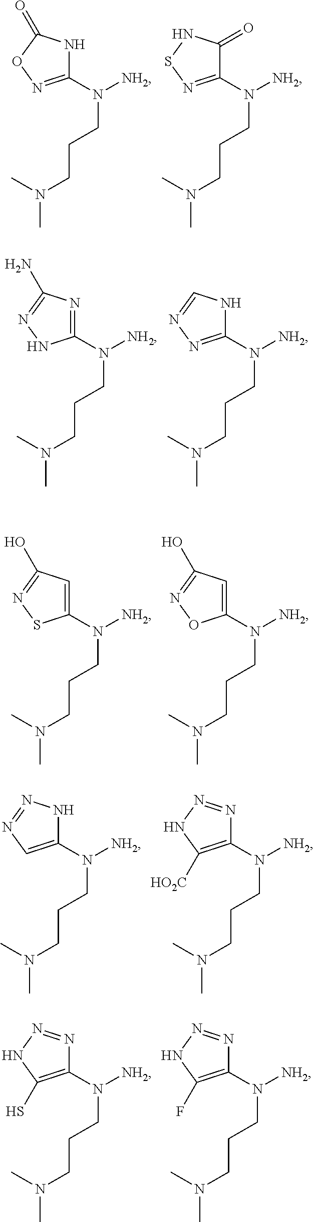

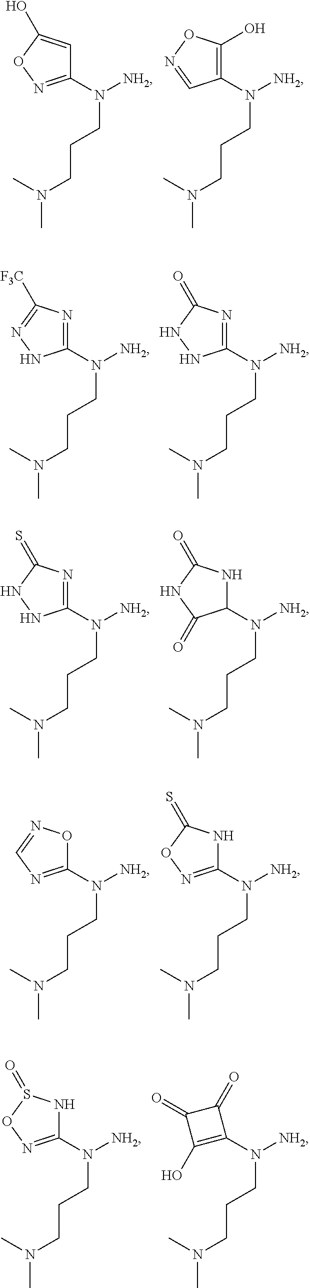

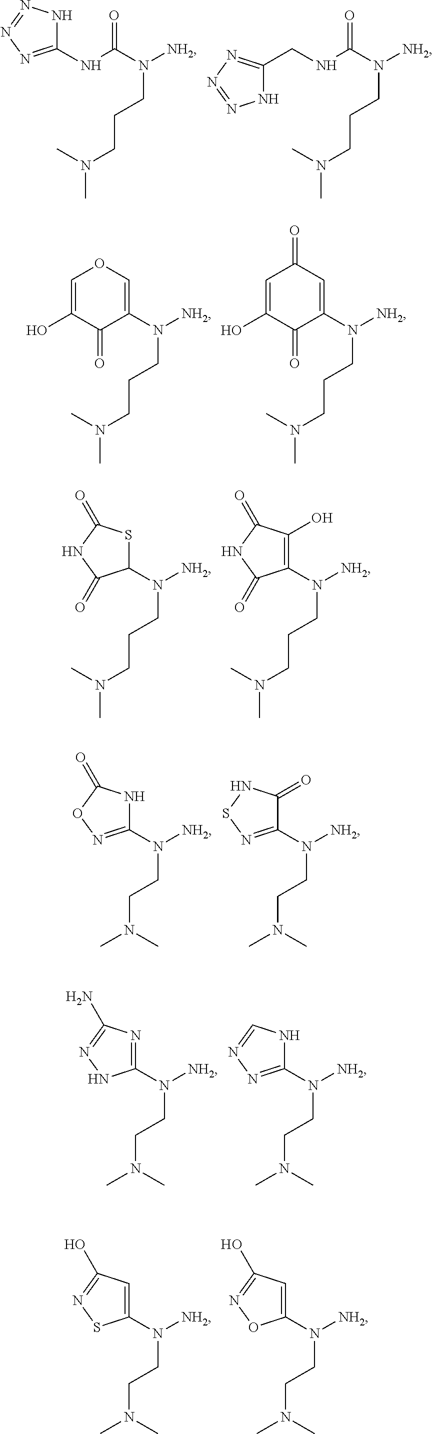

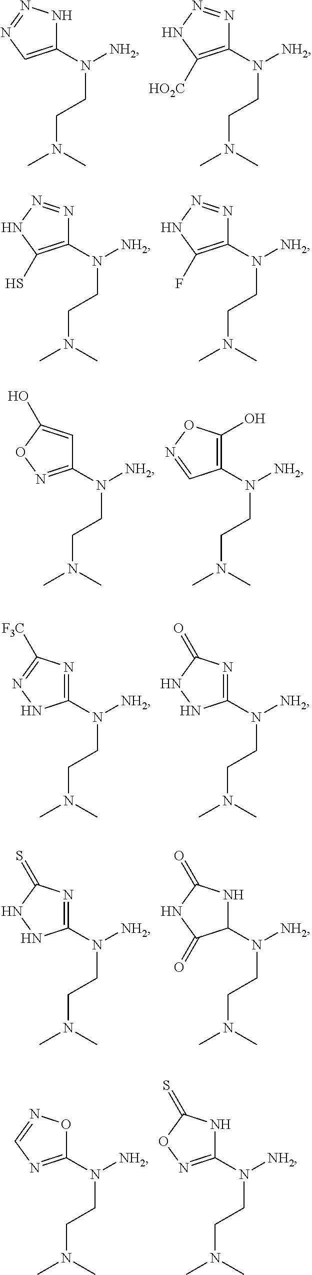

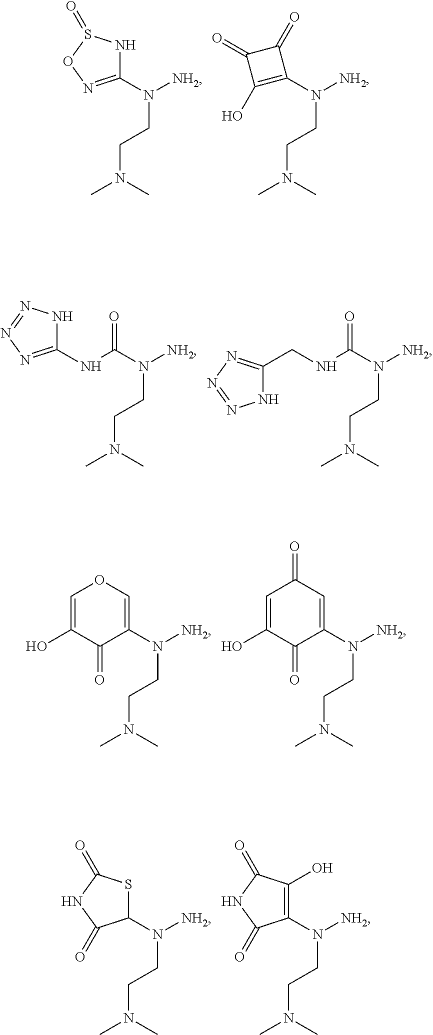

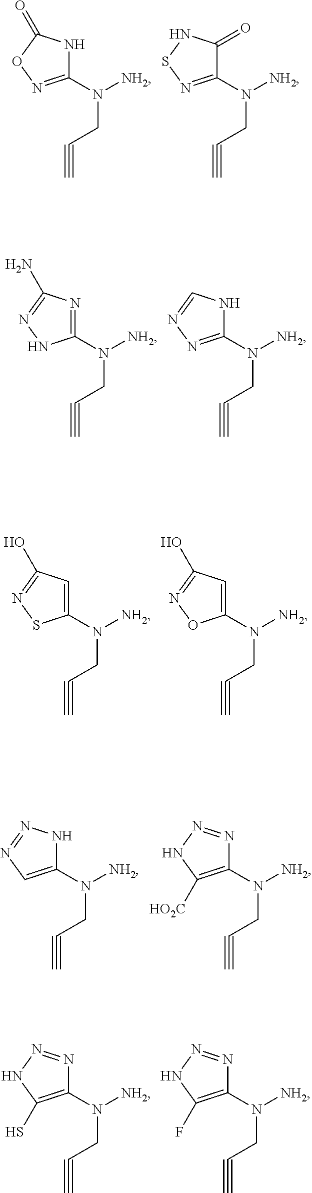

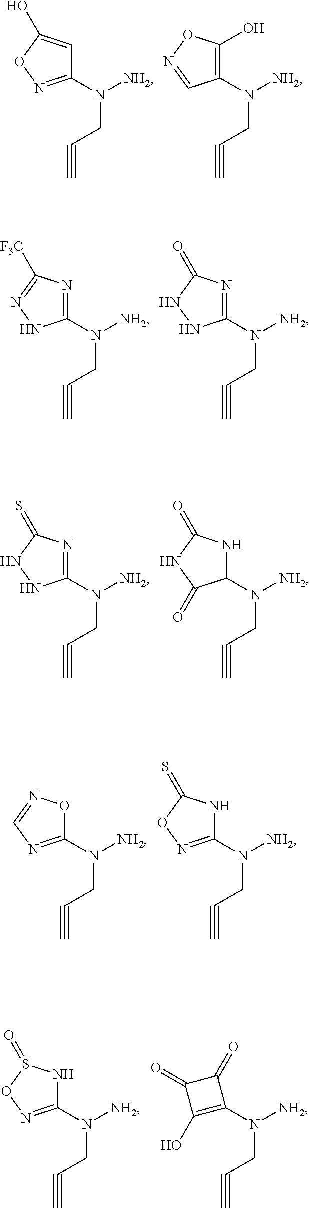

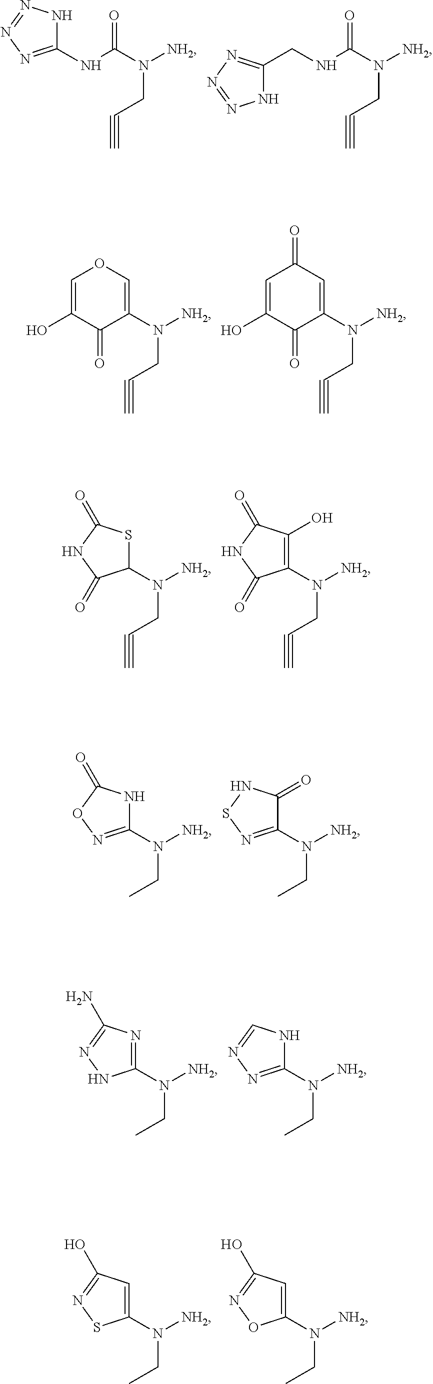

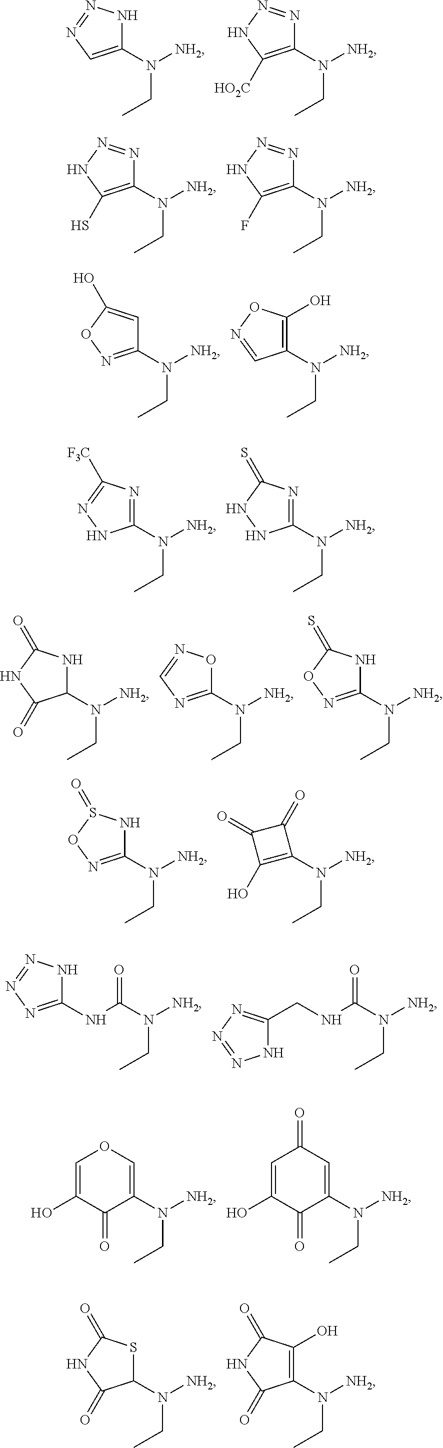

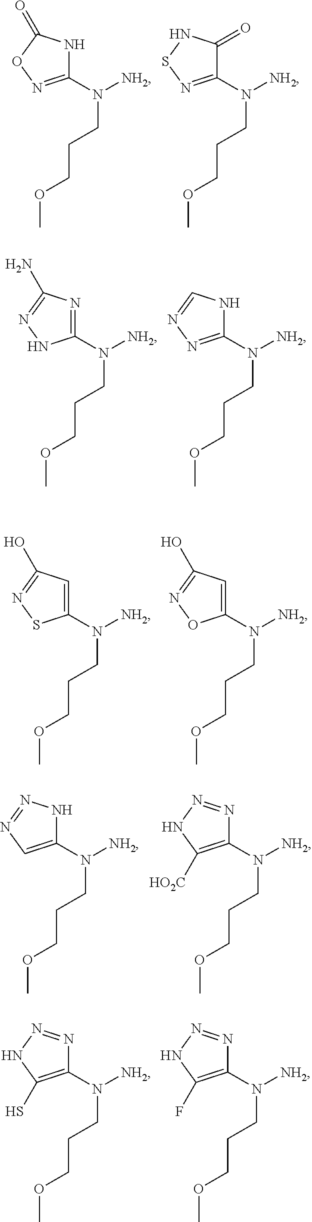

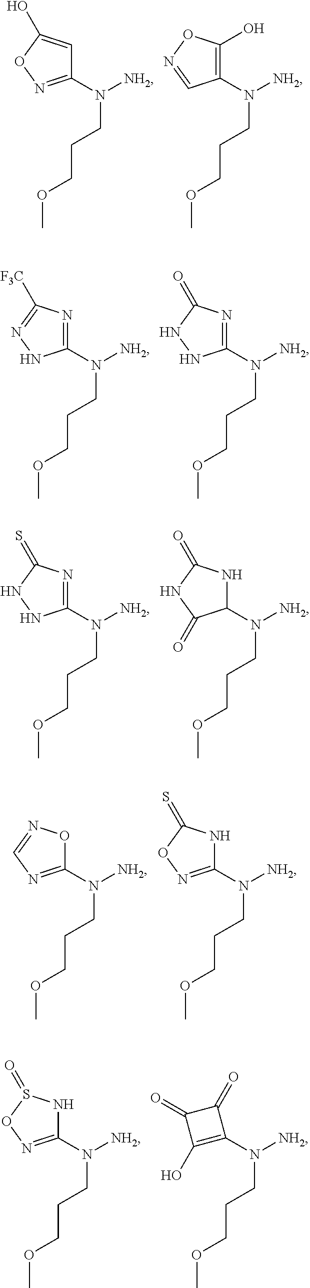

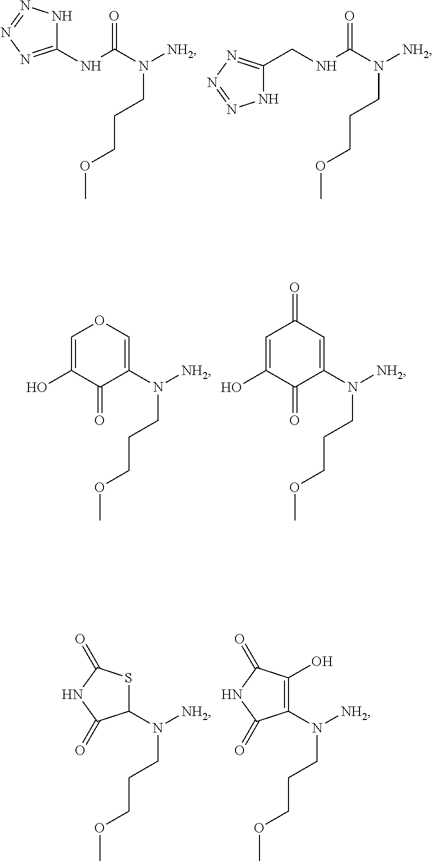 [SMILES: COCCCN(N)C(=O)Nc1nnn[nH]1 ; COCCCN(N)C(=O)NCc1nnn[nH]1 ; COCCCN(N)c1cocc(O)c1=O ; COCCCN(N)C1=CC(=O)C=C(O)C1=O ; COCCCN(N)C1SC(=O)NC1=O ; COCCCN(N)C1=C(O)C(=O)NC1=O]

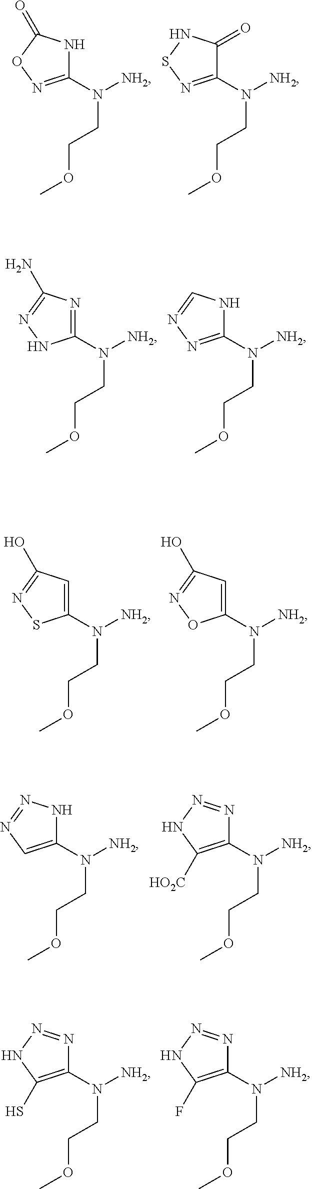

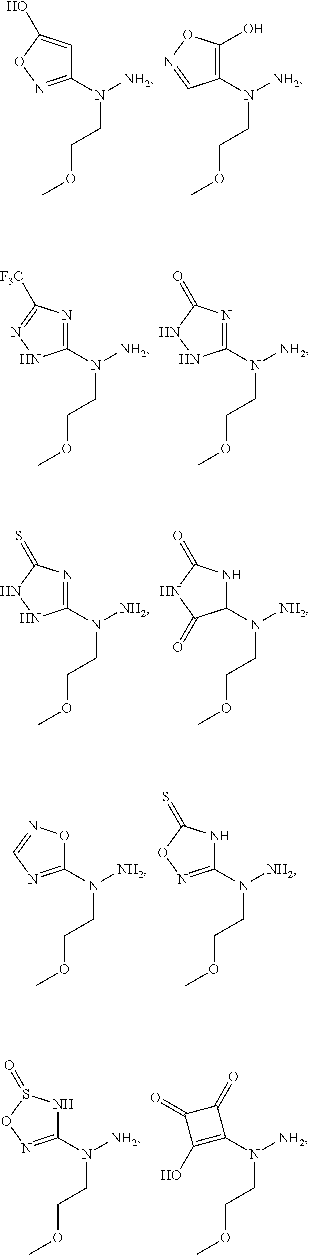

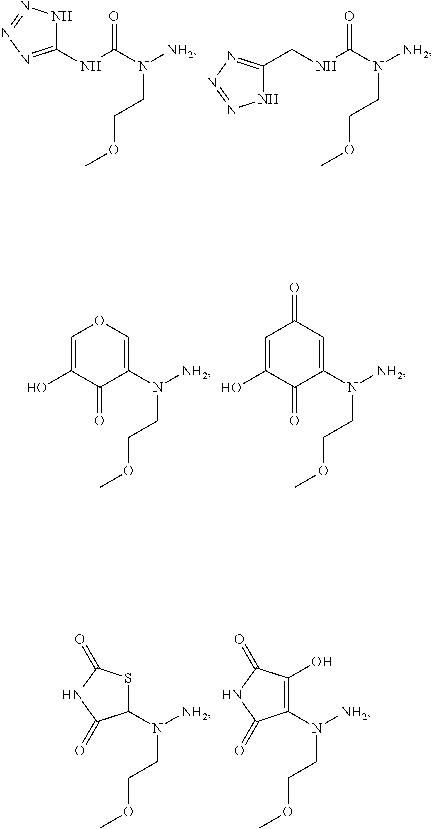

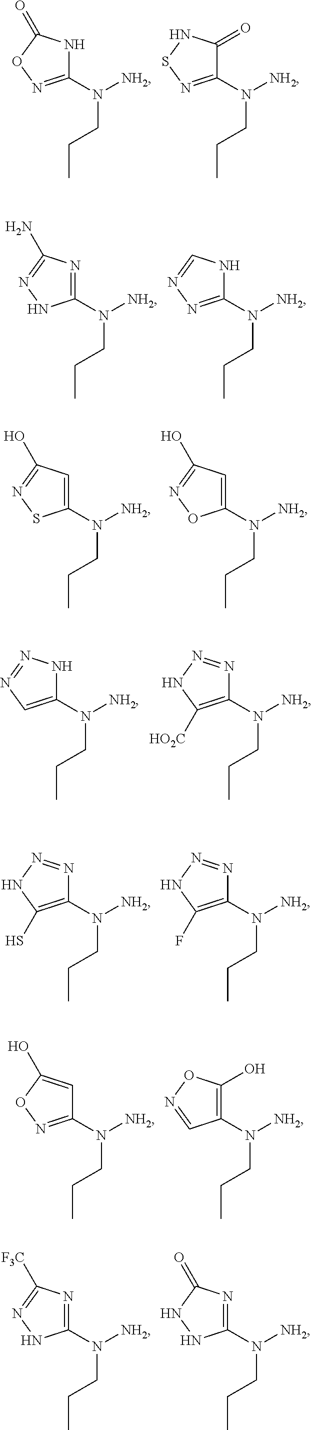

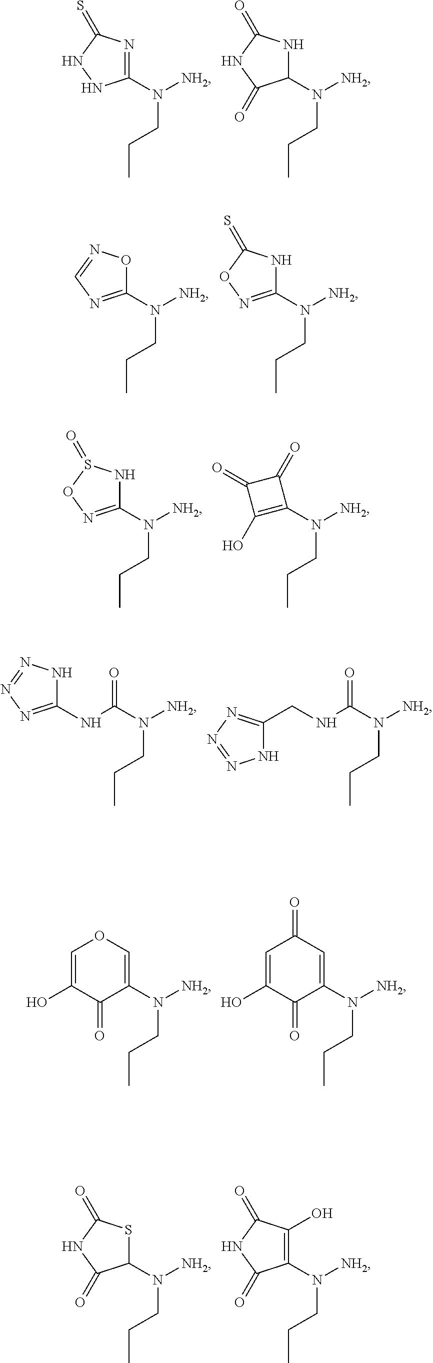

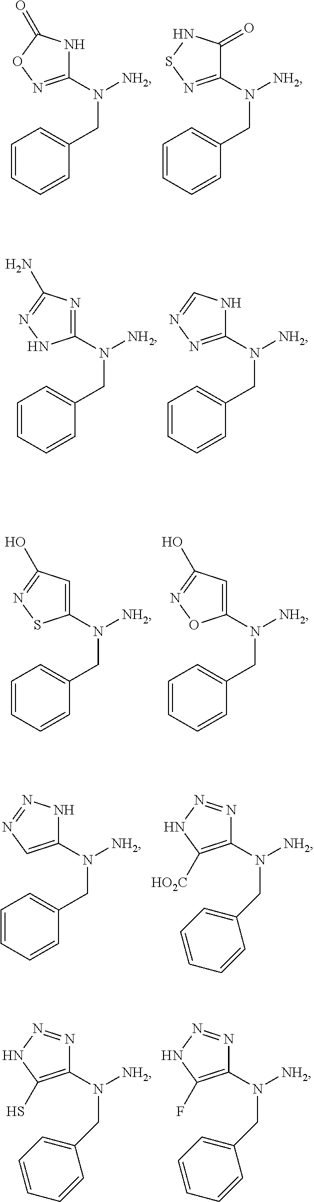

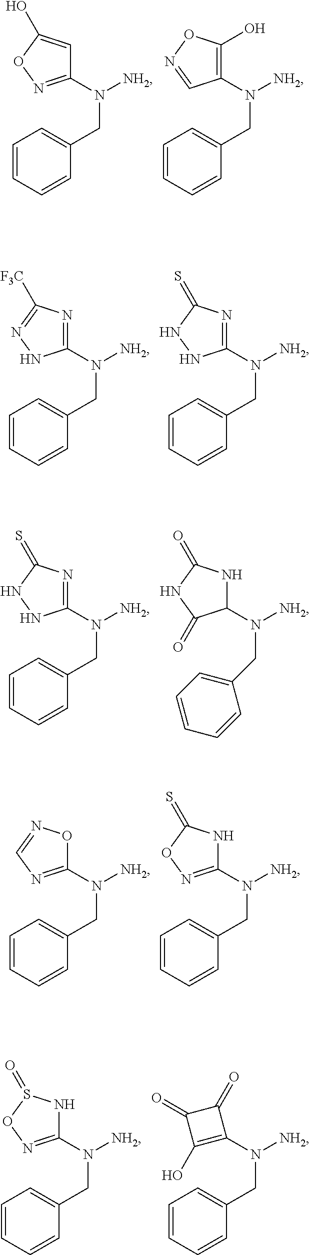

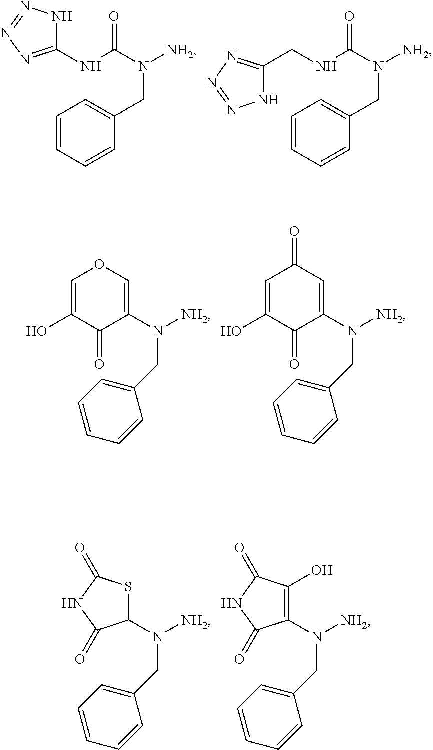 [SMILES: NN(Cc1ccccc1)C(=O)Nc1nnn[nH]1 ; NN(Cc1ccccc1)C(=O)NCc1nnn[nH]1 ; NN(Cc1ccccc1)c1cocc(O)c1=O ; NN(Cc1ccccc1)C1=CC(=O)C=C(O)C1=O ; NN(Cc1ccccc1)C1SC(=O)NC1=O ; NN(Cc1ccccc1)C1=C(O)C(=O)NC1=O]

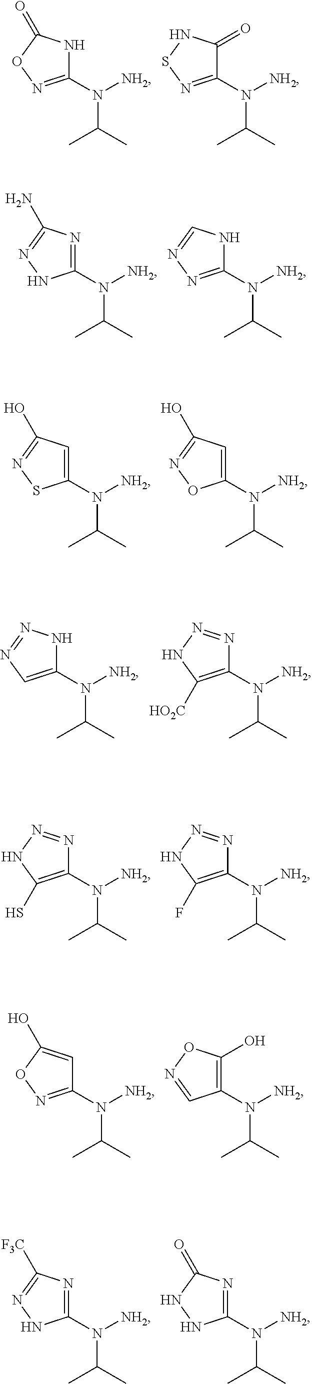

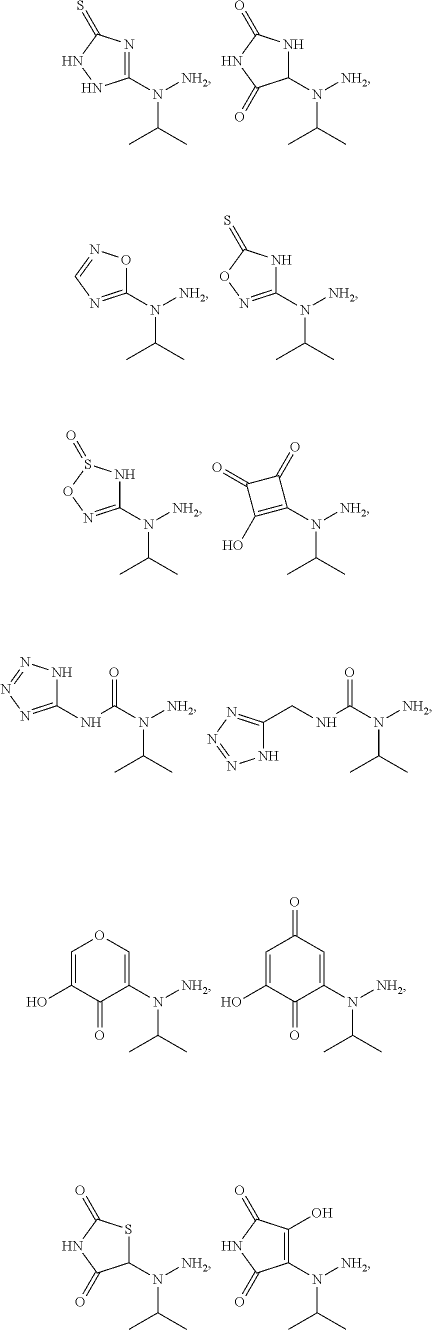

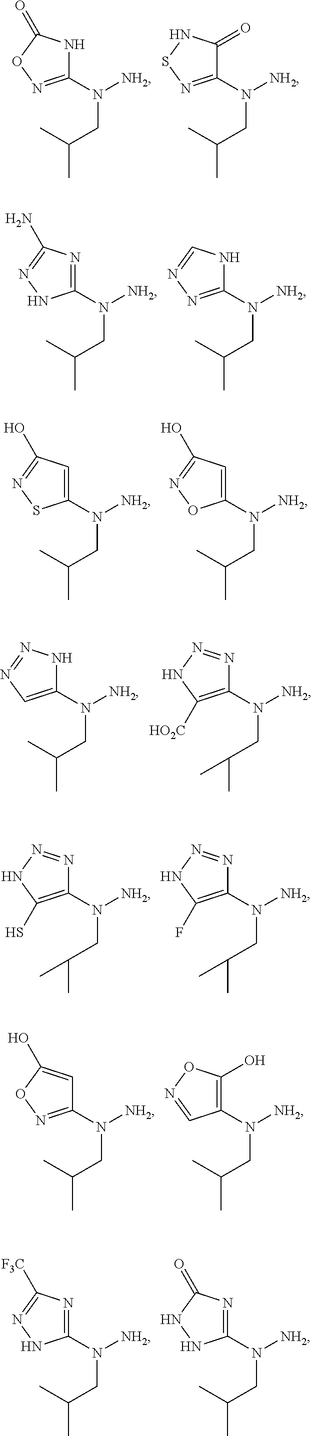

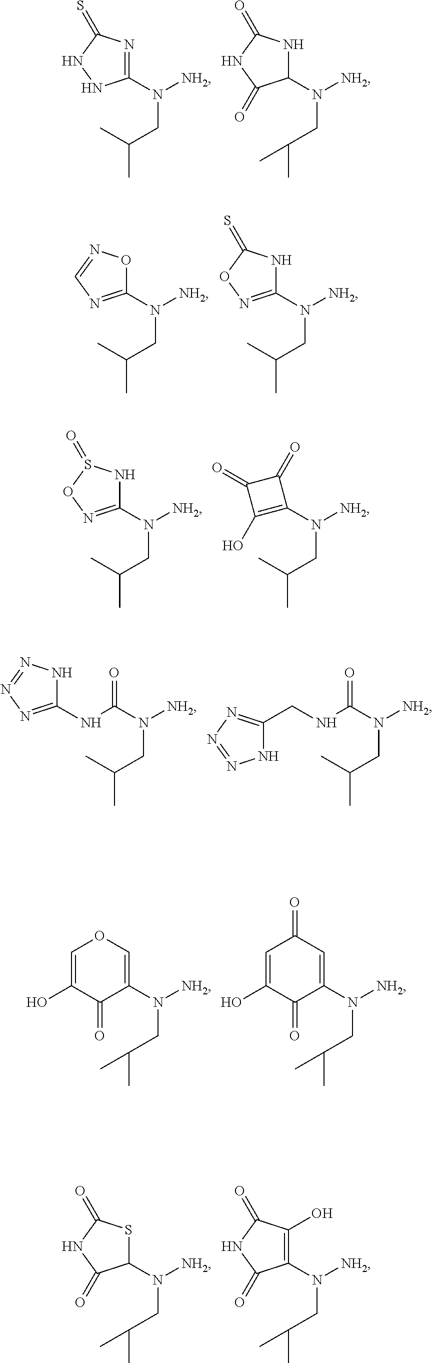

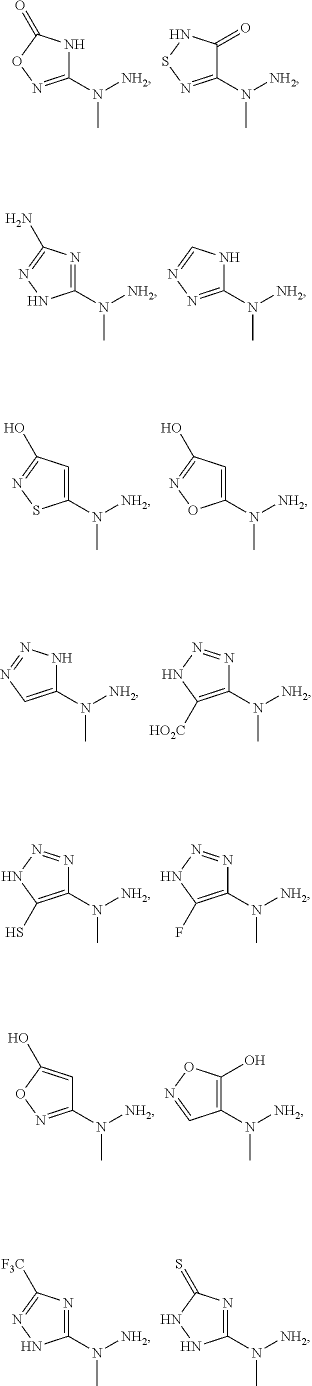

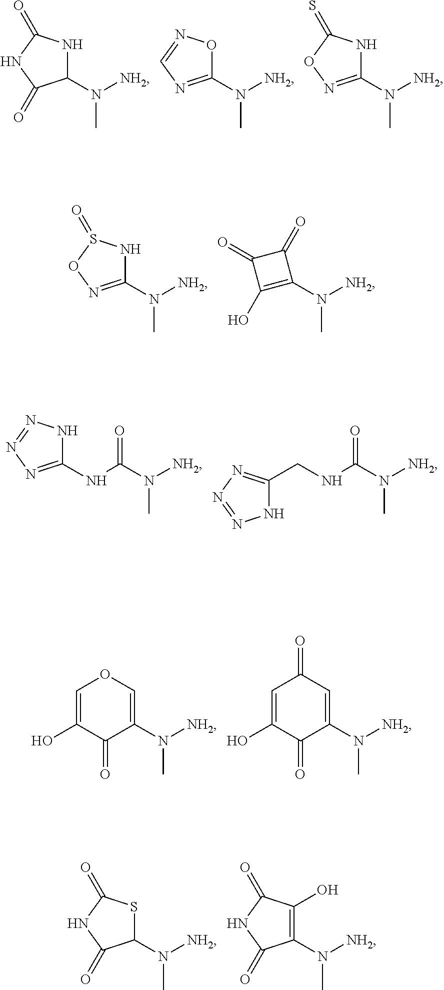

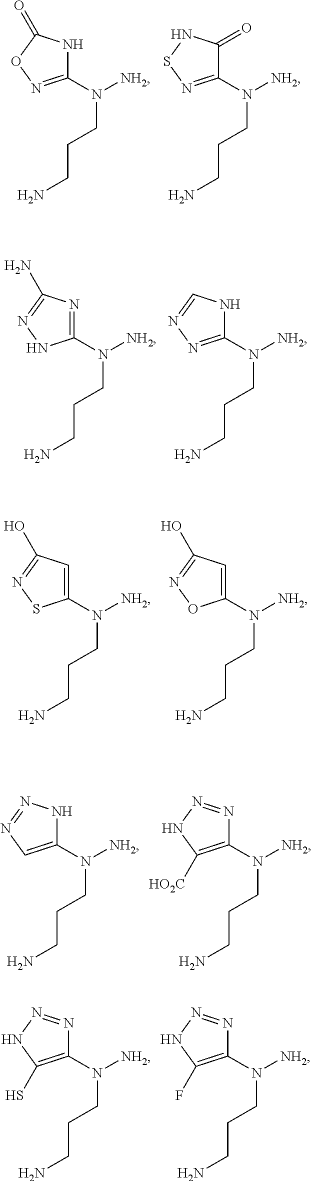

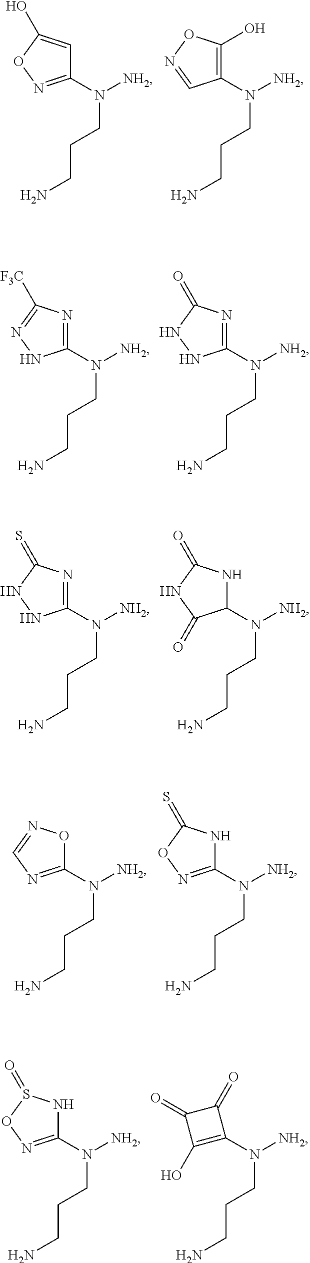

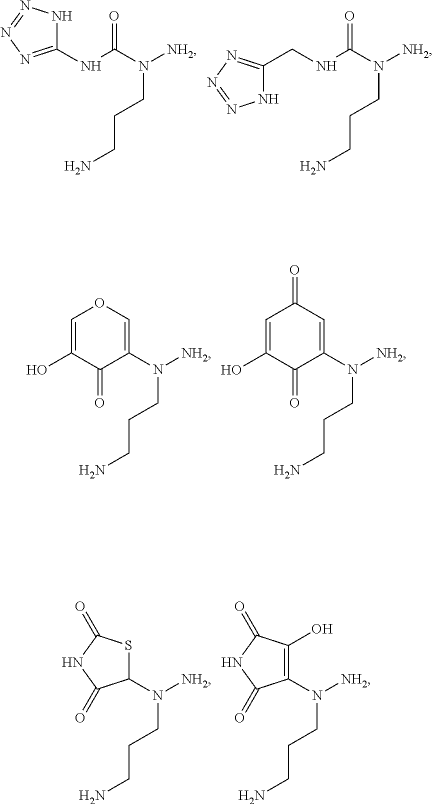 [SMILES: NCCCN(N)C(=O)Nc1nnn[nH]1 ; NCCCN(N)C(=O)NCc1nnn[nH]1 ; NCCCN(N)c1cocc(O)c1=O ; NCCCN(N)C1=CC(=O)C=C(O)C1=O ; NCCCN(N)C1SC(=O)NC1=O ; NCCCN(N)C1=C(O)C(=O)NC1=O]

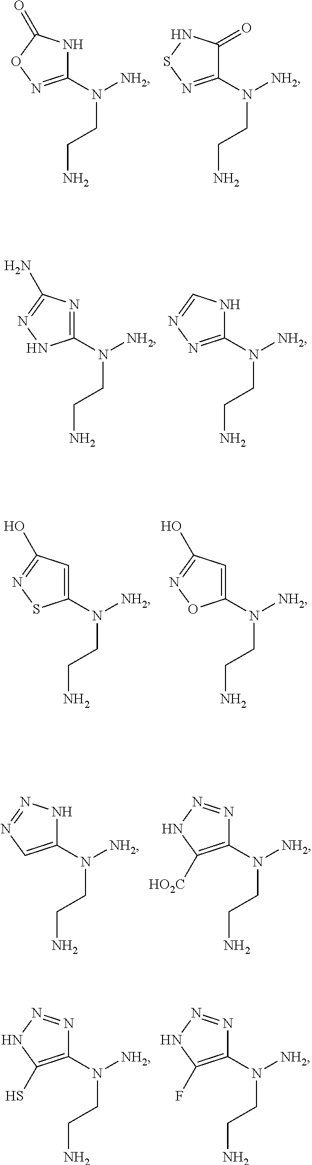

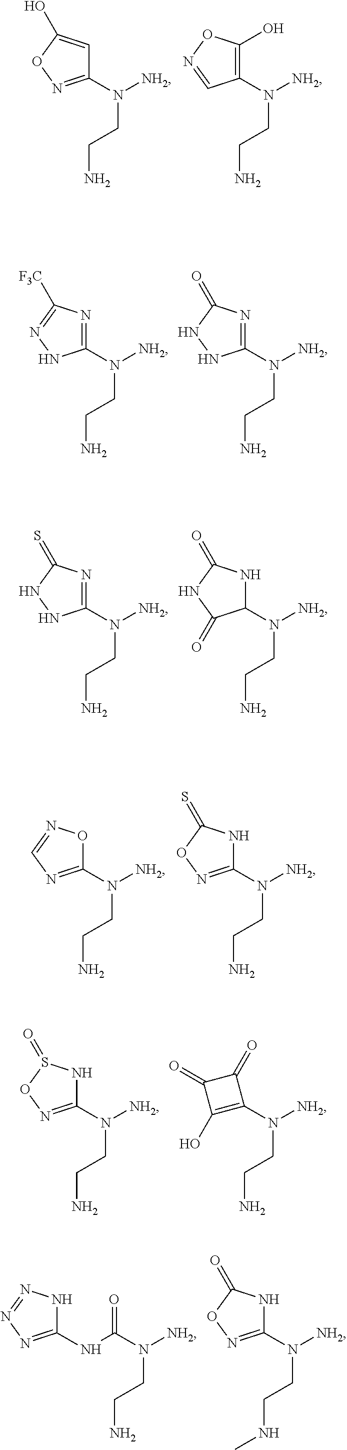

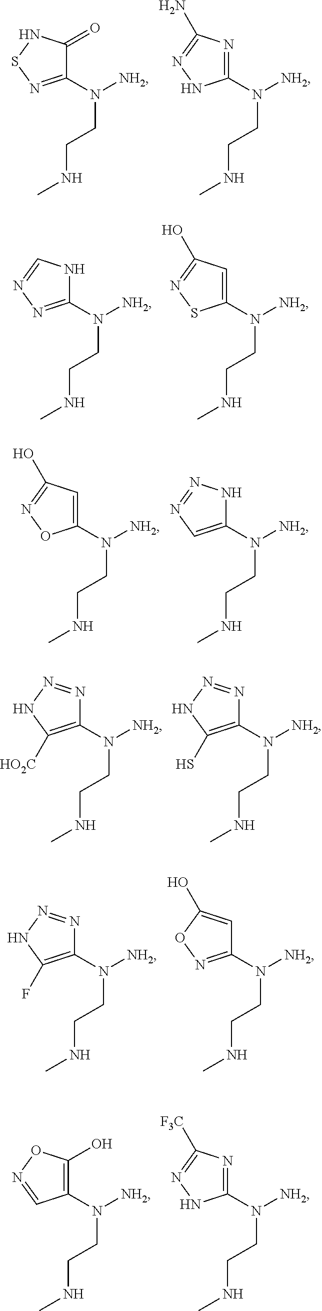

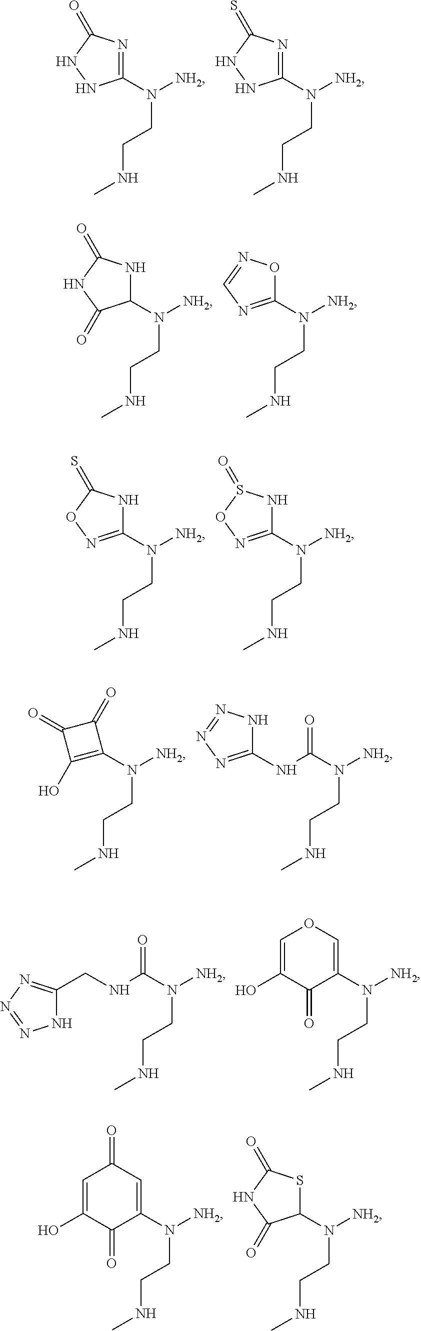

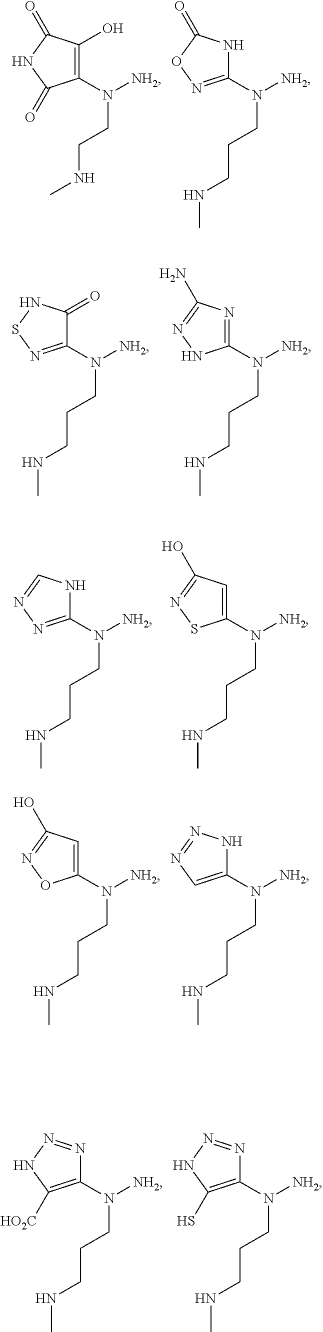

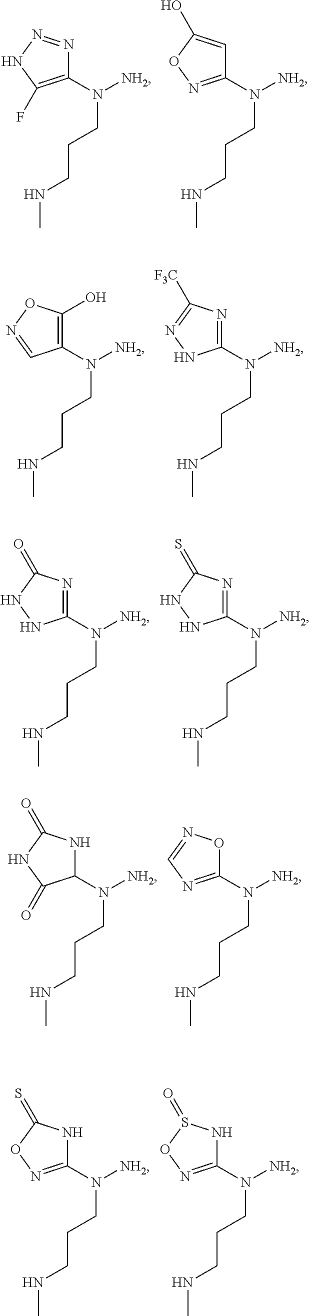

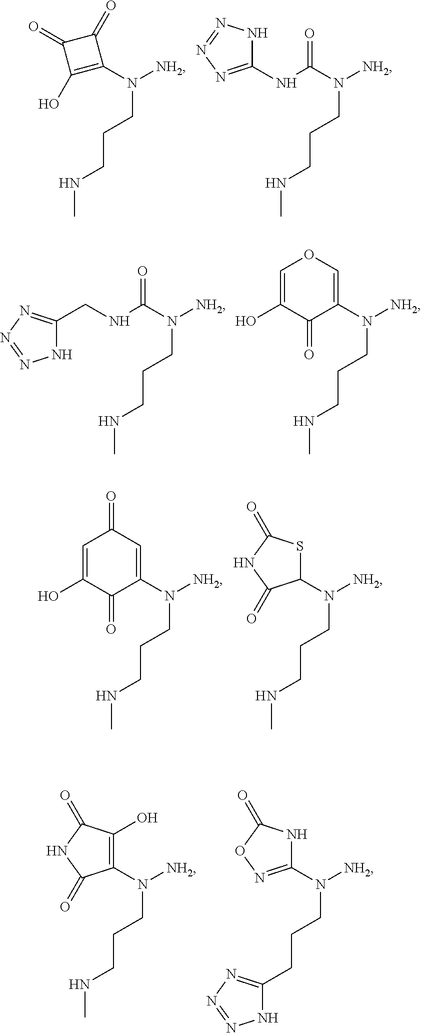

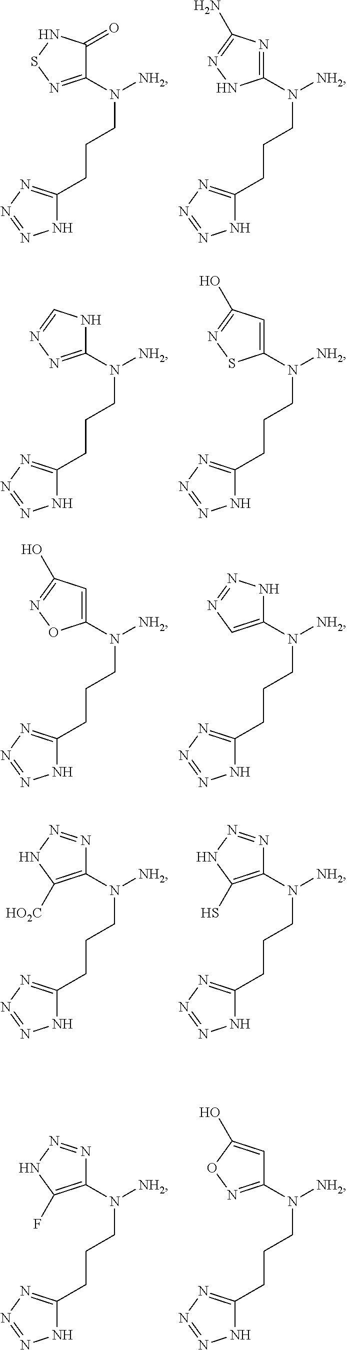

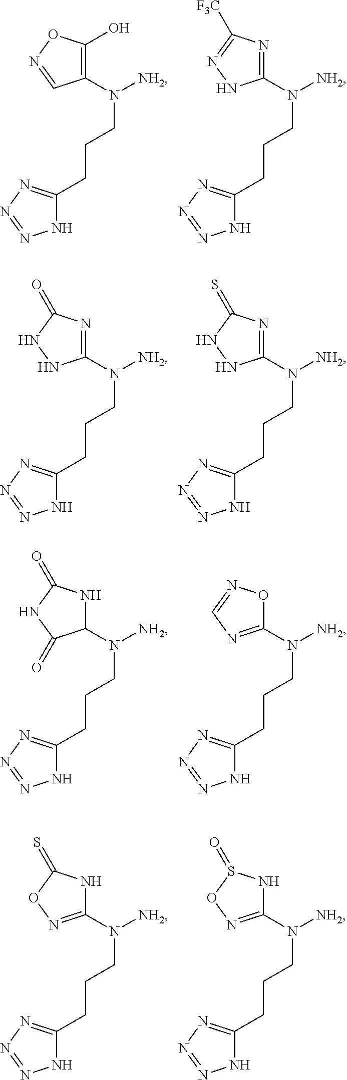

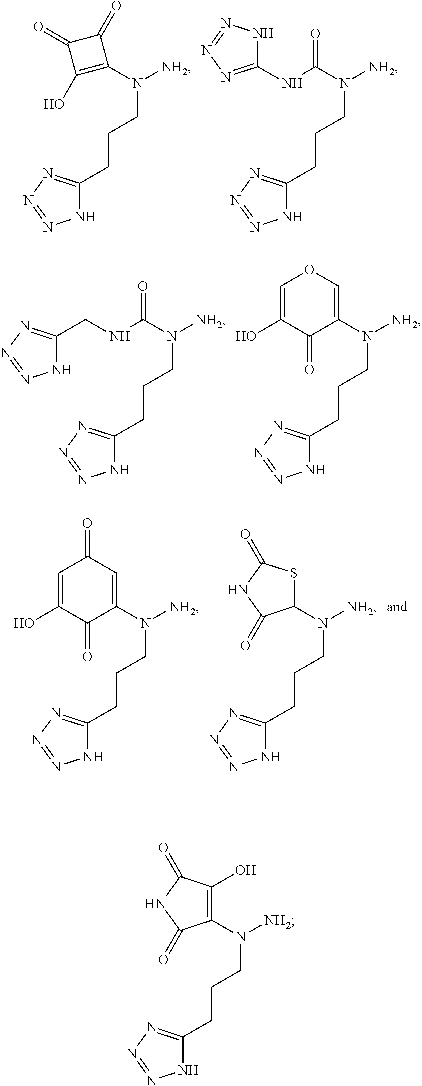

or a pharmaceutically acceptable salt, solvate, or prodrug thereof.

In some embodiments is a method for treating chemotherapy-induced peripheral neuropathy, or human immunodeficiency virus-associated sensory neuropathy, or symptoms thereof, comprising administering to an individual in need thereof a therapeutically effective amount having the structure:

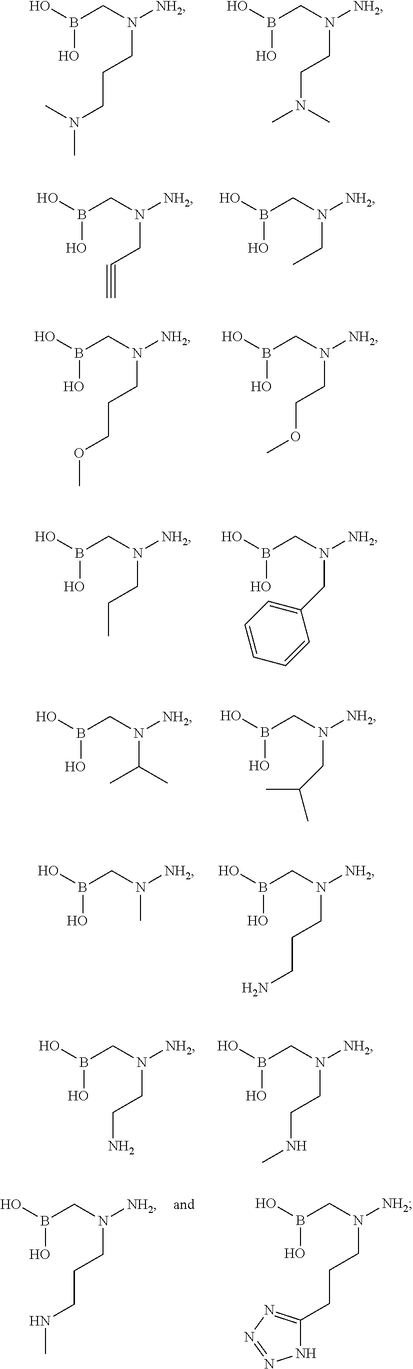

or a pharmaceutically acceptable salt, solvate, or prodrug thereof.

In some embodiments is a method for treating chemotherapy-induced peripheral neuropathy, or symptoms thereof, comprising administering to an individual in need thereof a therapeutically effective amount of a inhibitor of cystathionine-γ-lyase (CSE), wherein the CSE inhibitor is L-propargylglycine, 2-aminopent-4-ynoic acid, (S)-2-aminopent-4-ynoic acid, 2-amino-3-cyanopropanoic acid, (S)-2-amino-3-cyanopropanoic acid, 2-hydrazinylacetic acid hydrochloride, 2-(2-(propan-2-ylidene)hydrazinyl)acetic acid, 4-((2-(1H-tetrazol-5-yl)hydrazinyl)methyl)-N,N-dimethylaniline, (E)-4-((2-(1H-tetrazol-5-yl)hydrazono)methyl)-N,N-diethylaniline, (E)-1-((2-(1H-tetrazol-5-yl)hydrazono)methyl)naphthalen-2-ol, (E)-5-(2-(benzo[d][1,3]dioxol-5-ylmethylene)hydrazinyl)-1H-tetrazole, (E)-4-((2-(1H-tetrazol-5-yl)hydrazono)methyl)phenol, (E)-5-(2-(4-nitrobenzylidene)hydrazinyl)-1H-tetrazole, (E)-5-(2-(furan-2-ylmethylene)hydrazinyl)-1H-tetrazole, 5-hydrazinyl-1H-tetrazole, 5-(1-methylhydrazinyl)-1H-tetrazole, 5-(1-methylhydrazinyl)-1H-1,2,4-triazol-3(2H)-one, 5-(1-ethylhydrazinyl)-1H-1,2,4-triazol-3(2H)-one, or 5-(hydrazinylmethyl)-1H-tetrazole.

In some embodiments is a method for treating human immunodeficiency virus-associated sensory neuropathy, or symptoms thereof, comprising administering to an individual in need thereof a therapeutically effective amount of a inhibitor of cystathionine-γ-lyase (CSE), wherein the CSE inhibitor is L-propargylglycine, 2-aminopent-4-ynoic acid, (S)-2-aminopent-4-ynoic acid, 2-amino-3-cyanopropanoic acid, (S)-2-amino-3-cyanopropanoic acid, 2-hydrazinylacetic acid hydrochloride, 2-(2-(propan-2-ylidene)hydrazinyl)acetic acid, 4-((2-(1H-tetrazol-5-yl)hydrazinyl)methyl)-N,N-dimethylaniline, (E)-4-((2-(1H-tetrazol-5-yl)hydrazono)methyl)-N,N-diethylaniline, (E)-1-((2-(1H-tetrazol-5-yl)hydrazono)methyl)naphthalen-2-ol, (E)-5-(2-(benzo[d][1,3]dioxol-5-ylmethylene)hydrazinyl)-1H-tetrazole, (E)-4-((2-(1H-tetrazol-5-yl)hydrazono)methyl)phenol, (E)-5-(2-(4-nitrobenzylidene)hydrazinyl)-1H-tetrazole, (E)-5-(2-(furan-2-ylmethylene)hydrazinyl)-1H-tetrazole, 5-hydrazinyl-1H-tetrazole, 5-(1-methylhydrazinyl)-1H-tetrazole, 5-(1-methylhydrazinyl)-1H-1,2,4-triazol-3(2H)-one, 5-(1-ethylhydrazinyl)-1H-1,2,4-triazol-3(2H)-one, or 5-(hydrazinylmethyl)-1H-tetrazole.

In some embodiments is a method for treating conditions associated with diabetic neuropathy, or symptoms thereof, comprising administering to an individual in need thereof a therapeutically effective amount of a inhibitor of cystathionine-γ-lyase (CSE), wherein the CSE inhibitor is L-propargylglycine, 2-aminopent-4-ynoic acid, (S)-2-aminopent-4-ynoic acid, 2-amino-3-cyanopropanoic acid, (S)-2-amino-3-cyanopropanoic acid, 2-hydrazinylacetic acid hydrochloride, 2-(2-(propan-2-ylidene)hydrazinyl)acetic acid, 4-((2-(1H-tetrazol-5-yl)hydrazinyl)methyl)-N,N-dimethylaniline, (E)-4-((2-(1H-tetrazol-5-yl)hydrazono)methyl)-N,N-diethylaniline, (E)-1-((2-(1H-tetrazol-5-yl)hydrazono)methyl)naphthalen-2-ol, (E)-5-(2-(benzo[d][1,3]dioxol-5-ylmethylene)hydrazinyl)-1H-tetrazole, (E)-4-((2-(1H-tetrazol-5-yl)hydrazono)methyl)phenol, (E)-5-(2-(4-nitrobenzylidene)hydrazinyl)-1H-tetrazole, (E)-5-(2-(furan-2-ylmethylene)hydrazinyl)-1H-tetrazole, 5-hydrazinyl-1H-tetrazole, 5-(1-methylhydrazinyl)-1H-tetrazole, 5-(1-methylhydrazinyl)-1H-1,2,4-triazol-3(2H)-one, 5-(1-ethylhydrazinyl)-1H-1,2,4-triazol-3(2H)-one, or 5-(hydrazinylmethyl)-1H-tetrazole.

In some embodiments is a method for treating third nerve palsy; diabetic amyotrophy; autonomic neuropathy; or thoracoabdominal neuropathy, or symptoms thereof, comprising administering to an individual in need thereof a therapeutically effective amount of a inhibitor of cystathionine-γ-lyase (CSE), wherein the CSE inhibitor is L-propargylglycine, 2-aminopent-4-ynoic acid, (S)-2-aminopent-4-ynoic acid, 2-amino-3-cyanopropanoic acid, (S)-2-amino-3-cyanopropanoic acid, 2-hydrazinylacetic acid hydrochloride, 2-(2-(propan-2-ylidene)hydrazinyl)acetic acid, 4-((2-(1H-tetrazol-5-yl)hydrazinyl)methyl)-N,N-dimethylaniline, (E)-4-((2-(1H-tetrazol-5-yl)hydrazono)methyl)-N,N-diethylaniline, (E)-1-((2-(1H-tetrazol-5-yl)hydrazono)methyl)naphthalen-2-ol, (E)-5-(2-(benzo[d][1,3]dioxol-5-ylmethylene)hydrazinyl)-1H-tetrazole, (E)-4-((2-(1H-tetrazol-5-yl)hydrazono)methyl)phenol, (E)-5-(2-(4-nitrobenzylidene)hydrazinyl)-1H-tetrazole, (E)-5-(2-(furan-2-ylmethylene)hydrazinyl)-1H-tetrazole, 5-hydrazinyl-1H-tetrazole, 5-(1-methylhydrazinyl)-1H-tetrazole, 5-(1-methylhydrazinyl)-1H-1,2,4-triazol-3(2H)-one, 5-(1-ethylhydrazinyl)-1H-1,2,4-triazol-3(2H)-one, or 5-(hydrazinylmethyl)-1H-tetrazole.

In some embodiments is a method of treating lyme disease, comprising administering to an individual in need thereof a therapeutically effective amount of a inhibitor of cystathionine-γ-lyase (CSE). Also disclosed herein, in certain embodiments, is a method for treating lyme disease comprising administering to an individual in need thereof a therapeutically effective amount of a inhibitor of cystathionine-γ-lyase (CSE), wherein the CSE inhibitor is L-propargylglycine, 2-aminopent-4-ynoic acid, (S)-2-aminopent-4-ynoic acid, 2-amino-3-cyanopropanoic acid, (S)-2-amino-3-cyanopropanoic acid, 2-hydrazinylacetic acid hydrochloride, 2-(2-(propan-2-ylidene)hydrazinyl)acetic acid, 4-((2-(1H-tetrazol-5-yl)hydrazinyl)methyl)-N,N-dimethylaniline, (E)-4-((2-(1H-tetrazol-5-yl)hydrazono)methyl)-N,N-diethylaniline, (E)-1-((2-(1H-tetrazol-5-yl)hydrazono)methyl)naphthalen-2-ol, (E)-5-(2-(benzo[d][1,3]dioxol-5-ylmethylene)hydrazinyl)-1H-tetrazole, (E)-4-((2-(1H-tetrazol-5-yl)hydrazono)methyl)phenol, (E)-5-(2-(4-nitrobenzylidene)hydrazinyl)-1H-tetrazole, (E)-5-(2-(furan-2-ylmethylene)hydrazinyl)-1H-tetrazole, 5-hydrazinyl-1H-tetrazole, 5-(1-methylhydrazinyl)-1H-tetrazole, 5-(1-methylhydrazinyl)-1H-1,2,4-triazol-3(2H)-one, 5-(1-ethylhydrazinyl)-1H-1,2,4-triazol-3(2H)-one, or 5-(hydrazinylmethyl)-1H-tetrazole.

In some embodiments is a method for treating zoster infection comprising administering to an individual in need thereof a therapeutically effective amount of a inhibitor of cystathionine-γ-lyase (CSE), wherein the CSE inhibitor is comprising administering to an individual in need thereof a therapeutically effective amount of a inhibitor of cystathionine-γ-lyase (CSE). Also disclosed herein, in certain embodiments, is a method for treating lyme disease comprising administering to an individual in need thereof a therapeutically effective amount of a inhibitor of cystathionine-γ-lyase (CSE), wherein the CSE inhibitor is L-propargylglycine, 2-aminopent-4-ynoic acid, (S)-2-aminopent-4-ynoic acid, 2-amino-3-cyanopropanoic acid, (S)-2-amino-3-cyanopropanoic acid, 2-hydrazinylacetic acid hydrochloride, 2-(2-(propan-2-ylidene)hydrazinyl)acetic acid, 4-((2-(1H-tetrazol-5-yl)hydrazinyl)methyl)-N,N-dimethylaniline, (E)-4-((2-(1H-tetrazol-5-yl)hydrazono)methyl)-N,N-diethylaniline, (E)-1-((2-(1H-tetrazol-5-yl)hydrazono)methyl)naphthalen-2-ol, (E)-5-(2-(benzo[d][1,3]dioxol-5-ylmethylene)hydrazinyl)-1H-tetrazole, (E)-4-((2-(1H-tetrazol-5-yl)hydrazono)methyl)phenol, (E)-5-(2-(4-nitrobenzylidene)hydrazinyl)-1H-tetrazole, (E)-5-(2-(furan-2-ylmethylene)hydrazinyl)-1H-tetrazole, 5-hydrazinyl-1H-tetrazole, 5-(1-methylhydrazinyl)-1H-tetrazole, 5-(1-methylhydrazinyl)-1H-1,2,4-triazol-3(2H)-one, 5-(1-ethylhydrazinyl)-1H-1,2,4-triazol-3(2H)-one, or 5-(hydrazinylmethyl)-1H-tetrazole.

### Routes of Administration

Suitable routes of administration include, but are not limited to, oral, intravenous, aerosol, parenteral, ophthalmic, pulmonary, transmucosal, transdermal, nasal, and topical administration. In addition, by way of example only, parenteral delivery includes intramuscular, subcutaneous, intravenous, intramedullary injections, as well as intrathecal, direct intraventricular, intraperitoneal, intralymphatic, and/or intranasal injections.

In certain embodiments, a compound of Formula (I), (II), (III), (IV), (V), (VI), (VII), (VIII), (IX), or (X) is administered in a local rather than systemic manner, for example, via topical application of the compound directly on to skin, or intravenously, or subcutaneously, often in a depot preparation or sustained release formulation. In specific embodiments, long acting formulations are administered by implantation (for example subcutaneously or intramuscularly) or by intramuscular injection. In yet other embodiments, the compound as described herein is provided in the form of a rapid release formulation, in the form of an extended release formulation, or in the form of an intermediate release formulation. In yet other embodiments, the compound described herein is administered topically (e.g., as a patch, an ointment, or in combination with a wound dressing, or as a wash or a spray). In alternative embodiments, a formulation is administered systemically (e.g., by injection, or as a pill).

### Pharmaceutical Compositions/Formulations

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), herein incorporated by reference for such disclosure.

Provided herein are pharmaceutical compositions that include a compound of Formula (I), (II), (III), (IV), (V), (VI), (VII), (VIII), (IX), or (X) and at least one pharmaceutically acceptable inactive ingredient. In some embodiments, the compounds described herein are administered as pharmaceutical compositions in which compounds of Formula (I), (II), (III), (IV), (V), (VI), (VII), (VIII), (IX), or (X) are mixed with other active ingredients, as in combination therapy. In other embodiments, the pharmaceutical compositions include other medicinal or pharmaceutical agents, carriers, adjuvants, preserving, stabilizing, wetting or emulsifying agents, solution promoters, salts for regulating the osmotic pressure, and/or buffers. In yet other embodiments, the pharmaceutical compositions include other therapeutically valuable substances.

A pharmaceutical composition, as used herein, refers to a mixture of a compound of Formula (I), (II), (III), (IV), (V), (VI), (VII), (VIII), (IX), or (X) with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

The pharmaceutical formulations described herein are administered to a subject by appropriate administration routes, including but not limited to, oral, parenteral (e.g., intravenous, subcutaneous, intramuscular), intranasal, buccal, topical, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a compound of Formula (I), (II), (III), (IV), (V), (VI), (VII), (VIII), (IX), or (X) are manufactured in a conventional manner, such as, by way of example only, by means of conventional mixing, dissolving, granulating, dragee-making, levigating, emulsifying, encapsulating, entrapping or compression processes.

The pharmaceutical compositions will include at least one compound of Formula (I), (II), (III), (IV), (V), (VI), (VII), (VIII), (IX), or (X) as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, compounds of Formula (I), (II), (III), (IV), (V), (VI), (VII), (VIII), (IX), or (X) exist in unsolvated form or in solvated forms with pharmaceutically acceptable solvents such as water, ethanol, and the like. The solvated forms of the compounds of Formula (I), (II), (III), (IV), (V), (VI), (VII), (VIII), (IX), or (X) are also considered to be disclosed herein.

In some embodiments, the compounds of Formula (I), (II), (III), (IV), (V), (VI), (VII), (VIII), (IX), or (X) exist as tautomers. All tautomers are included within the scope of the compounds presented herein. As such, it is to be understood that a compound of the Formula (I), (II), (III), (IV), (V), (VI), (VII), (VIII), (IX), or (X) or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae drawings within this specification can represent only one of the possible tautomeric forms. However, it is also to be understood that the present disclosure encompasses any tautomeric form, and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been convenient to show graphically herein. For example, tautomerism may be exhibited by a tetrazole group or a triazole group bonded as indicated by the wavy line:

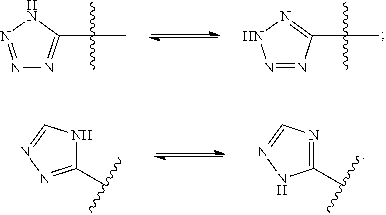 [SMILES: *c1nnn[nH]1 ; *c1nn[nH]n1 ; *c1nnc[nH]1 ; *c1ncn[nH]1]

In some embodiments, compounds of Formula (I), (II), (III), (IV), (V), (VI), (VII), (VIII), (IX), or (X) exist as enantiomers, diastereomers, or other steroisomeric forms. The compounds disclosed herein include all enantiomeric, diastereomeric, and epimeric forms as well as mixtures thereof.

In some embodiments, compounds described herein may be prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrug forms of the herein described compounds, wherein the prodrug is metabolized in vivo to produce a compound of (I), (II), (III), (IV), (V), (VI), (VII), (VIII), (IX), or (X) as set forth herein are included within the scope of the claims. Prodrug forms of the herein described compounds, wherein the prodrug is metabolized in vivo to produce a compound of Formula (I), (II), (III), (IV), (V), (VI), (VII), (VIII), (IX), or (X) as set forth herein are included within the scope of the claims. In some cases, some of the compounds described herein may be a prodrug for another derivative or active compound. In some embodiments described herein, hydrazones are metabolized in vivo to produce a compound of Formula (I), (II), (III), (IV), (V), (VI), (VII), (VIII), (IX), or (X).

In certain embodiments, compositions provided herein include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

The pharmaceutical compositions described herein, which include a compound of Formula (I), (II), (III), (IV), (V), (VI), (VII), (VIII), (IX), or (X) are formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

### Certain Topical Compositions

In some embodiments, compounds of Formula (I), (II), (III), (IV), (V), (VI), (VII), (VIII), (IX), or (X) are prepared as transdermal dosage forms. In one embodiment, the transdermal formulations described herein include at least three components: (1) a formulation of a compound of Formula (I), (II), (III), (IV), (V), (VI), (VII), (VIII), (IX), or (X); (2) a penetration enhancer; and (3) an optional aqueous adjuvant. In some embodiments the transdermal formulations include additional components such as, but not limited to, gelling agents, creams and ointment bases, and the like. In some embodiments, the transdermal formulation is presented as a patch or a wound dressing. In some embodiments, the transdermal formulation further include a woven or non-woven backing material to enhance absorption and prevent the removal of the transdermal formulation from the skin. In other embodiments, the transdermal formulations described herein can maintain a saturated or supersaturated state to promote diffusion into the skin.

In one aspect, formulations suitable for transdermal administration of compounds described herein employ transdermal delivery devices and transdermal delivery patches and can be lipophilic emulsions or buffered, aqueous solutions, dissolved and/or dispersed in a polymer or an adhesive. In one aspect, such patches are constructed for continuous, pulsatile, or on demand delivery of pharmaceutical agents. Still further, transdermal delivery of the compounds described herein can be accomplished by means of iontophoretic patches and the like. In one aspect, transdermal patches provide controlled delivery of a compound of Formula (I), (II), (III), (IV), (V), (VI), (VII), (VIII), (IX), or (X). In one aspect, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In further embodiments, topical formulations include gel formulations (e.g., gel patches which adhere to the skin). In some of such embodiments, a gel composition includes any polymer that forms a gel upon contact with the body (e.g., gel formulations comprising hyaluronic acid, pluronic polymers, poly(lactic-co-glycolic acid (PLGA)-based polymers or the like). In some forms of the compositions, the formulation comprises a low-melting wax such as, but not limited to, a mixture of fatty acid glycerides, optionally in combination with cocoa butter which is first melted. Optionally, the formulations further comprise a moisturizing agent.

In certain embodiments, delivery systems for pharmaceutical compounds may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein may be administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

In alternative embodiments, a compound of Formula (I), (II), (III), (IV), (V), (VI), (VII), (VIII), (IX), or (X) is formulated and presented as a wash or rinse liquid which is used to irrigate the affected area. In further embodiments, a compound of Formula (I), (II), (III), (IV), (V), (VI), (VII), (VIII), (IX), or (X) is formulated and presented as a spray which is applied to the affected area.

### Certain Systemically Administered Compositions

In one aspect, a compound of Formula (I), (II), (III), (IV), (V), (VI), (VII), (VIII), (IX), or (X) is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. In some cases it is desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections or drips or infusions, compounds described herein are formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a compound of Formula (I), (II), (III), (IV), (V), (VI), (VII), (VIII), (IX), or (X) is formulated for use as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

Representative intranasal formulations are described in, for example, U.S. Pat. Nos. 4,476,116, 5,116,817 and 6,391,452. Formulations that include a compound of Formula (I) are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

In some embodiments, pharmaceutical formulations of a compound of Formula (I), (II), (III), (IV), (V), (VI), (VII), (VIII), (IX), or (X) are in the form of a capsules, including push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active compounds are dissolved or suspended in suitable liquids, such as fatty oils, liquid paraffin, or liquid polyethylene glycols. In some embodiments, stabilizers are added. A capsule may be prepared, for example, by placing the bulk blend of the formulation of the compound described above, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to eating.

All formulations for oral administration are in dosages suitable for such administration.

In one aspect, solid oral dosage forms are prepared by mixing a compound of Formula (I), (II), (III), (IV), (V), (VI), (VII), (VIII), (IX), or (X) with one or more of the following: antioxidants, flavoring agents, and carrier materials such as binders, suspending agents, disintegration agents, filling agents, surfactants, solubilizers, stabilizers, lubricants, wetting agents, and diluents.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the pharmaceutical formulation is in the form of a powder.

Compressed tablets are solid dosage forms prepared by compacting the bulk blend of the formulations described above. In various embodiments, tablets will include one or more flavoring agents.

In other embodiments, the tablets will include a film surrounding the final compressed tablet. In some embodiments, the film coating can provide a delayed release of the compound of Formula (I), (II), (III), (IV), (V), (VI), (VII), (VIII), (IX), or (X) from the formulation. In other embodiments, the film coating aids in patient compliance (e.g., Opadry® coatings or sugar coating). Film coatings including Opadry® typically range from about 1% to about 3% of the tablet weight.

In some embodiments, solid dosage forms, e.g., tablets, effervescent tablets, and capsules, are prepared by mixing particles of a compound with one or more pharmaceutical excipients to form a bulk blend composition. The bulk blend is readily subdivided into equally effective unit dosage forms, such as tablets, pills, and capsules. In some embodiments, the individual unit dosages include film coatings. These formulations are manufactured by conventional formulation techniques.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Exemplary useful microencapsulation materials include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® 5100, Eudragit® RD100, Eudragit® E100, Eudragit® L12.5, Eudragit® 512.5, Eudragit® NE30D, and Eudragit® NE 40D, cellulose acetate phthalate, sepifilms such as mixtures of HPMC and stearic acid, cyclodextrins, and mixtures of these materials.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to a CSE inhibitor, the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Buccal formulations that include a compound of Formula (I), (II), (III), (IV), (V), (VI), (VII), (VIII), (IX), or (X) are administered using a variety of formulations known in the art. For example, such formulations include, but are not limited to, U.S. Pat. Nos. 4,229,447, 4,596,795, 4,755,386, and 5,739,136. In addition, the buccal dosage forms described herein can further include a bioerodible (hydrolysable) polymeric carrier that also serves to adhere the dosage form to the buccal mucosa. For buccal or sublingual administration, the compositions may take the form of tablets, lozenges, or gels formulated in a conventional manner.

For intravenous injections, a CSE inhibitor is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, a pharmaceutical composition described herein is in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

Suitable disintegrants for use in the solid dosage forms described herein include, but are not limited to, natural starch such as corn starch or potato starch, a pregelatinized starch, or sodium starch glycolate, a cellulose such as methylcrystalline cellulose, methylcellulose, microcrystalline cellulose, croscarmellose, or a cross-linked cellulose, such as cross-linked sodium carboxymethylcellulose, cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a gum such as agar, guar, locust bean, Karaya, pectin, or tragacanth, sodium starch glycolate, bentonite, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose, hydroxypropylmethylcellulose, hydroxypropylmethylcellulose acetate stearate, hydroxyethylcellulose, hydroxypropylcellulose, ethylcellulose, and microcrystalline cellulose, microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose, glucose, dextrose, molasses, mannitol, sorbitol, xylitol, lactose, a natural or synthetic gum such as acacia, tragacanth, ghatti gum, mucilage of isapol husks, starch, polyvinylpyrrolidone, larch arabogalactan, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Binder levels of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxypropylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms of the pharmaceutical compositions described herein. The amounts of such additives can be readily determined by one skilled in the art, according to the particular properties desired.

In various embodiments, the particles of a compound of Formula (I), (II), (III), (IV), (V), (VI), (VII), (VIII), (IX), or (X) and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In other embodiments, a powder including a compound of Formula (I), (II), (III), (IV), (V), (VI), (VII), (VIII), (IX), or (X) is formulated to include one or more pharmaceutical excipients and flavors. Such a powder is prepared, for example, by mixing the compound and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared. Effervescent salts have been used to disperse medicines in water for oral administration.

### Controlled Release Formulations

In some embodiments, the pharmaceutical dosage forms are formulated to provide a controlled release of a compound of Formula (I), (II), (III), (IV), (V), (VI), (VII), (VIII), (IX), or (X). Controlled release refers to the release of the compound from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein are formulated as enteric coated delayed release oral dosage forms, i.e., as an oral dosage form of a pharmaceutical composition as described herein which utilizes an enteric coating to affect release in the small intestine or large intestine. In one aspect, the enteric coated dosage form is a compressed or molded or extruded tablet/mold (coated or uncoated) containing granules, powder, pellets, beads or particles of the active ingredient and/or other composition components, which are themselves coated or uncoated. In one aspect, the enteric coated oral dosage form is in the form of a capsule containing pellets, beads or granules, which include a compound of Formula (I), (II), (III), (IV), (V), (VI), (VII), (VIII), (IX), or (X), that are coated or uncoated.

Any coatings should be applied to a sufficient thickness such that the entire coating does not dissolve in the gastrointestinal fluids at pH below about 5, but does dissolve at pH about 5 and above. Coatings are typically selected from any of the following:

Shellac—this coating dissolves in media of pH >7; Acrylic polymers—examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine; Poly Vinyl Acetate Phthalate (PVAP)-PVAP dissolves in pH >5, and it is much less permeable to water vapor and gastric fluids.

Conventional coating techniques such as spray or pan coating are employed to apply coatings. The coating thickness must be sufficient to ensure that the oral dosage form remains intact until the desired site of topical delivery in the intestinal tract is reached.

In other embodiments, the formulations described herein are delivered using a pulsatile dosage form. A pulsatile dosage form is capable of providing one or more immediate release pulses at predetermined time points after a controlled lag time or at specific sites. Exemplary pulsatile dosage forms and methods of their manufacture are disclosed in U.S. Pat. Nos. 5,011,692, 5,017,381, 5,229,135, 5,840,329 and 5,837,284. In one embodiment, the pulsatile dosage form includes at least two groups of particles, (i.e. multiparticulate) each containing the formulation described herein. The first group of particles provides a substantially immediate dose of the compound of Formula (I) upon ingestion by a mammal. The first group of particles can be either uncoated or include a coating and/or sealant. In one aspect, the second group of particles comprises coated particles. The coating on the second group of particles provides a delay of from about 2 hours to about 7 hours following ingestion before release of the second dose. Suitable coatings for pharmaceutical compositions are described herein or known in the art.

In some embodiments, pharmaceutical formulations are provided that include particles of a compound of Formula (I), (II), (III), (IV), (V), (VI), (VII), (VIII), (IX), or (X) and at least one dispersing agent or suspending agent for oral administration to a subject. The formulations may be a powder and/or granules for suspension, and upon admixture with water, a substantially uniform suspension is obtained.

In some embodiments, particles formulated for controlled release are incorporated in a gel or a patch or a wound dressing.

In one aspect, liquid formulation dosage forms for oral administration and/or for topical administration as a wash are in the form of aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the particles of a compound of Formula (I), (II), (III), (IV), (V), (VI), (VII), (VIII), (IX), or (X), the liquid dosage forms include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

In some embodiments, the liquid formulations also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state, as defined in The USP Pharmacists' Pharmacopeia (2005 edition, chapter 905), for at least 4 hours. In one embodiment, an aqueous suspension is re-suspended into a homogenous suspension by physical agitation lasting less than 1 minute. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch, or sodium starch glycolate; a cellulose such as methylcrystalline cellulose, methylcellulose, croscarmellose, or a cross-linked cellulose, such as cross-linked sodium carboxymethylcellulose, cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein include, for example, hydrophilic polymers, electrolytes, Tween® 60 or 80, PEG, polyvinylpyrrolidone, and the carbohydrate-based dispersing agents such as, for example, hydroxypropylcellulose and hydroxypropyl cellulose ethers, hydroxypropyl methylcellulose and hydroxypropyl methylcellulose ethers, carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer, 4-(1,1,3,3-tetramethylbutyl)-phenol polymer with ethylene oxide and formaldehyde (also known as tyloxapol), poloxamers; and poloxamines. In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers; hydroxypropyl methylcellulose and hydroxypropyl methylcellulose ethers; carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers; or poloxamines.

Wetting agents suitable for the aqueous suspensions and dispersions described herein include, but are not limited to, cetyl alcohol, glycerol monostearate, polyoxyethylene sorbitan fatty acid esters (e.g., the commercially available Tweens® such as e.g., Tween 20® and Tween 80®, and polyethylene glycols, oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, aspartame, chocolate, cinnamon, citrus, cocoa, cyclamate, dextrose, fructose, ginger, glycyrrhetinate, glycyrrhiza (licorice) syrup, monoammonium glyrrhizinate (MagnaSweet), maltol, mannitol, menthol, neohesperidine DC, neotame, Prosweet® Powder, saccharin, sorbitol, stevia, sucralose, sucrose, sodium saccharin, saccharin, aspartame, acesulfame potassium, mannitol, sucralose, tagatose, thaumatin, vanilla, xylitol, or any combination thereof.

### Methods of Dosing and Treatment Regimens

A method for treating any of the diseases or conditions described herein in a subject in need of such treatment, involves administration of pharmaceutical compositions that include at least one compound of Formula (I), (II), (III), (IV), (V), (VI), (VII), (VIII), (IX), or (X) or a pharmaceutically acceptable salt, pharmaceutically acceptable prodrug, or pharmaceutically acceptable solvate thereof, in therapeutically effective amounts to said subject. In another embodiment, the compounds of Formula (I), (II), (III), (IV), (V), (VI), (VII), (VIII), (IX), or (X) are used in the preparation of medicaments for treating chemotherapy-induced peripheral neuropathy, or human immunodeficiency virus-associated sensory neuropathy, or symptoms thereof. In some embodiments, the compounds of Formula (I), (II), (III), (IV), (V), (VI), (VII), (VIII), (IX), or (X) are used in the preparation of medicaments for treating chemotherapy-induced peripheral neuropathy, or symptoms thereof. In some embodiments, the compounds of Formula (I), (II), (III), (IV), (V), (VI), (VII), (VIII), (IX), or (X) are used in the preparation of medicaments for treating human immunodeficiency virus-associated sensory neuropathy, or symptoms thereof.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a compound of Formula (I), (II), (III), (IV), (V), (VI), (VII), (VIII), (IX), or (X) in order to prevent a return of the symptoms of the disease or condition.

In certain embodiments wherein the patient's condition does not improve, upon the doctor's discretion the administration of the compound of Formula (I), (II), (III), (IV), (V), (VI), (VII), (VIII), (IX), or (X) is administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered may be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

In certain embodiments the dose of drug being administered may be temporarily reduced or temporarily suspended for a certain length of time (i.e., a “drug diversion”). In specific embodiments, the length of the drug diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some embodiments, once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but can nevertheless be determined according to the particular circumstances surrounding the case, including, e.g., the specific agent being administered, the route of administration, the condition being treated, and the subject or host being treated. In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously (or over a short period of time) or at appropriate intervals, for example as two, three, four or more sub-doses per day.

In some embodiments, as a patient is started on a regimen of a CSE inhibitor, the patient is also weaned off (e.g., step-wise decrease in dose) a second treatment regimen (e.g., a methylxanthine).

In one embodiment, the daily dosages appropriate for a compound of Formula (I), (II), (III), (IV), (V), (VI), (VII), (VIII), (IX), or (X) described herein are from about 0.01 to about 10 mg/kg per body weight. In specific embodiments, an indicated daily dosage in a large mammal, including, but not limited to, humans, is in the range from about 0.5 mg to about 1000 mg, conveniently administered in divided doses, including, but not limited to, up to four times a day. In one embodiment, the daily dosage is administered in extended release form. In certain embodiments, suitable unit dosage forms for oral administration comprise from about 1 to 500 mg active ingredient. In other embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

### Combination Therapy

In one embodiment, the CSE inhibitors of Formula (I), (II), (III), (IV), (V), (VI), (VII), (VIII), (IX), or (X) are administered to an individual in need thereof in combination with an anti-inflammatory agent. Examples of such anti-inflammatory agents include and are not limited to analgesics, non-steroidal anti-inflammatory drugs (NSAIDs), COX-2 inhibitors, and the like.

In another embodiment, the CSE inhibitors of Formula (I), (II), (III), (IV), (V), (VI), (VII), (VIII), (IX), or (X) are administered to an individual in need thereof in combination with a pain medication. Examples of such pain medications include and are not limited to paracetamol, gabapentin, pregablin, duloxetine, the non-steroidal anti-inflammatory drugs (NSAIDs) such as the salicylates, opioid drugs such as morphine and opium, and analogues such as codeine, oxycodone and the like, as well as opioid-sparing compounds.

In further embodiments, the CSE inhibitors of Formula (I), (II), (III), (IV), (V), (VI), (VII), (VIII), (IX), or (X) are administered to an individual in need thereof in combination with an anesthetic agent (e.g., benzocaine, lidocaine and the like).

### Combination Formulations and Kits

Also provided herein are kits for therapies described herein. In some embodiments, the kit comprises a CSE inhibitor and a second treatment regimen. Such kits generally will comprise one or more of the active agent as disclosed herein, and instructions for using the kit.

In some embodiments, kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In other embodiments, the containers are formed from a variety of materials such as glass or plastic.

In certain embodiments, the pharmaceutical compositions are presented in a pack or dispenser device which contains one or more unit dosage forms containing a CSE inhibitor. In another embodiment, the pack for example contains metal or plastic foil, such as a blister pack.

### Assays for Identification of CSE Inhibitors

In some embodiments, CSE inhibitors are identified by use of in vitro assays. By way of example, an in vitro assay for CSE enzyme activity is described in Zhong et al. Chinese Medical Journal, 2009, 122, 326-330. In some embodiments, in vitro enzyme assays are adapted for high-throughput screening (HTS) using any suitable method.

In some embodiments, in vivo assays are used to determine the effect of CSE inhibitor. In some embodiments, an in vivo assay for identifying a CSE inhibitor comprises:

(a) preparing organ or tissue homogenates from a test animal that has been administered a test compound; and

(b) calculating H₂S concentration based on absorbance;

wherein a decrease in H₂S concentration indicates that the test compound is a CSE inhibitor. In some embodiments of the aforementioned assay, the test animal is subjected to normoxia, acute hypoxia, chronic intermittent hypoxia, hypercapnia, or a combination thereof. Optional intermediate steps include:

effecting enzymatic reaction on L-cysteine;

quenching the enzymatic reaction with zinc acetate and trichloroacetic acid;

reacting the zinc sulfide with acidic N,N-dimethyl-p-phenylendiamine sulfate and ferric chloride; and

measuring the absorbance of the assay mixture with a micro-plate reader.

### EXAMPLES

Measurements of H₂S Levels.

H₂S levels in the liver were assayed as follows. Briefly, liver tissue homogenates were prepared in 100 mM potassium phosphate buffer, pH 7.4+0.5% Triton-X100. The enzyme reaction was carried out in 96 well, deep square well plates with 700 μl Glass Insert (Waters Corporation Cat. #186000349) with TFE/Silicone MicroMat sealing covers (Sun-SRI Cat. #400 026). In the outer well in a total volume of 200 μl the assay mixture contained (in final concentration): L-cysteine, (5 mM); pyridoxal 5′-phosphate, (50 μM); potassium phosphate buffer, pH 7.4, (100 mM); and tissue homogenate (500 μg protein). The glass insert contained 100 μl alkaline zinc acetate solution (1% in 0.1N NaOH) to trap the generated H₂S. The reaction mixture was incubated at 37° C. for 3 h and at the end of the reaction, 100 μl N,N-dimethyl-p-phenylenediamine sulfate (20 μM in 7N HCl) and 100 μl ferric chloride (30 μM in 1.2N HCl) was added to the glass insert. Absorbance was measured at 671 nm using a micro-plate reader. A standard curve relating the concentration of NaHS and absorbance was used to calculate H₂S concentration and expressed as nanomoles of H₂S formed per hour per milligram protein.

### Example 1

### CSE In Vitro Assay

Test compounds (from DMSO stock solutions) were added to (final concentrations) 20 ug/ml enzyme solution (human, mouse or rat recombinant CSE) plus 50 uM PLP in assay buffer (100 mM potassium phosphate pH 7.6) in 96 well plates in total volume of 190 ul. Plates were incubated for 30 minutes at room temperature before the addition of 10 ul of 200 mM (20× final in assay buffer) DL-Homocysteine substrate to each well. Plates were incubated at 37° C. for 3 hours. 50 ul 20 mM DMPDA in 7.2N HCl was added to each well followed by 50 ul 30 mM FeCl₃ in 1.2N HCl. Plates were incubated for 10 minutes with shaking at room temperature and then absorbance at 671 nm read in Promega GloMax microplate reader.

TABLE 1

| | | IC50 |
|---|---|---|
| Compound | Structure | (μM) |
| 1 | 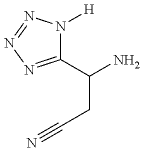 | C |
| 2 | 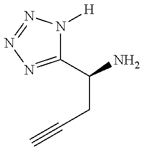 | A |
| 3 | 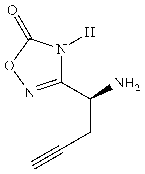 | A |
| 4 | 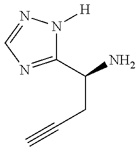 | A |
| 5 | 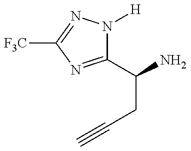 | C |
| 6 | 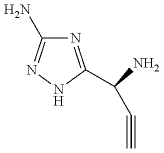 | C |
| 7 | 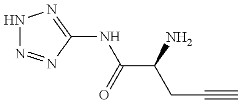 | B |
| 8 | 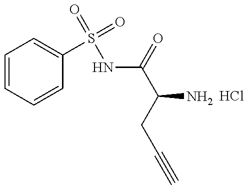 [SMILES: C#CC[C@H](N)C(=O)NS(=O)(=O)c1ccccc1 ; Cl] | B |
| 9 | 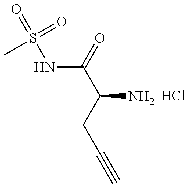 | B |
| 10 | 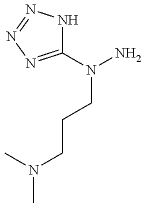 | B |
| 11 | 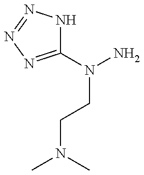 | C |
| 12 | 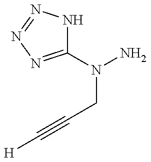 | A |
| 13 | 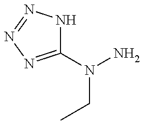 | A |
| 14 | 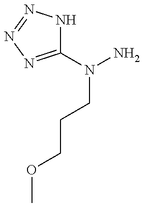 | C |
| 15 | 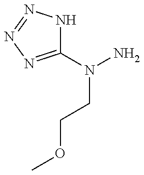 | B |
| 16 | 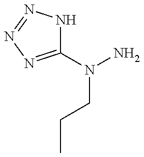 | B |
| 17 | 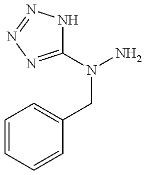 [SMILES: NN(Cc1ccccc1)c1nnn[nH]1] | C |
| 18 | 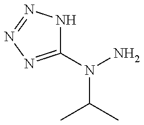 | C |
| 19 | 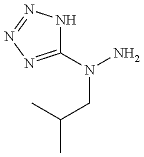 | C |
| 20 | 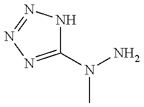 | A |
| 21 | 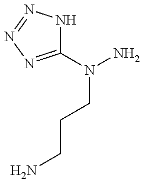 [SMILES: NCCCN(N)c1nnn[nH]1] | A |
| 22 | 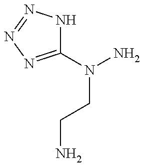 | A |
| 23 | 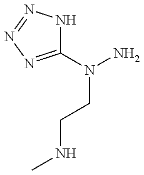 | B |
| 24 | 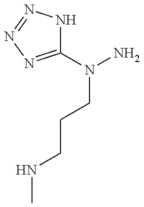 | A |
| 25 | 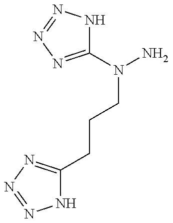 [SMILES: NN(CCCc1nnn[nH]1)c1nnn[nH]1] | B |
| 26 | 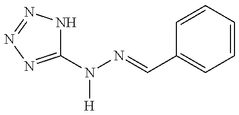 [SMILES: C(=N\Nc1nnn[nH]1)/c1ccccc1] | B |
| 27 | 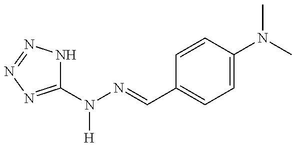 [SMILES: CN(C)c1ccc(/C=N/Nc2nnn[nH]2)cc1] | B |
| 28 | 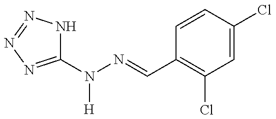 [SMILES: Clc1ccc(/C=N/Nc2nnn[nH]2)c(Cl)c1] | C |
| 29 | 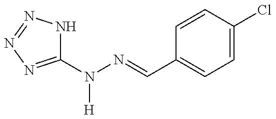 [SMILES: Clc1ccc(/C=N/Nc2nnn[nH]2)cc1] | A |
| 30 | 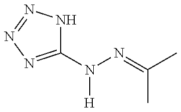 | A |
| 31 | 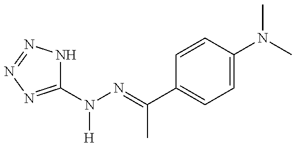 [SMILES: C/C(=N\Nc1nnn[nH]1)c1ccc(N(C)C)cc1] | A |
| 32 | 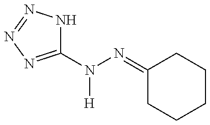 [SMILES: C1CCC(=NNc2nnn[nH]2)CC1] | A |
| 33 | 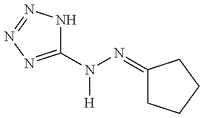 [SMILES: C1CCC(=NNc2nnn[nH]2)C1] | A |
| 34 | 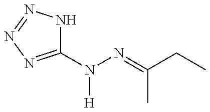 | A |
| 35 | 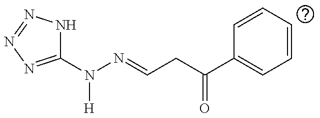 [SMILES: O=C(C/C=N/Nc1nnn[nH]1)c1ccccc1] | A |
| 36 | 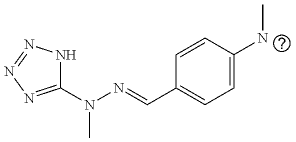 [SMILES: CNc1ccc(/C=N/N(C)c2nnn[nH]2)cc1] | B |
| 37 | 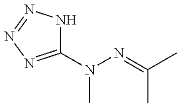 | A |
| 38 | 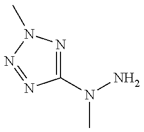 | C |
| 39 | 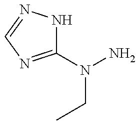 | C |
| 40 | 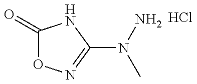 | B |
| 41 | 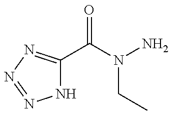 | C |
| 42 | 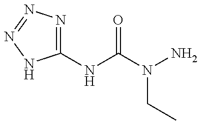 | C |
| 43 | 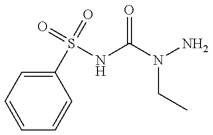 [SMILES: CCN(N)C(=O)NS(=O)(=O)c1ccccc1] | C |

IC50 (μM) A < 25 μM; 25 μM ≦ B ≦ 100 μM; C > 100 μM

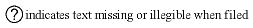

### Example 2

### Chemotherapy Induced Peripheral Neuropathy Using Paclitaxel

The method, which detects antihyperalgesic activity in rats with neuropathic pain, follows that described by Polomano et al (Pain, 94, 293-304, 2001).

Injection of the chemotherapeutic agent paclitaxel is associated with the development of a peripheral neuropathy. It is proposed that anti-hyperalgesics will reduce this chronic sign of pain hypersensitivity.

Chemotherapeutic neuropathy was produced in rats by injecting paclitaxel (2 mg/kg IP) 4 times over a 7 day period (dosing on days 1, 3, 5 &7). This results in the development of peripheral neuropathy by 3 weeks after the start of dosing. Testing was conducted on day 23 after the initiation of paclitaxel dosing.

Test compounds are dosed two hours (compound A) or five hours (compound B) prior to pain testing. Compound A was dosed 2 hours prior to testing at 30 mg/kg PO; Compound B was dosed 5 hours prior to testing at 10 and 100 mg/kg PO. Compound A is 5-(1-ethylhydrazinyl)-2H-tetrazole and Compound B is (S)-1-(2H-tetrazol-5-yl)but-3-yn-1-amine. Morphine (technical control) was dosed at 1 hour prior to testing at 5 mg/kg SC. A historical naïve data set is used to show the expected 50% withdrawal threshold (g) of animals not treated with paclitaxel. N's were 10-12 for test groups and 6 for morphine.

### Tactile Allodynia Evaluation: Manual Von Frey Test

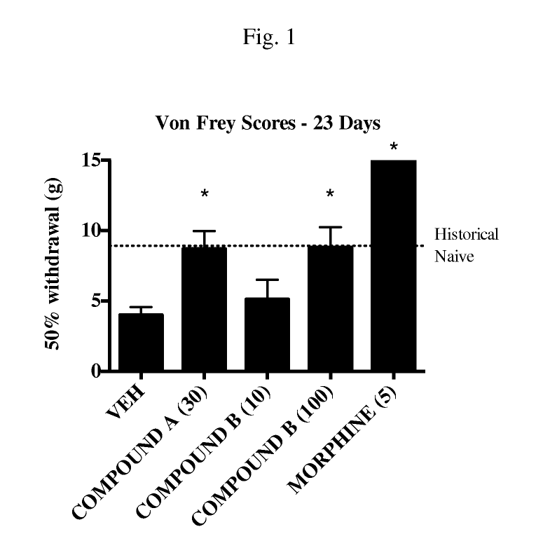

For tactile stimulation, the animal is placed under an inverted acrylic plastic box (18×11.5×14 cm) on a grid floor. The tip of a manual Von Frey probe is then applied with increasing force to the hind paw. Using the updown method published by Dixon (Journal of the American Statistical Assoc. Vol. 60, No. 312, December, 1965), filaments of progressively greater force were applied until a withdrawal of the paw was observed. At this point filaments of progressively lesser force were applied until withdrawal of the paw was absent whereupon the filament strength was increased again. This was performed on each hind paw and the scores averaged. The results can be seen in FIG. 1. Drug was compared to vehicle using ANOVA with Dunnett's post hoc MCT. *=p<0.05.

### Example 3

### Chemotherapy Induced Peripheral Neuropathy with Paclitaxel and Carboplatin

The method, which detects antihyperalgesic activity in rats with neuropathic pain, follows that described by Polomano et al (Pain, 94 293-304, 2001) with the addition of a second chemotherapeutic agent-carboplatin.

Injection of the chemotherapeutic agents paclitaxel and carboplatin is associated with the development of a peripheral neuropathy. It is proposed that anti-hyperalgesics will reduce this chronic sign of pain hypersensitivity.

Chemotherapeutic neuropathy was produced in rats by injecting paclitaxel (2 mg/kg IP) 4 times over a 7 day period (dosing on days 1, 3, 5, and 7) and carboplatin (10 mg/kg IP) 4 times over a 4 day period (dosing on days 1, 2, 3, and 4). This results in the development of peripheral neuropathy by 3 weeks after the start of dosing. Testing was conducted on day 23 after the initiation of paclitaxel dosing.

Test compounds were dosed two (Compound A) or five (Compound B) hours prior to pain testing. Compound A was tested at 3, 10, 30 mg/kg PO. Test Compound A is 5-(1-ethylhydrazinyl)-2H-tetrazole. Compound B was tested at 10, 30, 100 mg/kg PO. Test Compound B is (S)-1-(2H-tetrazol-5-yl)but-3-yn-1-amine. Morphine (technical control) was dosed at 1 hour prior to testing at 5 mg/kg IP. A naïve data set is included to show the 50% withdrawal threshold (g) of animals not treated with paclitaxel or carboplatin.

Statistical analysis is made between vehicle and Compound A/B treated groups using ANOVA with Dunnett's post hoc MCT. Typically the technical control and naïve groups are compared with vehicle treated animals separately to validate the assay. N's were 10-12 for all groups with the exception of naïves (n=6 for compound A experiment).

### Tactile Allodynia Evaluation: Electronic Von Frey Test

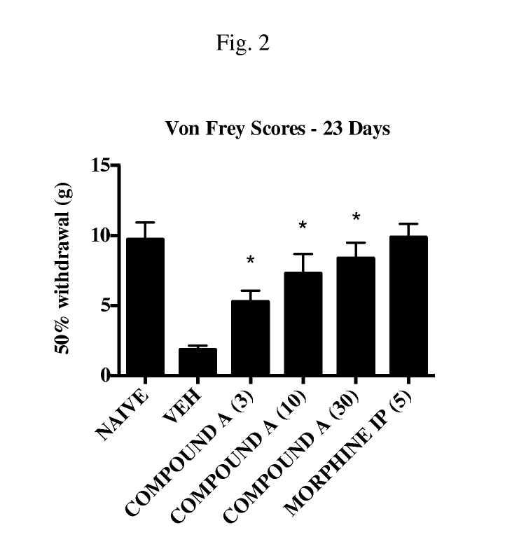

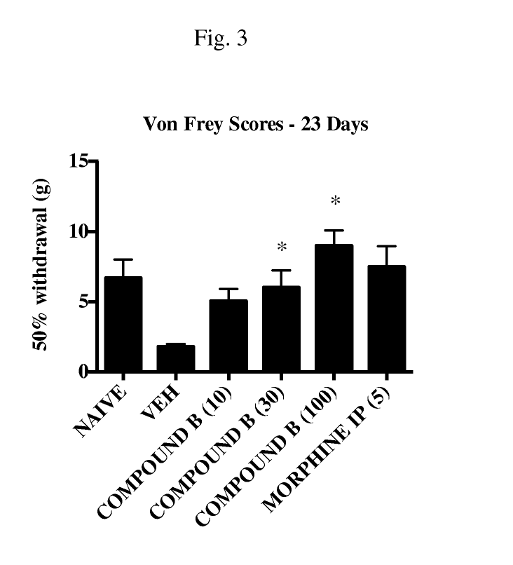

For tactile stimulation, the animal is placed under an inverted acrylic plastic box (18×11.5×14 cm) on a grid floor. The tip of a manual Von Frey probe is then applied with increasing force to the hind paw. Using the updown method published by Dixon (Journal of the American Statistical Assoc. Vol. 60, No. 312, December, 1965), filaments of progressively greater force were applied until a withdrawal of the paw was observed. At this point filaments of progressively lesser force were applied until withdrawal of the paw was absent whereupon the filament strength was increased again. This was performed on each hind paw and the scores averaged. The results can be seen in FIGS. 2 and 3. Drug was compared to vehicle using ANOVA with Dunnett's post hoc MCT. *=p<0.05.

### Example 4

### A Study to Evaluate the Effect of a Compound of Formula (I), (II), (III), (IV), (V), (VI), (VII), (VIII), (IX), or (X) in the Treatment of Patients with Chemotherapy Induced Peripheral Neuropathy (CIPN)

The purpose of this study is to evaluate the effect of a compound of Formula (I), (II), (III), (IV), (V), (VI), (VII), (VIII), (IX), or (X) on taxane induced peripheral neuropathy scores after 6 weeks treatment and is based on the separate assessment of pain and dysesthesia scores.

| Condition | Intervention | Phase |
|---|---|---|
| Chemotherapy-Induced | Drug: Compound of Formula | Phase 2 |
| Peripheral Neuropathy | (I), (II), (III), (IV), (V), (VI), | |
| | (VII), (VIII), (IX), or (X) | |
| | Drug: Placebo | |

Study Type: Interventional

### Study Design

Allocation: Randomized

Endpoint Classification: Safety/Efficacy Study

Intervention Model: Parallel Assignment

Masking: Double Blind (Subject, Investigator)

Primary Purpose: Treatment

### Primary Outcome Measures

The primary outcome will be the measure of the percentage of responders defined as patients with a minimum decrease of 50% of their maximum neuropathic pain dimension (either pain or dysesthesia) present at baseline. [Time Frame: The mean pain score during the last 7 days of the treatment period will be compared to the mean score of the last 7 days of the screening score period.] [Designated as safety issue: No]

### Secondary Outcome Measures

Assessment with respect to placebo of the effect of a compound of Formula (I), (II), (III), (IV), (V), (VI), (VII), (VIII), (IX), or (X) on Neuropathic Pain Inventory Score (total and by dimension) [Time Frame: Screening visit; V0; V1; V2; V3; V4; V5] [Designated as safety issue: No]

Assessment with respect to placebo of the effect of a compound of Formula (I), (II), (III), (IV), (V), (VI), (VII), (VIII), (IX), or (X) on Short-Form BPI [Time Frame: Screening visit; V0; V1; V2; V3; V4; V5] [Designated as safety issue: No]

Assessment with respect to placebo of the effect of a compound of Formula (I), (II), (III), (IV), (V), (VI), (VII), (VIII), (IX), or (X) on Quality of life questionnaire (CIPN 20) [Time Frame: V0; V3; V5] [Designated as safety issue: No]

Assessment with respect to placebo of the effect of a compound of Formula (I), (II), (III), (IV), (V), (VI), (VII), (VIII), (IX), or (X) on Quantitative Sensory Testing [Time Frame: V0; V3; V5] [Designated as safety issue: No]

Assessment with respect to placebo of the effect of a compound of Formula (I), (II), (III), (IV), (V), (VI), (VII), (VIII), (IX), or (X) on the use of rescue medication [Time Frame: Screening visit; V0; V1; V2; V3; V4; V5] [Designated as safety issue: No]

Assessment with respect to placebo of the effect of a compound of Formula (I), (II), (III), (IV), (V), (VI), (VII), (VIII), (IX), or (X) on safety profile [Time Frame: Screening visit; V0; V1; V2; V3; V4; V5] [Designated as safety issue: Yes]

Assessment with respect to placebo of the effect of a compound of Formula (I), (II), (III), (IV), (V), (VI), (VII), (VIII), (IX), or (X) on Global Impression of change as assessed by Investigator [Time Frame: V1; V2; V3; V4; V5] [Designated as safety issue: No]

Assessment with respect to placebo of the effect of a compound of Formula (I), (II), (III), (IV), (V), (VI), (VII), (VIII), (IX), or (X) on Global Impression of change as assessed by patient [Time Frame: V1; V2; V3; V4; V5] [Designated as safety issue: No]

Assessment with respect to placebo of the effect of a compound of Formula (I), (II), (III), (IV), (V), (VI), (VII), (VIII), (IX), or (X) on Hospital Anxiety and Depression scale [Time Frame: V0; V3; V5] [Designated as safety issue: No]

| Arms | Assigned Interventions |
|---|---|
| Experimental: 001 | Drug: Compound of Formula |
| Compound of Formula (I), (II), | (I), (II), (III), (IV), (V), (VI), |
| (III), (IV), (V), (VI), (VII), (VIII), | (VII), (VIII), (IX), or (X) |
| (IX), or (X) capsule (1, 3 or 10 | |
| milligrams) once a day | |
| Placebo Comparator: 002 | Drug: Placebo |
| Placebo capsule once a day | |

### DETAILED DESCRIPTION

The current study is a randomized (study drug assigned by chance), double-blind (neither the study doctor nor the patient knows the name of the assigned drug), placebo-controlled, dose-ranging study to evaluate the effect of a compound of Formula (I), (II), (III), (IV), (V), (VI), (VII), (VIII), (IX), or (X) in patients with Chemotherapy Induced Peripheral Neuropathy. At the start of the study, patients will be randomized to one of four groups: Compound of Formula (I), (II), (III), (IV), (V), (VI), (VII), (VIII), (IX), or (X) capsule once a day (1, 3 or 10 milligrams), or placebo once a day. Each treatment will be administered for 6 weeks. Additionally, patients will have the option to continue treatment for another 6 weeks duration.

Eligibility

Ages Eligible for Study: 18 Years to 80 Years

Genders Eligible for Study: Both

Accepts Healthy Volunteers: No

Inclusion Criteria:

Have signed an Informed Consent to participate to the trial before any study related procedure has taken place.

Have paclitaxel (or other taxane) induced peripheral neuropathy assessed by the presence of a NCI-CTC version 2 neuropathy sensory grade >/=2.

Peripheral neuropathy as clinically diagnosed during the neurological examination including sensitivity, motor function and deep tendon reflex assessments.

With Neuropathic pain as assessed by the presence of measurable pain perception (previous 24 h) on the Likert numerical rating scale >/=4 points at the screening visit and confirmed on DN4 with a score >/=4 and/or Dysesthesia as assessed by the presence of measurable dysesthesia (previous 24 h) on the Likert numerical rating scale >/=4 points at the screening visit.

Persistent neuropathy for at least 3, but no more than 12 months after the end of chemotherapy.

Be either pain treatment naive or have important side effects or inadequate relief from their current pain medication (stable over last month).

Exclusion Criteria:

Have a documented neuropathy or risk factors of neuropathy which might interfere with the assessment of the severity of pain (eg, including, but not limited to, type 2 diabetes, peripheral vascular disease, B12 Vitamin deficiency, thyroid dysfunction, post surgical neuropathic pain, post-traumatic neuropathy, or neuropathy in relation with disease progression).

Have other neurological diseases that may produce weakness, sensory loss, or autonomic symptoms, or laboratory test abnormality.

Refractory to treatment defined as not improved, according to the Investigator, by 3 or more treatments prescribed for the current PN symptoms.

HIV positive serology.

History of, or current cardiac dysrhythmias and/or a history of cardiovascular disease, including myocardial infarction, except patients with only well controlled hypertension.

Have had prior (within the past 6 months) or have concurrent neurotoxic drugs (e.g., but not limited to, cisplatin, vincristine, vinblastine, cytarabine, thalidomide, bortezomib, or procarbazine, capecitabine, navelbine).

Have a current medication that may have a similar mechanism of action as a compound of Formula (I), (II), (III), (IV), (V), (VI), (VII), (VIII), (IX), or (X): acetyl-L-carnitine

Have a current medication that could interfere with a compound of Formula (I), (II), (III), (IV), (V), (VI), (VII), (VIII), (IX), or (X) pharmacokinetics

Have current medications that could interfere with a compound of Formula (I), (II), (III), (IV), (V), (VI), (VII), (VIII), (IX), or (X) absorption.

Have a current medication of lipid lowering agents other than statins.

Have a recent history (within the previous 6 months) or current evidence of alcohol or drug abuse.

Have concurrent unstable disease involving any system (eg, advanced carcinoma other than carcinoma justifying the recent treatment with taxanes, myocardial infarction, clinical or ECG signs of myocardial ischemia, cardiac insufficiency, anginal symptoms, current symptoms of CAD, renal impairment, or any other condition that in the opinion of the Investigator would make the patient unsuitable for study participation)

Be pregnant female or lactating.

Have renal impairment defined as blood creatinine >1.5× upper limit of normal (ULN)

Hemostasis disorders or current treatment with oral anticoagulants.

Have hepatic impairment hepatic function as follows: liver enzymes (ALT and AST) >2×ULN or >3.5×ULN in case of liver metastasis.

Are not able to comply with regard to the known contraindications, warnings and precautions, drug-interactions and dosing recommendations of paracetamol or tramadol.

Be possibly dependent on the Investigator or the Sponsor (eg, including, but not limited to, affiliated employee).

Participated in any other investigational drug or therapy study with a non approved medication within the previous 3 months.

Known hypersensitivity to one of the capsules' ingredients.

Any other condition which, in the opinion of the investigator would impede competence or compliance or possibly hinder completion of the study.

### Example 5

### A Study Comparing the Effect of a Compound of Formula (I), (II), (III), (IV), (V), (VI), (VII), (VIII), (IX), or (X) to Placebo in Subjects with Neuropathic Pain Associated with HIV Neuropathy

Purpose: To evaluate the efficacy of a compound of Formula (I), (II), (III), (IV), (V), (VI), (VII), (VIII), (IX), or (X) compared to Placebo in subjects with neuropathic pain associated with HIV neuropathy.

| Condition | Intervention | Phase |
|---|---|---|
| Neuropathy | Drug: Compound of Formula (I), (II), (III), | Phase 3 |
| | (IV), (V), (VI), (VII), (VIII), (IX), or (X) | |
| | Drug: Placebo | |

Study Type: Interventional

### Study Design:

Allocation: Randomized

Endpoint Classification: Safety/Efficacy Study

Intervention Model: Parallel Assignment

Masking: Double Blind (Subject, Caregiver, Investigator)

Primary Purpose: Treatment

### Primary Outcome Measures:

Change From Baseline in Mean Pain Score at Endpoint (up to Week 16) [Time Frame: Baseline, Endpoint (up to Week 16) Designated as safety issue: No]

Mean pain score defined as the mean of the last 7 daily diary pain ratings. Participants rate their Human Immunodeficiency Virus (HIV) neuropathy pain over the past 24 hours on an 11-point numeric rating scale ranging from 0=no pain to 10=worst possible pain. A rating of 1-3 is considered as mild pain; 4-6=moderate pain; and 7-10=severe pain. Endpoint is the last observation for a participant assessed using specified imputation method, modified Baseline Observation Carried Forward (mBOCF).

### Secondary Outcome Measures:

Number of Participants With Categorical Scores on Patient Global Impression of Change (PGIC) [Time Frame: Week 16] [Designated as safety issue: No]

Number of Participants With Categorical Scores on Clinician Global Impression of Change (CGIC) [Time Frame: Week 16] [Designated as safety issue: No]

Change From Baseline in Numeric Rating Scale (NRS)-Sleep Interference Score at Week 1, 2, 3, 4, 5, 6, 7, 8, 9, 10, 11, 12, 13, 14, 15, 16 and Endpoint (up to Week 16) [Time Frame: Baseline, Week 1, 2, 3, 4, 5, 6, 7, 8, 9, 10, 11, 12, 13, 14, 15, 16, Endpoint (up to Week 16)] [Designated as safety issue: No]

Change From Baseline in Numeric Rating Scale (NRS)-Current Pain Score at Week 1, 2, 3, 4, 5, 6, 7, 8, 9, 10, 11, 12, 13, 14, 15, 16 and Endpoint (up to Week 16) [Time Frame: Baseline, Week 1, 2, 3, 4, 5, 6, 7, 8, 9, 10, 11, 12, 13, 14, 15, 16, Endpoint (up to Week 16)] [Designated as safety issue: No]

Change From Baseline in Brief Pain Inventory-Short Form (BPI-sf) Score at Week 4, 8, 12, 16 and Endpoint (up to Week 16) [Time Frame: Baseline, Week 4, 8, 12, 16, Endpoint (up to Week 16)] [Designated as safety issue: No]

Change From Baseline in Neuropathic Pain Symptom Inventory (NPSI) Item Scores at Endpoint (up to Week 16) [Time Frame: Baseline, Endpoint (up to Week 16)] [Designated as safety issue: No]

Neuropathic Pain Symptom Inventory (NPSI): Change From Baseline in Number of Participants With Duration of Spontaneous Pain and Number of Pain Attacks at Endpoint (up to Week 16) [Time Frame: Baseline, Endpoint (up to Week 16)] [Designated as safety issue: No]

Change From Baseline in Neuropathic Pain Symptom Inventory (NPSI) Subscales and Total Intensity Score at Endpoint (up to Week 16) [Time Frame: Baseline, Endpoint (up to Week 16)] [Designated as safety issue: No]

Total Sleep Time (TST) and Minutes of Interrupted Sleep (MIS) [Time Frame: Baseline (Day −14 to 1), Week 1 through Week 4, Week 12 through Week 16, Endpoint (up to Week 16)] [Designated as safety issue: No]

Sleep Fragmentation Index (SFI) [Time Frame: Baseline (Day −14 to 1), Week 1 through Week 4, Week 12 through Week 16, Endpoint (up to Week 16)] [Designated as safety issue: No]

Sleep Efficiency [Time Frame: Baseline (Day −14 to 1), Week 1 through Week 4, Week 12 through Week 16, Endpoint (up to Week 16)] [Designated as safety issue: No]

Total Activity Counts [Time Frame: Baseline (Day −14 to 1), Week 1 through Week 4, Week 12 through Week 16, Endpoint (up to Week 16)] [Designated as safety issue: No]

Percentage Day Time Above Sedentary Level [Time Frame: Baseline (Day −14 to 1), Week 1 through Week 4, Week 12 through Week 16, Endpoint (up to Week 16)] [Designated as safety issue: No]

Change From Baseline in Medical Outcomes Study-Sleep Scale (MOS-SS) at Endpoint (up to Week 16) [Time Frame: Baseline, Endpoint (up to Week 16)] [Designated as safety issue: No]

Medical Outcomes Study-Sleep Scale (MOS-SS): Number of Participants With Optimal Sleep [Time Frame: Baseline, Endpoint (up to Week 16)] [Designated as safety issue: No]

Change From Baseline in Hospital Anxiety and Depression Scales (HADS) at Endpoint (up to Week 16) [Time Frame: Baseline, Endpoint (up to Week 16)] [Designated as safety issue: No]

| Arms | Assigned Interventions |
|---|---|
| Experimental: Compound of Formula | Drug: Compound of Formula |
| (I), (II), (III), (IV), (V), (VI), (VII), | (I), (II), (III), (IV), (V), (VI), |
| (VIII), (IX), or (X) | (VII), (VIII), (IX), or (X) |
| | 1-30 mg, once daily |
| Placebo Comparator: Placebo | Drug: Placebo comparator |
| | Study duration is approximately |
| | 19 weeks |

Eligibility

Ages Eligible for Study: 18 Years and older

Genders Eligible for Study: Both

Accepts Healthy Volunteers: No

Inclusion Criteria

Men and women, ages of 18 or greater.

Documented evidence of HIV-1 infection.

Documented diagnosis of HIV-associated Distal Symmetrical Polyneuropathy (DSP) with subjective sensory symptom of pain.

Pain starts in the feet.

Exclusion Criteria

Subject has untreated vitamin B12 deficiency (serum B12 level <200 pg/ml) or if treated B12 deficiency-treatment is less than 6 months of B12 supplementation (injection or intranasal B12) prior to screening.

Diabetes mellitus requiring regular medical treatment (other than diet and exercise) or HbA1C >6.9.

Subjects with peripheral neuropathic pain that is not associated with HIV infection; including subjects with conditions such as: Post Herpetic Neuralgia (PHN), Diabetic Peripheral Neuropathy (DPN), familial neuropathies; compression related neuropathy, radicular pain, other infection related neuropathies (eg, leprosy); neuropathy related to: metabolic abnormalities; nutritional factors; vascular insults; inflammation; autoimmune disease; and malignancy.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for treating chemotherapy-induced peripheral neuropathy, or human immunodeficiency virus-associated sensory neuropathy, or symptoms thereof, comprising administering to an individual in need thereof a therapeutically effective amount of a compound of Formula (I) having the structure: 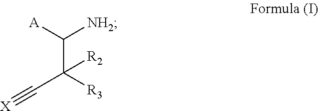
wherein:
A is a carboxylic acid isostere;
X is CR₁, or N;
R₁ is H, substituted or unsubstituted alkyl, substituted or unsubstituted heteroalkyl, substituted or unsubstituted heterocycloalkyl, substituted or unsubstituted aryl, or substituted or unsubstituted heteroaryl; and
R₂ and R₃ are each independently H, substituted or unsubstituted alkyl, or substituted or unsubstituted heteroalkyl; or R₂ and R₃ together with the carbon to which they are attached form a cycloalkyl or heterocycloalkyl ring;
or a pharmaceutically acceptable salt, solvate, or prodrug thereof.

2. The method of claim 1 having the structure of Formula (II):

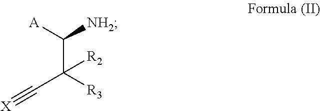

3. The method of claim 2 wherein A is a carboxylic acid isostere selected from

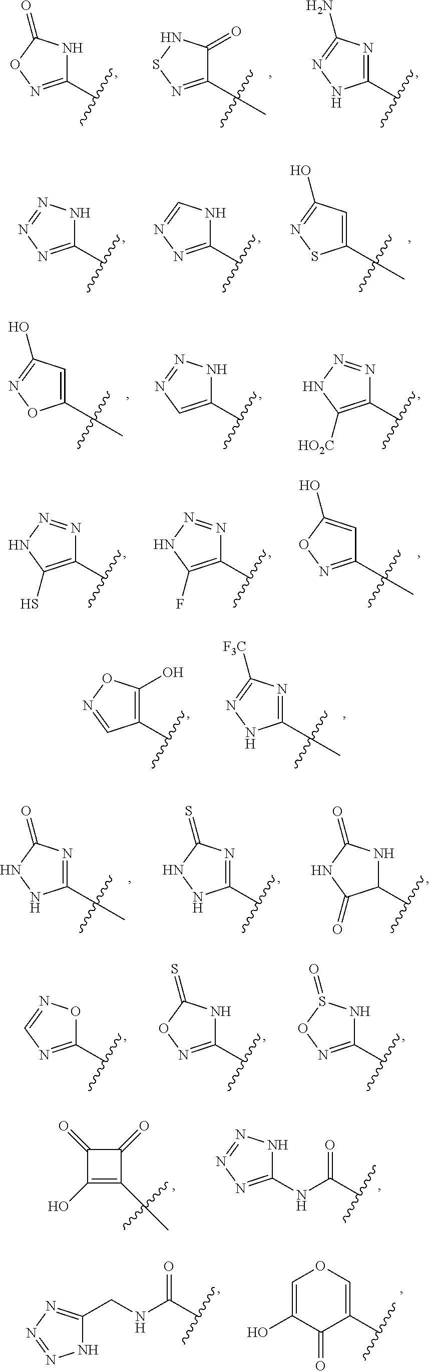

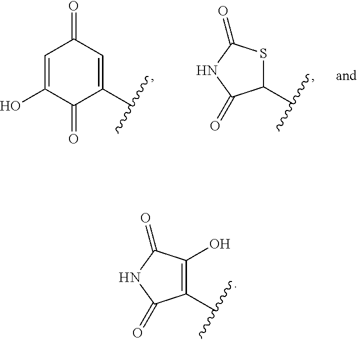 [SMILES: *C1=CC(=O)C=C(O)C1=O ; *C1SC(=O)NC1=O ; *C1=C(O)C(=O)NC1=O]

4. The method of claim 2 wherein A is a carboxylic acid isostere selected from —SO₃H, —SO₂NHR₄, —P(O)(OR₄)₂, —P(O)(R₄)(OR₄), —CON(R₄)₂, —CONHNHSO₂R₄, —CONHSO₂R₄, —B(OR₅)₂, —C(R₄)₂B(OR₅)₂, and —CON(R₄)C(R₄)₂B(OR₅)₂; wherein each R₄ is independently H, OH, substituted or unsubstituted alkyl, or substituted or unsubstituted aryl; and R₅ is H or C₁-C₆alkyl.

5. The method of claim 3 wherein X is N.

6. The method of claim 3 wherein X is CR₁, wherein R₁ is H, substituted or unsubstituted alkyl, or substituted or unsubstituted heteroalkyl.

7. (canceled)

8. The method of claim 6 wherein R₁ is H.

9. (canceled)

10. (canceled)

11. (canceled)

12. (canceled)

13. (canceled)

14. The method of claim 8 wherein A is

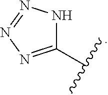 [SMILES: *c1nnn[nH]1]

15. The method of claim 8 wherein A is

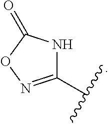 [SMILES: *c1noc(=O)[nH]1]

16. The method of claim 8 wherein A is

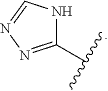 [SMILES: *c1nnc[nH]1]

17. The method of claim 8 wherein R₂ and R₃ are each H.

18. The method of claim 2 having the structure:

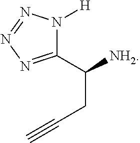

19. The method of claim 2 having the structure:

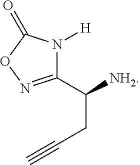

20. The method of claim 2 having the structure:

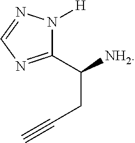

21. (canceled)

22. (canceled)

23. (canceled)

24. (canceled)

25. (canceled)

26. (canceled)

27. (canceled)

28. (canceled)

29. (canceled)

30. A method for treating chemotherapy-induced peripheral neuropathy or human immunodeficiency virus-associated sensory neuropathy, or symptoms thereof, comprising administering to an individual in need thereof a therapeutically effective amount of a compound of Formula (VIII) having the structure: 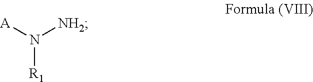
wherein:
A is 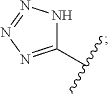 [SMILES: *c1nnn[nH]1]
and
R₁ is substituted or unsubstituted C₂-C₆alkyl, substituted or unsubstituted heteroalkyl, substituted or unsubstituted heterocycloalkyl, substituted or unsubstituted aryl, or substituted or unsubstituted heteroaryl; or a pharmaceutically acceptable salt, solvate, or prodrug thereof.

31. The method of claim 30 wherein R₁ is substituted or unsubstituted C₂-C₆alkyl, or substituted or unsubstituted heteroalkyl.

32. The method of claim 31 wherein R₁ is substituted or unsubstituted heteroalkyl.

33. The method of claim 31 wherein R₁ substituted or unsubstituted C₂-C₆alkyl.

34. The method of claim 33 wherein R₁ is —CH₂CH₃.

35. (canceled)

36. (canceled)

37. (canceled)

38. (canceled)

39. The method of claim 1 or claim 30 for treating chemotherapy-induced peripheral neuropathy or symptoms thereof.

40. The method of claim 39 wherein the chemotherapy-induced peripheral neuropathy is associated with one or more chemotherapeutic agents selected from docetaxel, paclitaxel, cisplatin, carboplatin, oxaliplatin, vincristine, thalidomide, suramin, bortezomib, rituximab, cyclophosphamide, doxorubicin, abraxane, cabazitaxel, vinorelbine, vinblastine, etoposide, ixabepilone, lenalidomide, pomalidomide, carfilzomib, eribulin, 5-fluorouracil, leucovorin, and gemcitabine.

41. The method of claim 39 wherein the chemotherapy-induced peripheral neuropathy is associated with two chemotherapeutic agents selected from paclitaxel and carboplatin.

42. The method of claim 39 wherein the chemotherapy-induced peripheral neuropathy is associated with one or two chemotherapeutic agents selected from a taxane and a platinum based therapeutic.

43. (canceled)

44. The method of claim 1 or claim 30, further comprising administration of a second agent selected from cholinesterase inhibitors, adenosine inhibitors, progestational agents, opioid antagonists, partial opioid agonists, angiotensin receptor blockers, central nervous system stimulants, selective serotonin reuptake inhibitors (SSRIs), dual 5-HT-NE reuptake inhibitors (SNRI's), antidepressants, antihypertensives, calcium channel antagonists, ACE inhibitors, alpha-2 adrenergic agonists, gamma aminobutyric acid agonists, antiepileptic drugs, NSAIDs, steroids, and glutamate antagonists.

45. The method of claim 1 or claim 30, further comprising administration of a second agent selected from acetazolamide, theophylline, progesterone, donepezil, naloxone, nicotine, paroxetine, protriptyline, metoprolol, cilazapril, propranolol, atenolol, hydrochlorothiazide, isradipine, spirapril, doxapram, clonidine, baclofen, sabeluzole, gabapentin, pregablin, duloxetine, morphine, codeine, tramadol, dextropropoxyphene, dihydrocodeine, hydrocodone, oxycodone, losartan, irbesartan, olmesartan, candesartan, telmisartan, azilsartan, naproxen, ketoprofen, acetaminophen, and diclofenac.
